(19) United States
(12) Patent Application Publication
(10) Pub. No.: US 2007/0021414 A1
(43) Pub. Date: Jan. 25, 2007

(54) 1-'2-(4-Hydroxyphenyl)-2-hydroxyethyl!-piperidin-4-ol compounds as nmda receptor antagonists

(75) Inventors: Kazuo Ando, Aichi-ken (JP); Masako Hirota, Aichi-ken (JP)

Correspondence Address: WARNER-LAMBERT COMPANY, 2800 PLYMOUTH RD, ANN ARBOR, MI, 48105, US

(73) Assignee: PFIZER, INC.

(21) Appl. No.: 10/575,383

(22) PCT Filed: Sep. 27, 2004
(86) PCT No.: PCT/IB04/03127

§ 371 Date: Apr. 7, 2006

Related U.S. Application Data
(60) Provisional application No. 60/509,643, filed on Oct. 8, 2003.

(51) Int. Cl. A61K 31/541; A61K 31/5377; A61K 31/496; A61K 31/454; A61K 31/445; C07D 403/02; C07D 401/02; C07D 413/02; C07D 417/02

(52) U.S. Cl. 514/227.5; 514/235.2; 514/253.1; 514/317; 514/326; 544/60; 544/128; 544/360; 546/207; 546/216

(57) ABSTRACT

This invention provides a compound of the formula (I), wherein R¹ and R² independently represents a hydrogen atom or the like; R³ represents an aryl group having from 6 to 10 ring carbon or the like; said aryl groups having from 6 to 10 ring carbon atoms and said heteroaryl groups having from 5 to 10 atoms are unsubstituted or are substituted by at least one substituent selected from the group consisting of substituents a; said substituents a are selected from the group consisting of halogen atoms or the like; or a pharmaceutically acceptable ester of such compound, or a pharmaceutically acceptable salt thereof. These compounds are useful for the treatment of disease conditions caused by overactivation of NMDA NR2B receptor such of pain, or the like in mammalian. This invention also provides a pharmaceutical composition comprising the above compound.

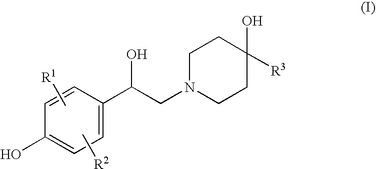

### TECHNICAL FIELD

This invention relates to novel 3,4-dihydroquinolin-2(1H)-one compounds.

These compounds are useful as antagonists of NMDA (N-methyl-D-aspartate) NR2B receptor, and are thus useful for the treatment of pain, stroke, traumatic brain injury, Parkinson's disease, Alzheimer's disease, depression, anxiety, migraine, or the like in mammalian, especially humans. The present invention also relates to a pharmaceutical composition comprising the above compounds.

### BACKGROUND ART

Glutamate plays dual role in the central nervous system (CNS) as essential amino acid and the principal excitatory neurotrarsmitters. There are at least four classes of receptors, specifically N-methyl-aspartate (NMDA), 2-amino-3-(methyl-3-hydroxyisoxazol-4-yl)propionic acid (AMPA), kainate and metabotropic. There is considerable preclinical evidence that hyperalgesia and allodynia following peripheral tissue or nerve injury is not only due to an increase in the sensitivity of primary afferent nociceptors at the site of injury but also depends on NMDA receptor-mediated central changes in synaptic excitability. In humans, NMDA receptor antagonists have also been found to decrease both pain perception and sensitization. Also, overactivation of NMDA receptor is a key event for triggering neuronal cell death under pathological conditions of acute and chronic forms of neurodegeneration. However, while NMDA receptor inhibition has therapeutic utility in the treatment of pain and neurodegenerative diseases, there are significant liabilities to many available NMDA receptor antagonists that can cause potentially serious side effects. NMDA subunits are differentially distributed in the CNS.

Especially, NR2B is believed to be restricted to the forebrain and laminas I and II of the dosal horn. The more discrete distribution of NR2B subunit in the CNS may support a reduced side-effect profile of agents that act selectively at this site. For example, NMDA NR2B selective antagonists may have clinical utility for the treatment of neuropathic and other pain conditions in human with a reduced side-effect profile than existing NMDA antagonists (S. Boyce, et al., Neuropharmacology, 38, pp.611-623 (1999)).

International Publication Number WO 96/06081 discloses a variety of phenol compounds. Especially, a compound represented by the following formula is disclosed in it:

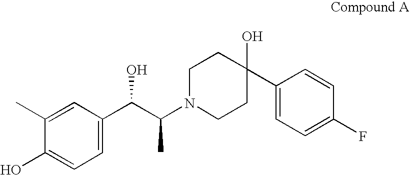

However, the known compounds have potential to prolong the QT-interval due to their potent inhibitory activity at HERG (human ether-a-go-go related gene) potassium channel. QT prolongation is known to have a potential liability to produce fatal cardiac arrhythmias of Torsades de Pointes (TdP). The ability to prolong the cardiac action potential duration was identified as being due to an action at the HERG potassium channel. For example, drugs withdrawn from the market due to QT prolongation, such as Cisapride and Terfenadine, are known to be potent HERG potassium channel blocker (Expert Opinion of Pharmacotherapy; 2, pp947-973, 2000). Therefore, it would be desirable if there were provided a novel NMDA NR2B selective antagonist with analgesic activity by systemic administration and with reduced inhibitory activity at HERG potassium channel.

### BRIEF DISCLOSURE OF THE INVENTION

It has now surprisingly been found that phenol compounds of present invention are NMDA NR2B selective antagonists with analgesic activity by systemic administration and with reduced inhibitory activity at HERG channel. Inhibitory activity at HERG channel was estimated from affinity for HERG type potassium channel was investigated by checking [³H]dofetilide binding, which can predict inhibitory activity at HERG channel (Eur. J. Pharmacol., 430, pp147-148, 2001). Selected compounds with low [³]dofetilide binding activity were evaluated in I_{HERG} assay to check activity at HERG channel. The compounds of the present invention show a reduced QT prolongation by removing a methyl group from the carbon atom adjacent to nitrogen atom on piperidine ring of the formula (I).

The present invention provides a compound of the following formula (I): 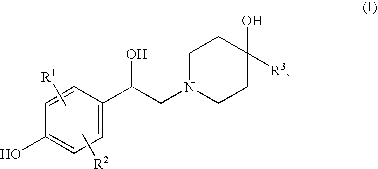
wherein R¹ and R² independently represents a hydrogen atom, a halogen atom or an alkyl group having from 1 to 6 carbon atoms;
R³ represents an aryl group having from 6 to 10 ring carbon atoms or a heteroaryl group having from 5 to 10 ring atoms which consists of from 1 to 4 heteroatoms independently selected from the group consisting of sulfur atoms, oxygen atoms and nitrogen atoms;
said aryl groups having from 6 to 10 ring carbon atoms and said heteroaryl groups having from 5 to 10 atoms are unsubstituted or are substituted by at least one substituent selected from the group consisting of substituents α;
said substituents α are selected from the group consisting of halogen atoms, alkyl groups having from 1 to 6 carbon atoms, alkoxy groups having from 1 to 6 carbon atoms or alkoxyalkyl groups having from 1 to 6 carbon atoms;
or a pharmaceutically acceptable ester of such compound,
or a pharmaceutically acceptable salt thereof.

The phenol compounds of this invention have an antagonistic action towards NMDA NR2B receptor subtype selectively and are thus useful in therapeutics, particularly for the treatment of stroke or brain injury, chronic neurodegenerative disease such as Parkinson's disease, Alzheimer's disease, Huntington's disease or amyotrophic lateral sclerosis (ALS), epilepsy, convulsive disorder, pain, anxiety, human immunodeficiency virus (HIV) related neuronal injury, migraine, depression, schizophrenia, tumor, post-anesthesia cognitive decline (PACD), glaucoma, tinnitus, tradive dyskinesia, allergic encephalomyelitis, opioid tolerance, drug abuse, alcohol abuse, Iritable bowel syndrome (IBS), or the like in mammalian, especially humans.

The compounds of the present invention are useful for the general treatment of pain, particularly neuropathic pain. Physiological pain is an important protective mechanism designed to warn of danger from potentially injurious stimuli from the external environment. The system operates through a specific set of primary sensory neurons and is exclusively activated by noxious stimuli via peripheral transducing mechanisms (Millan 1999 Prog. Neurobio. 57: 1-164 for an integrative Review). These sensory fibres are known as nociceptors and are characterized by small diameter axons with slow conduction velocities. Nociceptors encode the intensity, duration and quality of noxious stimulus and by virtue of their topographically organized projection to the spinal cord, the location of the stimulus. The nociceptors are found on nociceptive nerve fibres of which there are two main types, A-delta fibres (myelinated) and C fibres (non-myelinated). The activity generated by nociceptor input is transferred after complex processing in the dorsal horn, either directly or via brain stem relay nuclei to the ventrobasal thalamus and then on to the cortex, where the sensation of pain is generated.

Intense acute pain and chronic pain may involve the same pathways driven by pathophysiological processes and as such cease to provide a protective mechanism and instead contribute to debilitating symptoms associated with a wide range of disease states. Pain is a feature of many trauma and disease states. When a substantial injury, via disease or trauma, to body tissue occurs the characteristics of nociceptor activation are altered. There is sensitisation in the periphery, locally around the injury and centrally where the nociceptors terminate. This leads to hypersensitivity at the site of damage and in nearby normal tissue. In acute pain these mechanisms can be useful and allow for the repair processes to take place and the hypersensitivity returns to normal once the injury has healed. However, in many chronic pain states, the hypersensitivity far outlasts the healing process and is normally due to nervous system injury. This injury often leads to maladaptation of the afferent fibres (Woolf & Salter 2000 Science 288: 1765-1768). Clinical pain is present when discomfort and abnormal sensitivity feature among the patient's symptoms. Patients tend to be quite heterogeneous and may present with various pain symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Although patients with back pain, arthritis pain, CNS trauma, or neuropathic pain may have similar symptoms, the underlying mechanisms are different and, therefore, may require different treatment strategies. Therefore pain can be divided into a number of different areas because of differing pathophysiology, these include nociceptive, inflammatory, neuropathic pain etc. It should be noted that some types of pain have multiple aetiologies and thus can be classified in more than one area, e.g. Back pain, Cancer pain have both nociceptive and neuropathic components.

Nociceptive pain is induced by tissue injury or by intense stimuli with the potential to cause injury. Pain afferents are activated by transduction of stimuli by nociceptors at the site of injury and sensitise the spinal cord at the level of their termination. This is then relayed up the spinal tracts to the brain where pain is perceived (Meyer et al., 1994 Textbook of Pain 13-44). The activation of nociceptors activates two types of afferent nerve fibres. Myelinated A-delta fibres transmitted rapidly and are responsible for the sharp and stabbing pain sensations, whilst unmyelinated C fibres transmit at a slower rate and convey the dull or aching pain. Moderate to severe acute nociceptive pain is a prominent feature of, but is not limited to pain from strains/sprains, post-operative pain (pain following any type of surgical procedure), posttraumatic pain, burns, myocardial infarction, acute pancreatitis, and renal colic. Also cancer related acute pain syndromes commonly due to therapeutic interactions such as chemotherapy toxicity, immunotherapy, hormonal therapy and radiotherapy. Moderate to severe acute nociceptive pain is a prominent feature of, but is not limited to, cancer pain which may be tumour related pain, (e.g. bone pain, headache and facial pain, viscera pain) or associated with cancer therapy (e.g. postchemotherapy syndromes, chronic postsurgical pain syndromes, post radiation syndromes), back pain which may be due to herniated or ruptured intervertabral discs or abnormalities of the lumber facet joints, sacroiliac joints, paraspinal muscles or the posterior longitudinal ligament.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The inflammatory process is a complex series of biochemical and cellular events activated in response to tissue injury or the presence of foreign substances, which result in swelling and pain (Levine and Taiwo 1994: Textbook of Pain 45-56). Arthritic pain makes up the majority of the inflammatory pain population. Rheumatoid disease is one of the commonest chronic inflammatory conditions in developed countries and rheumatoid arthritis is a common cause of disability. The exact aetiology of RA is unknown, but current hypotheses suggest that both genetic and microbiological factors may be important (Grennan & Jayson 1994 Textbook of Pain 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder 2002 Ann Pharmacother. 36: 679-686; McCarthy et al., 1994 Textbook of Pain 387-395). Most patients with OA seek medical attention because of pain. Arthritis has a significant impact on psychosocial and physical function and is known to be the leading cause of disability in later life. Other types of inflammatory pain include but are not limited to inflammatory bowel diseases (IBD),

Other types of pain include but are not limited to;
Musculo-skeletal disorders including but not limited to myalgia, fibromyalgia, spondylitis, sero-negative (non-rheumatoid) arthropathies, non-articular rheumatism, dystrophinopathy, Glycogenolysis, polymyositis, pyomyositis.
Central pain or ‘thalamic pain’ as defined by pain caused by lesion or dysfunction of the nervous system including but not limited to central post-stroke pain, multiple sclerosis, spinal cord injury, Parkinson's disease and epilepsy.
Heart and vascular pain including but not limited to angina, myocardical infarction, mitral stenosis, pericarditis, Raynaud's phenomenon, scleredoma, scleredoma, skeletal muscle ischemia.
Visceral pain, and gastrointestinal disorders. The viscera encompasses the organs of the abdominal cavity. These organs include the sex organs, spleen and part of the digestive system. Pain associated with the viscera can be divided into digestive visceral pain and non-digestive visceral pain. Commonly encountered gastrointestinal (GI) disorders include the functional bowel disorders (FBD) and the inflammatory bowel diseases (BD). These GI disorders include a wide range of disease states that are currently only moderately controlled, including—for FBD, gastro-esophageal reflux, dyspepsia, the irritable bowel syndrome (IBS) and functional abdominal pain syndrome (FAPS), and—for IBD, Crohn's disease, ileitis, and ulcerative colitis, which all regularly produce visceral pain. Other types of visceral pain include the pain associated with dysmenorrhea, pelvic pain, cystitis and pancreatitis.
Head pain including but not limited to migraine, migraine with aura, migraine without aura cluster headache, tension-type headache.
Orofacial pain including but not limited to dental pain, temporomandibular myofascial pain.

The present invention provides a pharmaceutical composition for the treatment of disease conditions caused by overactivation of NMDA NR2B receptor, in a mammalian subject, which comprises administering to said subject a therapeutically effective amount of a compound of formula (I).

Further, the present invention also provides a composition which comprises a therapeutically effective amount of the cycloalkylene amide compound of formula (I) or its pharmaceutically acceptable salt together with a pharmaceutically acceptable carrier. Among them, the composition is preferably for the treatment of disease defined above.

Also, the present invention provides for the use of a compound of formula (I),or a pharmaceutically acceptable ester of such compound, or a pharmaceutically acceptable salt thereof, as a medicament.

Also, the present invention provides a method for the treatment of disease conditions defined above, which comprises administering to said subject a therapeutically effective amount of a compound of formula (I).

Further, the present invention provides a method for the treatment of disease conditions defined above in a mammal, preferably human, which comprises administering to said subject a therapeutically effective amount of a compound of formula (I).

Yet further, the present invention provides the use of a therapeutically effective amount of a compound of formula (I) in the manufacture of a medicament for the treatment of the disease conditions defined above.

### DETAILED DESCRIPTION OF THE INVENTION

As used herein, the term “halogen” means fluoro, chloro, bromo and iodo, preferably fluoro or chloro.

As used herein, the term “alkyl” means straight or branched chain saturated radicals, including, but not limited to methyl, ethyl, n-propyl, isopropyl, n-butyl, iso-butyl, secondary-butyl, tertiary-butyl.

As used herein, the term “alkoxy” means alkyl-O—, including, but not limited to methoxy, ethoxy, n-propoxy, isopropoxy, n-butoxy, iso-butoxy, secondary-butoxy, tertiary-butoxy.

As used herein, the term “alkoxyalkyl” means alkyl-O-alkyl, including, but not limited to methoxymethyl, methoxyethyl, methoxypropyl, methoxbutyl, ethoxymethyl, ethoxyethyl, ethoxypropyl, ethoxbutyl, n-propoxymethyl, n-propoxyethyl, n-propoxypropyl, n-propoxybutyl, isopropoxymethyl, isopropoxyethyl, isopropoxypropyl, isopropoxybutyl, n-butoxymethyl, n-butoxyethyl, n-butoxypropyl, iso-butoxyrnethyl, iso-butoxyethyl, iso-butoxypropyl, iso-butoxybutyl, secondary-butoxymethyl, secondary-butoxyethyl, secondary-butoxypropyl, secondary-butoxybutyl, tertiary-butoxymethyl, tertiary-butoxyethyl, tertiary-butoxypropyl or tertiary-butoxybutyl.

As used herein, the term “aryl” means a monocyclic or bicyclic aromatic carbocyclic ring of 6 to 10 carbon atoms, including, but not limited to, phenyl or naphtyl, preferably phenyl.

The term “heteroaryl” means a 5- to 10-membered monocyclic or bicyclic aromatic heterocyclic ring which consists of from 1 to 4 heteroatoms independently selected from the group consisting of suilfr atoms, oxygen atoms and nitrogen atoms including, but not limited to, pyrazolyl, furyl, thienyl, oxazolyl, tetrazolyl, thiazolyl, imidazolyl, thiadiazolyl, pyridyl, pyrimidinyl, pyrrolyl, thiophenyl, pyrazinyl, pyridazinyl, isooxazolyl, isothiazolyl, triazolyl, flrazanyl, quinolyl, isoquinolyl, tetrahydroquinolyl, tetrahydroisoquinolyl, chromanyl or isochromanyl group, and the like.

The term “ordinary protecting group” means a protecting group, which can be cleaved by a chemical method such as hydrogenolysis, hydrolysis, electrolysis or photolysis.

The term “esters” means a protecting group which can be cleaved in vivo by a biological method such as hydrolysis and forms a free acid or salt thereof. Whether a compound is such a derivative or not can be determined by administering it by intravenous injection to an experimental animal, such as a rat or mouse, and then studying the body fluids of the animal to determine whether or not the compound or a pharmaceutically acceptable salt thereof can be detected.

Preferred examples of groups for an ester of a hydroxy group include: lower aliphatic alkanoyl groups, for example: alkanoyl groups, such as the formyl, acetyl, propionyl, butyryl, isobutyryl, pentanoyl, pivaloyl, valeryl, isovaleryl, octanoyl, nonanoyl, decanoyl, 3-methylnonanoyl, 8-methylnonanoyl, 3-ethyloctanoyl, 3,7-dimethyloctanoyl, undecanoyl, dodecanoyl, tridecanoyl, tetradecanoyl, pentadecanoyl, hexadecanoyl, 1-methylpentadecanoyl, 14-methylpentadecanoyl, 13,13-dimethyltetradecanoyl, heptadecanoyl, 15-methylhexadecanoyl, octadecanoyl, 1-methylheptadecanoyl, nonadecanoyl, icosanoyl and henicosanoyl groups; halogenated alkylcarbonyl groups, such as the chloroacetyl, dichloroacetyl, trichloroacetyl, and trifluoroacetyl groups; alkoxyalkylcarbonyl groups, such as the methoxyacetyl group; and unsaturated alkylcarbonyl groups, such as the acryloyl, propioloyl, methacryloyl, crotonoyl, isocrotonoyl and (1)-2-methyl-2-butenoyl groups; more preferably, the lower aliphatic alkanoyl groups having from 1 to 6 carbon atoms; aromatic alkanoyl groups, for example: arylcarbonyl groups, such as the benzoyl, α-naphthoyl and β-naphthoyl groups; halogenated arylcarbonyl groups, such as the 2-bromobenzoyl and 4-chlorobenzoyol groups; lower alkylated arylcarbonyl groups, such as the 2,4,6-trimethylbenzoyl and 4-toluoyl groups; lower alkoxylated arylcarbonyl groups, such as the 4-anisoyl group; nitrated arylcarbonyl groups, such as the 4-nitrobenzoyl and 2-nitrobenzoyl groups; lower alkoxycarbonylated arylcarbonyl groups, such as the 2-(methoxycarbonyl)benzoyl group; and arylated arylcarbonyl groups, such as the 4-phenylbenzoyl group; alkoxycarbonyl groups, for example: lower alkoxycarbonyl groups, such as the methoxycarbonyl, ethoxycarbonyl, propoxycarbonyl, butoxycarbonyl, sec-butoxycarbonyl, t-butoxycarbonyl and isobutoxycarbonyl groups; and halogen- or tri(lower alkyl)silyl-substituted lower alkoxycarbonyl groups, such as the 2,2,2-trichloroethoxycarbonyl and 2-trimethylsilylethoxycarbonyl groups; tetrahydropyranyl or tetrahydrothiopyranyl groups, such as: tetrahydropyran-2-yl, 3-bromotetrahydropyran-2-yl, 4-methoxytetrahydropyran-4-yl, tetrahydrothiopyran-2-yl, and 4-methoxytetrahydrothiopyranyl groups; tetrahydrofuranyl or tetrahydrothiofuranyl groups, such as: tetrahydrofuran-2-yl and tetrahydrothiofuran-2-yl groups; silyl groups, for example: tri(lower alkyl)silyl groups, such as the trimethylsilyl, triethylsilyl, isopropyldimethylsilyl, t-butyldimethylsilyl, methyldiisopropylsilyl, methyldi-t-butylsilyl and triisopropylsilyl groups; and tri(lower alkyl)silyl groups substituted by 1 or 2 aryl groups, such as the diphenylmethylsilyl, diphenylbutylsilyl, diphenylisopropylsilyl and phenyldiisopropylsilyl groups; alkoxymethyl groups, for example: lower alkoxymethyl groups, such as the methoxymethyl, 1,1-dimethyl-1-methoxymethyl, ethoxymethyl, propoxymethyl, isopropoxymethyl, butoxymethyl and t-butoxymethyl groups; lower alkoxylated lower alkoxymethyl groups, such as the 2-methoxyethoxymethyl group; and halo(lower alkoxy)methyl groups, such as the 2,2,2-trichloroethoxymethyl and bis(2-chloroethoxy)methyl groups; substituted ethyl groups, for example: lower alkoxylated ethyl groups, such as the 1-ethoxyethyl and 1-(isopropoxy)ethyl groups; and halogenated ethyl groups, such as the 2,2,2-trichloroethyl group; aralkyl groups, for example: lower alkyl groups substituted by from 1 to 3 aryl groups, such as the benzyl, α-naphthylmethyl, β-naphthylmethyl, diphenylmethyl, triphenylmethyl, α-naphthyldiphenylmethyl and 9-anthrylmethyl groups; and lower alkyl groups substituted by from 1 to 3 substituted aryl groups, where one or more of the aryl groups is substituted by one or more lower alkyl, lower alkoxy, nitro, halogen or cyano substituents, such as the 4-methylbenzyl, 2,4,6-trimethylbenzyl, 3,4,5-trimethylbenzyl, 4-methoxybenzyl, 4-methoxyphenyldiphenylmethyl, 2-nitrobenzyl, 4-nitrobenzyl, 4-chlorobenzyl, 4-bromobenzyl and 4-cyanobenzyl groups; alkenyloxycarbonyl groups: such as the vinyloxycarbonyl and aryloxycarbonyl groups; and aralkyloxycarbonyl groups in which the aryl ring may be substituted by 1 or 2 lower alkoxy or nitro groups: such as the benzyloxycarbonyl, 4-methoxybenzyloxycarbonyl, 3,4-dimethoxybenzyloxycarbonyl, 2-nitrobenzyloxycarbonyl and 4-nitrobenzyloxycarbonyl groups.

The term “treating”, as used herein, refers to reversing, alleviating, inhibiting the progress of, or preventing the disorder or condition to which such term applies, or one or more symptoms of such disorder or condition. The term “treatment” as used herein refers to the act of treating, as “treating” is defined immediately above.

A preferred compound of formula (I) of this invention is that wherein R¹ and R² independently represents a hydrogen atom, a fluorine atom, a chlorine atom, or an alkyl group having from 1 to 4 carbon atoms. Most preferably R¹ and R² independently represent a hydrogen atom, a fluorine atom or an alkyl group having from 1 to 3 carbon atoms.

A preferred compound of formula (I) of this invention is that wherein R³ represents an aryl group having from 6 to 7 ring carbon atoms or a heteroaryl group having from 5 to 10 ring atoms which consists of from 1 to 2 heteroatoms independently selected from the group consisting of sulfur atoms, oxygen atoms and nitrogen atoms. More preferably, R³ represents a phenyl group, a thiazolyl group, an isothiazolyl group, an oxazolyl group, an isoxazolyl group, a pyrrolyl group, a pyridyl group, a pyrimidine group, a quinolyl group, an isoquinolyl group, a tetrahydroquinolyl group, a tetrahydroisoquinolyl group, a chromanyl group or an isochromanyl group. Most preferably, R³ represents a phenyl group, a thiazolyl group, a pyridyl group, or an isochromanyl group. R³ is preferably unsubstituted or substituted by one or two a groups, preferably in the meta and/or para position relative to the point of attachment to the piperidyl ring. When R³ is phenyl, it is preferably substituted by one a group, preferably halogen atoms, alkoxy groups having from 1 to 6 carbon atoms or alkoxyalkyl groups having from 1 to 6 carbon atoms. When R¹ is monocyclic heteroaryl, it is preferably substituted by one or two a groups, most preferably one, preferably halogen atoms, alkoxy groups having from 1 to 6 carbon atoms or alkoxyalkyl groups having from 1 to 6 carbon atoms. When R³ is 3-pyridyl, it is preferably substituted by 6-alkoxy groups having from 1 to 6 carbon atoms.

A preferred individual compound of this invention is selected from
1-[2-(3-Fluoro-4-hydroxyphenyl)-2-hydroxyethyl]-4-(6-methoxypyridin-3-yl)piperidin-4-ol methanesulfonate;
4-(3,4-Dihydro-1H-isochromen-7-yl)-1-[2-(3-fluoro-4-hydroxyphenyl)-2-hydroxyethyl]piperidin-4-ol methanesulfonate;
1-[2-(3-Fluoro-4-hydroxyphenyl)-2-hydroxyethyl]-4-(3-fluorophenyl)piperidin-4-ol methanesulfonate;
4-(3,4-Dihydro-1H-isochromen-7-yl)-1-[2-hydroxy-2-(4-hydroxy-3-methylphenyl)ethyl]piperidin-4-ol;
4-(3-Fluorophenyl)-1-[2-hydroxy-2-(4-hydroxy-3-methylphenyl)ethyl]piperidin-4-ol;
1-[2-Hydroxy-2-(4-hydroxy-3-methylphenyl)ethyl]-4-(6-methoxypyridin-3-yl)piperidin-4-ol;
1-[2-(2-Fluoro-4-hydroxyphenyl)-2-hydroxyethyl]-4-(3-fluorophenyl)piperidin-4-ol;
4-(3,4-Dihydro-1H-isochromen-7-yl)-1-[2-(2-fluoro-4-hydroxyphenyl)-2-hydroxyethyl]piperidin-4-ol;
1-[2-(2-Fluoro-4-hydroxyphenyl)-2-hydroxyethyl]-4-(6-methoxypyridin-3-yl)piperidin-4-ol;
4-(3-Fluorophenyl)-1-[2-hydroxy-2-(4-hydroxyphenyl)ethyl]piperidin-4-ol;
1-[2-hydroxy-2-(4-hydroxyphenyl)ethyl]-4-(6-methoxypyridin-3-yl)piperidin-4-ol;
1-[2-Hydroxy-2-(4-hydroxyphenyl)ethyl]-4-[4-(methoxymethyl)phenyl]piperidin-4-ol;
1-[2-Hydroxy-2-(4-hydroxy-3-methylphenyl)ethyl]-4-[4-(methoxymethyl)phenyl]piperidin-4-ol;
1-[2-Hydroxy-2-(4-hydroxy-3-methylphenyl)ethyl]-4-(5-methyl-1,3-thiazol-2-yl)piperidin-4-ol;
1-[2-Hydroxy-2-(4-hydroxy-3-methylphenyl)ethyl]-4-(3-methoxyphenyl)-piperidin-4-ol hydrochloride;
4-(6-Ethoxypyridin-3-yl)-1-[2-hydroxy-2-(4-hydroxy-3-methylphenyl)ethyl]piperidin-4-ol;
1-[2-(2-Fluoro-4-hydroxy-5-methylphenyl)-2-hydroxyethyl]-4-(6-methoxypyridin-3-yl)piperidin-4-ol;
4-(6-Fluoro-5-methoxypyridin-2-yl)-1-[2-hydroxy-2-(4-hydroxy-3-methylphenyl)ethyl]piperidin-4-ol;
1-[2-(3-chloro-4-hydroxyphenyl)-2-hydroxyethyl]-4-(6-methoxypyridin-3-yl)piperidin-4-ol hydrochloride;
1-[2-(3-chloro-4-hydroxyphenyl)-2-hydroxyethyl]-4-[4-(methoxymethyl)phenyl]piperidin-4-ol;
1-[2-(2,5-difluoro-4-hydroxyphenyl)-2-hydroxyethyl]-4-(3-fluorophenyl)piperidin-4-ol; and
1-[2-(2,5-difluoro-4-hydroxyphenyl)-2-hydroxyethyl]-4-(6-methoxypyridin-3-yl)piperidin-4-ol; or a pharmaceutically acceptable salt thereof.

A further preferred individual compound of this invention is selected from
1-[2-(3-Fluoro-4-hydroxyphenyl)-2-hydroxyethyl]-4-(3-fluorophenyl)piperidin-4-ol methanesulfonate;
4-(3,4-Dihydro-1H-isochromen-7-yl)-1-[2-hydroxy-2-(4-hydroxy-3-methylphenyl)ethyl]piperidin-4-ol;
4-(3-Fluorophenyl)-1-[2-hydroxy-2-(4-hydroxy-3-methylphenyl)ethyl]piperidin-4-ol;
1-[2-Hydroxy-2-(4-hydroxy-3-methylphenyl)ethyl]-4-(6-methoxypyridin-3-yl)piperidin-4-ol;
1-[2-(2-Fluoro-4-hydroxyphenyl)-2-hydroxyethyl]-4-(3-fluorophenyl)piperidin-4-ol;
4-(3,4-Dihydro-1H-isochromen-7-yl)-1-[2-(2-fluoro-4-hydroxyphenyl)-2-hydroxyethyl]piperidin-4-ol;
4-(3-Fluorophenyl)-1-[2-hydroxy-2-(4-hydroxyphenyl)ethyl]piperidin-4-ol;
1-[2-hydroxy-2-(4-hydroxyphenyl)ethyl]-4-(6-methoxypyridin-3-yl)piperidin-4-ol;
1-[2-Hydroxy-2-(4-hydroxyphenyl)ethyl]-[4-(methoxymethyl)phenyl]piperidin-4-ol;
1-[2-Hydroxy-2-(4-hydroxy-3-methylphenyl)ethyl]-4-(3-methoxyphenyl)-piperidin-4-ol hydrochloride;
4-(6-Ethoxypyridin-3-yl)-1-[2-hydroxy-2-(4-hydroxy-3-methylphenyl)ethyl]piperidin-4-ol;
1-[2-(2-Fluoro-4-hydroxy-5-methylphenyl)-2-hydroxyethyl]-4-(6-methoxypyridin-3-yl)piperidin-4-ol; and
4-(6-Fluoro-5-methoxypyridin-2-yl)-1-[2-hydroxy-2-(4-hydroxy-3-methylphenyl)ethyl]piperidin-4-ol;
or a pharmaceutically acceptable salt thereof.

### General Synthesis

The compounds of the present invention may be prepared by a variety of processes well known for the preparation of compounds of this type, for example as shown in the following reaction Schemes. Unless otherwise indicated R¹, R² and R³ in the reaction Schemes and discussion that follow are defined as above. The term “protecting group”, as used hereinafter, means a hydroxy or amino protecting group which is selected from typical hydroxy or amino protecting groups described in Protective Groups in Organic Synthesis edited by T. W. Greene et al. (John Wiley & Sons, 1991);

The following reaction Schemes illustrate the preparation of compounds of formula (I).

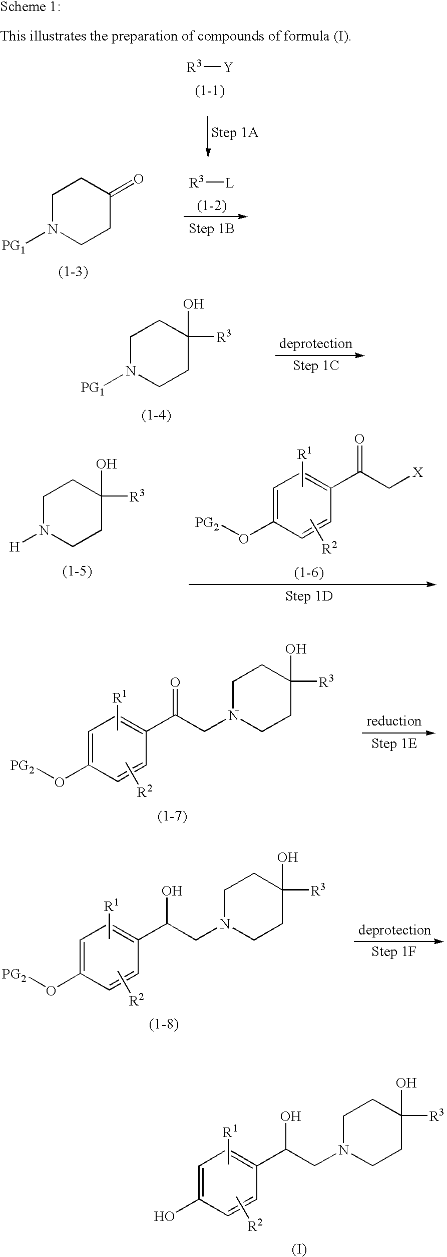

In the above formula, X represents a leaving group. Example of suitable leaving groups include: halogen atoms, such as chlorine, bromine and iodine; sulfonic esters such as TfO (triflates), MsO (mesylates), TsO (tosylates); and the like. Y represents a hydrogen atom, a halogen atom such as, fluorine, chlorine, bromine or iodine; .L represents metal such as lithium, or MgY. PG¹ and PG² independently represents a protecting group. The term “protecting group”, as used herein, means a hydroxy or amino protecting group which is selected from typical hydroxy or amino protecting groups described in Protective Groups in Organic Synthesis edited by T. W. Greene et al. (John Wiley & Sons, 1991). Typical hydroxy or amino protecting groups include benzyl, C₂H₅O(C═O)—, CH₃(C═O)—, t-butyldimethylsilyl (TBS), t-butyldiphenylsilyl, triisopropylsilyl (TIPS), methoxymethyl (MOM), benzyloxycarbonyl represented as Z and t-buthoxycarbonyl represented as t-Boc or Boc.

Step 1A

In this Step, the organometallic compound of formula (1-2) can be prepared by reaction of a halide compound of formula (1-1). This reaction may be carried out in the presence of an organometallic reagent or a metal. Examples of suitable organometallic reagents include; alkyllithiums such as n-butyllithium, sec-butyllithium and tert-butyllithium; aryllithiums such as phenyllithium and lithium naphtilide. Examples of suitable metal include magnesium. Preferred reaction inert solvents include, for example, hydrocarbons, such as hexane; ethers, such as diethyl ether, diisopropyl ether, dimethoxyethane (DME) tetrahydrofuran (THF) and dioxane; or mixtures thereof. Reaction temperatures are generally in the range of −100 to 50° C., preferably in the range of from −100° C. to room temperature. Reaction times are, in general, from 1 minute to a day, preferably from 1 hour to 10 hours.

Step 1B

In this Step, an alcohol compound of formula (1-4) can be prepared by the nucleophilic addition of a ketone compound of formula (1-1) with the organometallic compound of formula (1-2). The reaction may be carried out in the presence of a solvent. Examples of suitable solvents include for example, hydrocarbons, such as hexane; ethers, such as diethyl ether, diisopropyl ether, dimethoxyethane (DME) tetrahydrofuran (THF) and dioxane; or mixtures thereof. Reaction temperatures are generally in the range of −100 to 50° C., preferably in the range of from −100° C. to room temperature. Reaction times are, in general, from 1 minute to a day, preferably from 1 hour to 10 hours.

Step 1C

In this Step, the desired compound of formula (1-5) may be prepared by the deprotection of the compound of formula 1-4, prepared as described in Step 1B, according to known procedures such as those described in Protective Groups in Organic Synthesis edited by T. W. Greene et al. (John Wiley & Sons, 1991).

In the case of Boc protection, the removal of the protecting groups may be carried out under known conditions in the presence or the absence of catalytic amount of an acid in a reaction inert solvent. Example of suitable aqueous or non-aqueous organic reaction inert solvents include: ethyl acetate; alcohols, such as methanol and ethanol; ethers, such as tetrahydrofuran and dioxane; acetone; dimethylformamide; halogenated hydrocarbons, such as dichloromethane, dichloroethane or chloroform; and acetic acid or mixtures thereof. The reaction may be carried out at a temperature in the range from of 0° C. to 200° C., preferably in the range of 20° C. to 120° C. Reaction times are, in general, from 1 minute to 48 hours, preferably 5 minutes to 24 hours. Example of suitable catalysts include: hydrogen halide, such as hydrogen chloride and hydrogen bromide; sulfonic acids, such as p-toluenesulfonic acid and, benzenesulfonic acid; ammonium salts, such as pyridium p-toluenesulfonate and ammonium chloride; and carboxylic acid, such as acetic acid and trifluoroacetic acid.

In the case of Bn or Z protection, the removal of the protecting groups may be carried out under, for example, known hydrogenolysis conditions in the presence of a metal catalyst under hydrogen atmosphere or in the presence of hydrogen sources such as formic acid or ammonium formate in a reaction inert solvent. If desired, the reaction is carried out under acidic conditions, for example, in the presence of hydrochloric acid or acetic acid. A preferred metal catalyst is selected from, for example, palladium-carbon, palladiumhydroxide-carbon, platinumoxide, platinum-carbon, ruthenium-carbon, rhodium-aluminumoxide, tris[triphenyphosphine]rhodiumchlrodie. Example of suitable reaction inert aqueous or non-aqueous organic solvents include: alcohols, such as methanol, ethanol; ethers, such as tetrahydrofuran or dioxane; acetone; dimethylformamide; halogenated hydrocarbons, such as dichloromethane, dichloroethane or chloroform; and acetic acid or mixtures thereof The reaction may be carried out at a temperature in the range from of 20° C. to 100° C., preferably in the range of 20° C. to 60° C. Reaction times are, in general, from 10 minutes to 48 hours, preferably 30 minutes to 24 hours. This reaction may be carried out under hydrogen atmosphere at a pressure ranging from 1 to 100 atom, preferably from 1 to 10 atom.

In the case of ethoxycarbonyl protection, the removal of the protecting groups may be carried out under known conditions. In a typical procedure, this reaction can be carried out by treatment with sodium hydroxide, lithium hydroxide, trimethylsilyl iodide or alkylthiolithium such as n-propylthiolithium in a reaction inert solvent. Suitable solvents include, for example, alcohols such as methanol, ethanol, propanol, butanol, 2-methoxyethanol, and ethlene gylcol; ethers such as tetrahydrofuran (THF), 1,2-dimethoxyethane (DME), and 1,4-dioxane; halogenated hydrocarbons such as chloroform, dichloroethane, and 1,2-dichloroethane; amides such as N,N-dimethylformamide (DMF) and hexamethylphospholictriamide; and sulfoxides such as dimethyl sulfoxide (DMSO). This reaction may be carried out at a temperature in the range from −10 to 200° C., usually from 0° C. to 120° C. for 30 minutes to 24 hours, usually 60 minutes to 10 hour.

Step 1D

In this Step, the desired beta-carbonyl piperidne compound of formula 1-7 may be prepared by the coupling of a halide compound of formula 1-6 with the piperidine compound of formula 1-5 in an inert solvent, e.g. aliphatic hydrocarbons, such as hexane, heptane and petroleum ether; aromatic hydrocarbons, such as benzene, toluene, xylene and nitrobenzene; halogenated hydrocarbons, such as methylene chloride, chloroform, carbon tetrachloride and dichloroethane; ethers, such as diethyl ether, diisopropyl ether, tetrahydrofuran and dioxane; alcohols, such as methanol, ethanol, propanol, isopropanol and butanol; and dimethylformamide (DMF), dimethylsulfoxide (DMSO), 1,3-dimethyl-2-imidazolidinone (DMI) or acetonitrile. This reaction may be carried out in the presence of a base, e.g. an alkali or alkaline earth metal hydroxide, alkoxide, carbonate, or hydride, such as sodium hydroxide, potassium hydroxide, sodium methoxide, sodium ethoxide, potassium tert-butoxide, sodium carbonate, potassium carbonate, cesium carbonate, sodium hydride or potassium hydride, or an amine such as triethylamine, tributylamine, diisopropylethylamine, pyridine or dimethylaminopyridine. This reaction may be carried out in the presence of a suitable additive, e.g. tetrakis(triphenylphosphine)-palladium, bis(triphenylphosphine)palladium(II) chloride, copper(0), copper(I) acetate, copper(I) bromide, copper(I) chloride, copper(I) iodide, copper(I) oxide, copper(I) trifluoromethanesulfonate, copper(II) acetate, copper(I) bromide, copper(II) chloride, copper(II) iodide, copper(II) oxide, 1,10-phenanthroline, dibenzanthracene (DBA) or copper(II) trifluoromethanesulfonate. The reaction may be carried out at a temperature in the range from of 0° C. to 100° C., preferably in the range of 20° C. to 100° C. Reaction times are, in general, from 5 minutes to 48 hours, preferably 30 minutes to 24 hours.

Step 1E

In this Step, an alcohol compound of formula (1-8) can be prepared by the reduction of the ketone compound of formula (1-7) with a reducing agent, e.g. NaBH₄, LiAlH₄, LiBH₄, or ZnBH₄ in an inert solvent, e.g. methanol, ethanol, diglyme, or mixtures thereof. The reaction may be carried out at a temperature in the range from of 0° C. to 100° C., preferably in the range of 20° C. to 80° C. Reaction times are, in general, from 5 minutes to 48 hours, preferably 30 minutes to 24 hours.

Step 1F

In this Step, the desired compound of formula (I) may be prepared by the deprotection of the compound of formula (1-8).

This reaction is essentially the same as and may be carried out in the same manner as and using the same reagents and reaction conditions as Step 1C in Scheme 1.

In the case of silyl derivatives protection, the removal of the protecting groups may be carried out under known conditions. In a typical procedure, this reaction can be carried out by treatment with tetrabutylammonium fluoride in tetrahydrofuran. This reaction can be also carried out under the acidic conditions in a reaction inert solvent. Example of suitable aqueous or non-aqueous organic reaction inert solvents include: alcohols, such as methanol and ethanol; ethers, such as tetrahydrofuran and dioxane; acetone; dimethylformamide; and acetic acid or mixtures thereof. The reaction may be carried out at a temperature in the range from of −10° C. to 200° C., preferably in the range of 0C to 120° C. Reaction times are, in general, from 1 minute to 48 hours, preferably 5 minutes to 24 hours. Example of suitable acids include: hydrogen halide, such as hydrogen chloride and hydrogen bromide; sulfonic acids, such as p-toluenesulfonic acid and, benzenesulfonic acid; ammonium salts, such as pyridium p-toluenesulfonate and ammonium chloride; and carboxylic acid, such as acetic acid and trifluoroacetic acid. 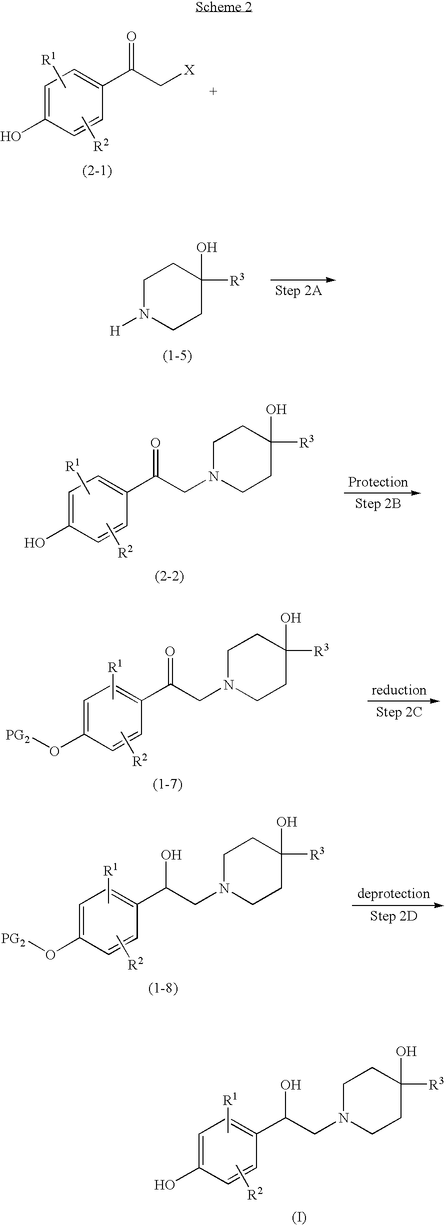
Step 2A

In this Step, the desired beta-carbonyl piperidne compound of formula 2-2 may be prepared by the coupling of a halide compound of formula 2-1 with the piperidine compound of formula 1-5. This reaction is essentially the same as and may be carried out in the same manner as and using the same reagents and reaction conditions as Step 1D in Scheme 1.

Step 2B

In this Step, the protected compound of formula (1-7) may be prepared from the compound of formula (2-2) by converting the OH group into a protected O group. The step may be carried out by using, for example, the compound of formula (2-2), appropriate triethyl orthoformate, silyl halides, aralkyl halide, acid halides, acid anhydride and acids, such as benzyl, t-butyldimethylsilyl (TBS) chloride, t-butyldiphenylsilylchloride, Z-chloride and t-BocCl or Boc₂O, using the methods described in Protective Groups in Organic Synthesis edited by T. W. Greene et al. (John Wiley & Sons, 1991). Of these reagents, we prefer triethyl orthoformate. The reaction may be carried out in the presence or absence of a solvent, e.g. aromatic hydrocarbons, such as benzene, toluene and xylene; halogenated hydrocarbons, such as methylene chloride, chloroform, carbon tetrachloride and dichloroethane; and ethers, such as diethyl ether, diisopropyl ether, tetrahydrofuran and dioxane; and DMF and DMSO. This reaction may be carried out in the presence or absence of a catalyst, e.g. para-toluenesulfonic acid, camphorsulfonic acid, and acetic acid.

Step 2C and 2D

In these Steps, the desired compound of formula (I) may be prepared by the reduction of the ketone compound of formula (1-7) followed by the deprotection of the compound of formula (1-8).

These reactions are essentially the same as and may be carried out in the same manner as and using the same reagents and reaction conditions as Step 1E and 1F in Scheme 1.

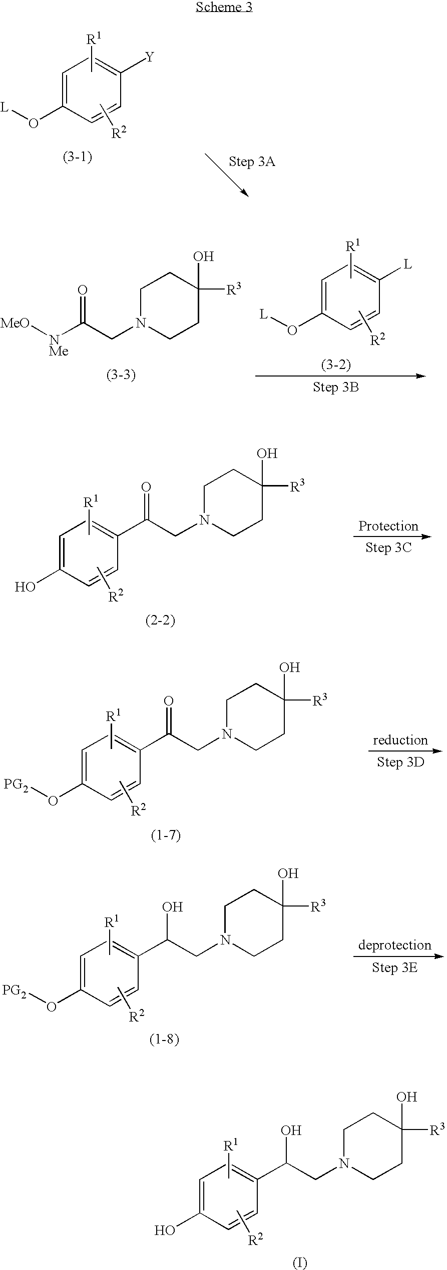

In the above formula, Y represents a halogen atom such as, fluorine, chlorine, bromine or iodine; .L represents metal such as lithium, or MgY.

Step 3A

In this Step, the organometallic compound of formula (3-2) can be prepared by reaction of a halide compound of formula (3-1). This reaction may be carried out in the presence of an organometallic reagent or a metal. Examples of suitable organometallic reagents include; alkyllithiums such as n-butyllithium, sec-butyllithium and tert-butyllithium; aryrlithiums such as phenyllithium and lithium naphtilide. Examples of suitable metal include magnesium. Preferred reaction inert solvents include, for example, hydrocarbons, such as hexane; ethers, such as diethyl ether, diisopropyl ether, dimethoxyethane (DME) tetrahydrofuran (THF) and dioxane; or mixtures thereof. Reaction temperatures are generally in the range of −100 to 50° C., preferably in the range of from −100° C. to room temperature. Reaction times are, in general, from 1 minute to a day, preferably from 1 hour to 10 hours.

Step 3B

In this Step, the desired beta-carbonyl piperidine compound of formula 2-2 may be prepared by the coupling of the amide compound of formula 3-2 with a Weinreb amide compound of formula 3-3. The reaction may be carried out in the presence of a solvent. Examples of suitable solvents include for example, hydrocarbons, such as hexane; ethers, such as diethyl ether, diisopropyl ether, dimethoxyethane (DME) tetrahydrofuran (THF) and dioxane; or mixtures thereof. Reaction temperatures are generally in the range of −100 to 50° C., preferably in the range of from −100° C. to room temperature. Reaction times are, in general, from 1 minute to a day, preferably from 1 hour to 10 hours.

Step 3C

In this Step, the protected compound of formula (1-7) may be prepared from the compound of formula (2-2) by converting the OH group into a protected O group.

These reactions are essentially the same as and may be carried out in the same manner as and using the same reagents and reaction conditions as Step 1E and 2B in Scheme 2.

Step 3D and 3E

In these Steps, the desired compound of formula (I) may be prepared by the reduction of the ketone compound of formula (1-7) followed by the deprotection of the compound of formula (1-8).

These reactions are essentially the same as and may be carried out in the same manner as and using the same reagents and reaction conditions as Step 1E and 1F in Scheme 1. 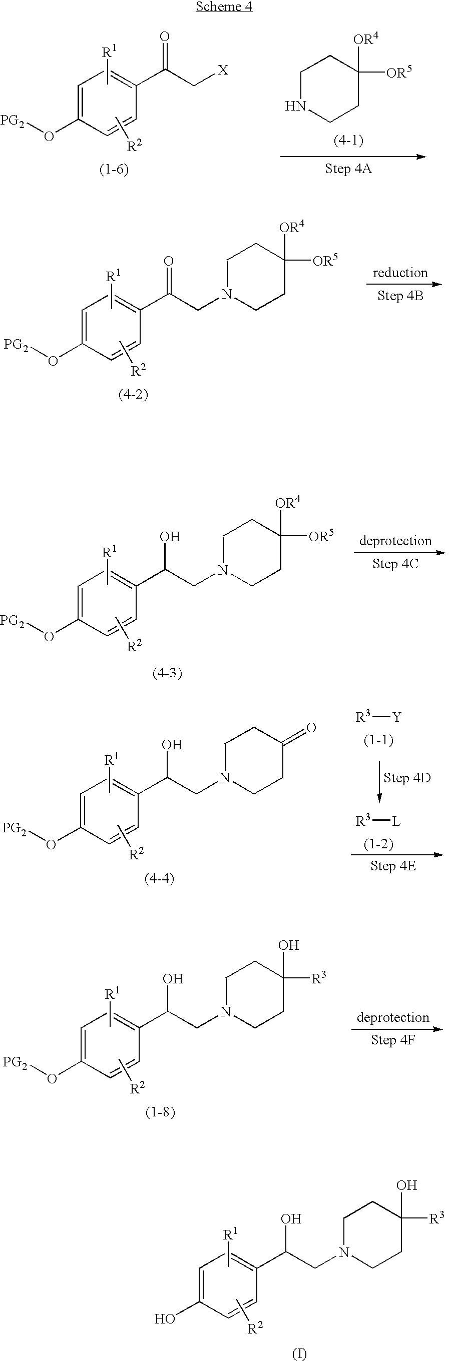
Step 4D

In the above formula, R4 and R5 represents an alkyl group or R4 and R5 may be joined together to form an ethylene or a propylene group; said ethylene or propylene group are optionally substituted by hydroxy groups.

Step 4A

In this Step, a desired beta-carbonyl piperidne compound of formula 4-2 may be prepared by the coupling of a halide compound of formula 1-6 with an ketal piperidine compound of formula 4-1. This reaction is essentially the same as and may be carried out in the same manner as and using the same reagents and reaction conditions as Step 1D in Scheme 1.

Step 4B

In this Step, an alcohol compound of formula (4-3) can be prepared by the reduction of the ketone compound of formula (4-2) with a reducing agent. This reaction is essentially the same as and may be carried out in the same manner as and using the same reagents and reaction conditions as Step 1E in Scheme 1.

Step 4C

In this Step, a piperidone compound of formula (4-4) can be prepared by the deprotection of the ketal compound of formula (4-3) in the presence or the absence of a catalyst in a reaction-inert solvent.

The hydrolysis reaction may be carried out in an aqueous or non-aqueous organic solvent. Examples of suitable solvents include: alcohols, such as methanol or ethanol; ethers, such as tetrahydrofuran or dioxane; acetone; dimethylformamide; halogenated hydrocarbons, such as dichloromethane, dichloroethane or chloroform; acids, such as acetic acid, hydrogen chloride, hydrogen bromide and sulfuric acid. Example of suitable catalysts include: hydrogen halides, such as hydrogen chloride and hydrogen bromide; sulfonic acids, such as p-toluenesulfonic acid and benzenesulfonic acid; ammonium salts, such as pyridium p-toluenesulfonate and ammonium chloride; and carboxylic acid, such as acetic acid and trifluoroacetic acid. This reaction can be carried out at temperature of 0° C. to 200° C., preferably from about 20° C. to 120° C. for 5 minutes to 48 hours, preferably 30 minutes to 24 hours.

Step 4D

In this Step, the organometallic compound of formula (1-2) can be prepared by reaction of a halide compound of formula (1-1) in the same manner as and using the same reagents and reaction conditions as Step 1A in Scheme 1.

Step 4E

In this Step, the alcohol compound of formula (1-8) can be prepared by the nucleophilic addition of the ketone compound of formula (4-4) with the organometallic compound of formula (1-2). This reaction is essentially the same as and may be carried out in the same manner as and using the same reagents and reaction conditions as Step 1B in Scheme 1.

Step 4F

In this Step, the desired compound of formula (I) may be prepared by the deprotection of the compound of formula (1-8).

This reaction is essentially the same as and may be carried out in the same manner as and using the same reagents and reaction conditions as Step 1C or 1F in Scheme 1.

The starting materials in the aforementioned general syntheses may be commercially available or obtained by conventional methods known to those skilled in the art.

In the above Schemes from 1 to 4, examples of suitable solvents include a mixture of any two or more of those solvents described in each Step.

The compounds of formula (I), and the intermediates above-mentioned preparation methods can be isolated and purified by conventional procedures, such as recrystallization or chromatographic purification.

The optically active compounds of this invention can be prepared by several methods. For example, the optically active compounds of this invention may be obtained by chromatographic separation, enzymatic resolution or fractional crystallization from the final compounds.

### Method for Assessing Biological Activities

NR2B Binding Assay

The activity of the bicyclic amide compounds of the present invention, as NR2B antagonists, is determined by their ability to inhibit the binding of NR2B subunit at its receptor sites employing radioactive ligands.

The NR2B antagonist activity of the bicyclic amide compounds is evaluated by using the standard assay procedure described in, for example, J. Pharmacol., 331, pp 117-126, 1997. This method essentially involves determining the concentration of the individual compound required to reduce the amount of radiolabelled NR2B ligands by 50% at their receptor sites, thereby affording characteristic IC₅₀ values for each compound tested. More specifically, the assay is carried out as follows.

Membranes were prepared by homogenization of forebrain of male CD rats weighing between 170˜190 g by using glass-Teflon homogenizer in 0.32 M sucrose at 4° C. The crude nuclear pellet was removed by centrifugation at 1000×g for 10 min, and the supernatant centrifuged at 17000×g for 25 min. The resulting pellet was resuspended in 5 mM Tris acetate pH 7.4 at 4° C. for 10 min to lyse cellular particles and again centrifuged at 17000×g. The resulting pellet (P2 membrane) was washed twice in Tris acetate, resuspended at 5.5 mg protein/ml and stored at −20° C. until use. All the manipulation was done on ice, and stock solution and equipment were kept on ice at all time.

For the saturation assay, receptor saturation was determined by incubating [³H]-CP-98,113 and 50 μg protein of P2 membrane for 60 minutes at room temperature in a final 100 μl of incubation buffer (50 mM Tris HCl, pH7.4). Total and non-specific bindings (in the presence of 10 μM of unlabeled CP-98,113) were determined in a range of [³H]-CP-98113 concentrations (0.625 nM to 60 nM). [³H]-CP-98,113 is as follows:

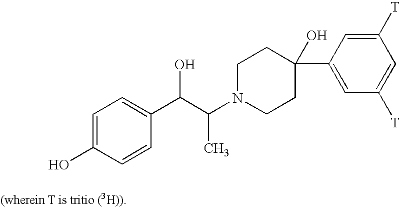

For the competition assay, test compounds were incubated in duplicate with 5 nM [³C]-CP-98,113 and 50 μg protein of P2 membrane for 60 minutes at room temperature in a final 100 μl of 50 mM Tris HCl buffer (pH7.4). Nonspecific binding was determined by 10 μM of unlabeled CP-98,113 (25 μl). The saturation derived K_{D} gained in saturation assay was used for all Ki calculations.

All incubations were terminated by rapid vacuum filtration over 0.2% polyethyleneimine soaked Whatman GF/B glass fibre filter paper using a SKATRON cell harvester followed by three washes with ice-cold filtration buffer (5 mM Tris HCl, pH 7.4.). Receptor-bound radioactivity was quantified by liquid scintillation counting using Packard LS counter. Competition assays were performed by counting Wallac GF/B filters on Betaplate scintillation counter (Wallac).

AU compounds prepared in the working examples as described below were tested by this method, and they showed Ki values from 2 nM to 20 nM with respect to inhibition of binding at the NR2B receptor.

Human NR2B Cell Functional Assay

HEK293 cells stably expressing human NR1b/2B receptor were used for cell functional assay. Cells were grown in 75-cm² culture flasks, using Dulbecco's modified Eagle's medium (DMEM, high glucose) supplemented with 10% fetal bovine, 52 μg/ml Zeocin, 530 μg/ml Geneticin, 100 units/ml penicillin and 100 μg/ml streptomycin. Cells were maintained in a humidified atmosphere in 5% CO₂ at 37° C., and 50-60% confluent cells were harvested by 0.05% trypsin containing 0.53 mM EDTA. The day before the experiment, expression of NR1b/2B receptor was induced by 5 μM ponasteron A in DMEM (40 ml) in the presence of 400 μM ketamine to prevent excitotoxicity. The induction was performed for 19-24 hours, using 50-60% confluent cells.

Cells were washed with 10 ml of Ca²⁺-free Krebs-Ringer Hepes buffer (KRH) containing 400 μM ketamine, and the loading of 5 μM fura-2 acetoxymethyl ester was made for 2 hrs at room temperature in the presence of 400 μM ketamine in Ca²⁺-free KRH (10 ml). Subsequently, cells were collected in 50 ml tube by pipetting manipulation and centrifuged at 850 rpm for 2 min. Supernatant was removed, and cells were washed with 10 ml of Ca²⁺-free KRH buffer, followed by centrifugation again. This manipulation was repeated 4 times to remove ketamine, glutamate and glycine. Cells were re-suspended in Ca²⁺-free KRH buffer, and 50 μl of cell suspension was added to each well of 96-well plates at a density of 100,000 cells/well, followed by adding test compounds dissolved in 50 μl of Ca²⁺-free KRH. After pre-incubation for 30 min, agonists (final 100 μM glutamic acid and 10 μM glycine) dissolved in 25 μl of KRH containing 9 mM Ca²⁺ (final 1.8 mM) were added. Fura-2 fluorescence (excitation wavelengths: 340 nm and 380 nm; emission wavelengths 510-520 nm) was monitored with a fluorescence imaging system, FDSS6000. The Δ fluorescence ratio F340/F380 (i.e., the fluorescence ratio immediately post-agonist—the basal fluorescence ratio; calculated as AUC) was used for evaluation of drug effects on agonists-induced changes in intracellular Ca²⁺. The basal fluorescence ratio was determined in the presence of 10 μM MK-801.

Rat Haloperidol-Induced Catalepsy Assay:

Fasted male CD rats were used (7-8 weeks old). Test compound or vehicle was given subcutaneously then haloperidol 0.5 mg/kg s.c. Sixty minutes after haloperidol-injection, the duration of catalepsy was quantified by placing the animals forepaws on an elevated bar and determining the latency to remove both forepaws from the bar. The cutoff latency was 60 seconds. Experimenter was blind to treatments during testing.

Human Dofetilide Binding

Human BERG transfected HEK293S cells were prepared and grown in-house. The collected cells were suspended in 50 mM Tris-HCl (pH 7.4 at 4° C.) and homogenized using a hand held Polytron PT 1200 disruptor set at full power for 20 sec on ice. The homogenates were centrifuged at 48,000×g at 4° C. for 20 min. The pellets were then resuspended, homogenized, and centrifuged once more in the same manner. The final pellets were resuspended in an appropriate volume of 50 mM Tris-HCl, 10 mM KCl, 1 mM MgCl₂ (pH 7.4 at 4° C.), homogenized, aliquoted and stored at −80° C. until use. An aliquot of membrane fractions was used for protein concentration determination using BCA protein assay kit (PIERCE) and ARVOsx plate reader (Wallac).

Binding assays were conducted in a total volume of 200 μl in 96-well plates. Twenty μl of test compounds were incubated with 20 μl of [³H]-dofetilide (Amersham, final 5 nM) and 160 μl of membrane homogenate (25 μg protein) for 60 minutes at room temperature. Nonspecific binding was determined by 10 μM dofetilide at the final concentration. Incubation was terminated by rapid vacuum filtration over 0.5% presoaked GF/B Betaplate filter using Skatron cell harvester with 50 mM Tris-HCl, 10 mM KCl, 1 mM MgCl₂, pH 7.4 at 4° C. The filters were dried, put into sample bags and filled with Betaplate Scint. Radioactivity bound to filter was counted with Wallac Betaplate counter.

All compounds prepared in the working examples as described below showed a TI (TI is a value of {Dofetilide Binding Ki [μM]/NR2B Binding Ki [nM]×1000} value in the range of 500-3800, whereas a structurally similar comparative compound A showed a TI value of 220.

I_{HERG} Assay

HEK 293 cells which stably express the HERG potassium channel were used for electrophysiological study. The methodology for stable transfection of this channel in HEK cells can be found elsewhere (Z. Zhou et al., 1998, Biophysical journal, 74, pp230-241). Before the day of experimentation, the cells were harvested from culture flasks and plated onto glass coverslips in a standard MEM medium with 10% FCS. The plated cells were stored in an incubator at 37° C. maintained in an atmosphere of 95% O₂/5% CO₂. Cells were studied between 15-28 hrs after harvest.

HERG currents were studied using standard patch clamp techniques in the whole-cell mode. During the experiment the cells were superfused with a standard external solution of the following composition (mM); NaCl, 130; KCl, 4; CaCl₂, 2; MgCl₂, 1; Glucose, 10; HEPES, 5; pH 7.4 with NaOH. Whole-cell recordings was made using a patch clamp amplifier and patch pipettes which have a resistance of 1-3 MOhm when filled with the standard internal solution of the following composition (mM); KCl, 130; MgATP, 5; MgCl₂, 1.0; HEPES, 10; EGTA 5, pH 7.2 with KOH. Only those cells with access resistances below 15 MΩ and seal resistances >1 GΩ was accepted for further experimentation. Series resistance compensation was applied up to a maximum of 80%. No leak subtraction was done. However, acceptable access resistance depended on the size of the recorded currents and the level of series resistance compensation that can safely be used. Following the achievement of whole cell configuration and sufficient for cell dialysis with pipette solution (>5 min), a standard voltage protocol was applied to the cell to evoke membrane currents. The voltage protocol is as follows. The membrane was depolarized from a holding potential of −80 mV to +20 mV for 1000 ms. This was followed by a descending voltage ramp (rate 0.5 mV msec⁻¹) back to the holding potential. The voltage protocol was applied to a cell continuously throughout the experiment every 4 seconds (0.25 Hz). The amplitude of the peak current elicited around −40 mV during the ramp was measured. Once stable evoked current responses were obtained in the external solution, vehicle (0.5% DMSO in the standard external solution) was applied for 10-20 min by a peristalic pump. Provided there were minimal changes in the amplitude of the evoked current response in the vehicle control condition, the test compound of either 0.3, 1, 3, 10 μM was applied for a 10 min period. The 10 min period included the time which supplying solution was passing through the tube from solution reservoir to the recording chamber via the pump. Exposing time of cells to the compound solution was more than 5 min after the drug concentration in the chamber well reached the attempting concentration. There reversibility. Finally, the cells was exposed to high dose of dofetilide (5 μM), a specific IKr blocker, to evaluate the insensitive endogenous current.

All experiments were performed at room temperature (23±1° C.). Evoked membrane currents were recorded on-line on a computer, filtered at 500-1 KHz (Bessel −3 dB) and sampled at 1-2 KHz using the patch clamp amplifier and a specific data analyzing software. Peak current amplitude, which occurred at around −40 mV, was measured off line on the computer.

The arithmetic mean of the ten values of amplitude was calculated under control conditions and in the presence of drug. Percent decrease of I_{N} in each experiment was obtained by the normalized current value using the following formula: I_{N}=(1-I_{D}/I_{C})×100, where I_{D} is the mean current value in the presence of drug and I_{C} is the mean current value under control conditions. Separate experiments were performed for each drug concentration or time-matched control, and arithmetic mean in each experiment is defined as the result of the study.

Mice PSL Method

Surgery of partial sciatic nerve ligation (PSL) was made according to Seltzer et al. (Pain 43, 1990, 205-218). Von Fray hair test was applied slowly to the plantar surface of the hind operated paw until the hairs bent. Each hair was tested 10 times in ascending order of force to different loci of the paw with one to two second intervals between each application. Once a withdrawal response was established, the paw was re-tested with the same hair. The lowest amount of force required to elicit a response was recorded as the paw-withdrawal threshold, measured in grams.

Chronic Contriction Injury Model (CCI Model):

Male Sprague-Dawley rats (270-300 g; B.W., Charles River, Tsukuba, Japan) were used.

The chronic constriction injury (CCI) operation was performed according to the method described by Bennett and Xie¹). Briefly, animals were anesthetized with sodium pentobarbital (64.8 mg/kg, i.p.) and the left common sciatic nerve was exposed at the level of the middle of the thigh by blunt dissection through biceps femoris. Proximal to the sciatic's trifurcation was freed of adhering tissue and 4-ligatures (4-0 silk) were tided loosely around it with about 1 mm space. Sham operation was performed as same as CCI surgery except for sciatic nerve ligation. Two weeks after surgery, mechanical allodynia was evaluated by application of von Frey hairs (VFHs) to the plantar surface of the hind paw. The lowest amount of force of VFH required to elicit a response was recorded as paw withdrawal threshold (PWT). VFH test was performed at 0.5, 1 and 2 hr post-dosing. Experimental data were analyzed using Kruskal-Wallis test followed by Dunn's test for multiple comparisons or Mann-Whitney U-test for paired comparison.
¹⁾ Bennett, G. J. and Xie, Y. K. {Pain, 33:87-107, 1988

Pharmaceutically acceptable salts of the compounds of formula (I) include the acid addition and base salts (including disalts) thereof.

Suitable acid addition salts are formed from acids which form non-toxic salts. Examples include the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, and tosylate salts. Suitable base salts are formed from bases which form non-toxic salts. Examples include the aluminium, arginine, benzathine, calcium, choline, diethylamine, diolamine, glycine, lysine, magnesium, meglumine, olanine, potassium, sodium, tromethamine and zinc salts.

For a review on suitable salts, see Stahl and Wermuth, Handbook of Pharmaceutical Salts: Properties, Selection, and Use, Wiley-VCH, Weinheim, Germany (2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent.

Pharmaceutically acceptable solvates in accordance with the invention include hydrates and solvates wherein the solvent of crystallization may be isotopically substituted, e.g. D₂O, d₆-acetone, d₆-DMSO.

Also within the scope of the invention are clathrates, drug-host inclusion complexes wherein, in contrast to the aforementioned solvates, the drug and host are present in non-stoichiometric amounts. For a review of such complexes, see J Pharm Sci, 64 (8), 1269-1288 by Haleblian (August 1975).

Hereinafter all references to compounds of formula (I) include references to salts thereof and to solvates and clathrates of compounds of formula (I) and salts thereof.

The invention includes all polymorphs of the compounds of formula (I) as hereinbefore defined.

Also within the scope of the invention are so-called “prodrugs” of the compounds of formula (I). Thus certain derivatives of compounds of formula (I) which have little or no pharmacological activity themselves can, when metabolised upon administration into or onto the body, give rise to compounds of formula (I) having the desired activity. Such derivatives are referred to as “prodrugs”.

Prodrugs in accordance with the invention can, for example, be produced by replacing appropriate functionalities present in the compounds of formula (I) with certain moieties known to those skilled in the art as “pro-moieties” as described, for example, in “Design of Prodrugs” by H Bundgaard (Elsevier, 1985).

Finally, certain compounds of formula (I) may themselves act as prodrugs of other compounds of formula (I).

Compounds of formula (I) containing one or more asymmetric carbon atoms can exist as two or more optical isomers. Where a compound of formula (I) contains an alkenyl or alkenylene group, geometric cis/trans (or Z/E) isomers are possible, and where the compound contains, for example, a keto or oxime group, tautomeric isomerism (‘tautomerism’) may occur. It follows that a single compound may exhibit more than one type of isomerism.

Included within the scope of the present invention are all optical isomers, geometric isomers and tautomeric forms of the compounds of formula (I), including compounds exhibiting more than one type of isomerism, and mixtures of one or more thereof.

Cis/trans isomers may be separated by conventional techniques well known to those skilled in the art, for example, fractional crystallization and chromatography.

Conventional techniques for the preparation/isolation of individual stereoisomers include the conversion of a suitable optically pure precursor, resolution of the racemate (or the racemate of a salt or derivative) using, for example, chiral HPLC, or fractional crystaliization of diastereoisomeric salts formed by reaction of the racemate with a suitable optically active acid or base, for example, tartaric acid.

The present invention also includes all pharmaceutically acceptable isotopic variations of a compound of formula (I). An isotopic variation is defined as one in which at least one atom is replaced by an atom having the same atomic number, but an atomic mass different from the atomic mass usually found in nature.

Examples of isotopes suitable for inclusion in the compounds of the invention include isotopes of hydrogen, such as ²H and ³H, carbon, such as ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution of the compounds of the invention with isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations of the compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variations of the compounds of formula (I) can generally be prepared by conventional techniques known to those skilled in the art or by processes analogous to those described in the accompanying Examples and Preparations using appropriate isotopic variations of suitable reagents.

The compounds of formula (I) may be freeze-dried, spray-dried, or evaporatively dried to provide a solid plug, powder, or film of crystalline or amorphous material. Microwave or radio frequency drying may be used for this purpose.

The compounds of the invention may be administered alone or in combination with other drugs and will generally be administered as a formulation in association with one or more pharmaceutically acceptable excipients. The term “excipient” is used herein to describe any ingredient other than the compound of the invention. The choice of excipient will to a large extent depend on the particular mode of administration.

The compounds of the invention may be administered in combination, separately, simultaneously or sequentially, with one or more other pharmacologically active agents. Suitable agents, particularly for the treatment of pain, include:
(i) opioid analgesics, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine and pentazocine;
(ii) nonsteroidal antiinflammatory drugs (NSAIDs), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, nabumetone, naproxen, oxaprozin, phenylbutazone, piroxicam, sulindac, tolmetin, zomepirac, and their pharmaceutically acceptable salts;
(iii) barbiturate sedatives, e.g. amobarbital, aprobarbital, butabarbital, butabital, mephobarbital, metharbital, methohexital, pentobarbital, phenobartital, secobarbital, talbutal, theamylal, thiopental and their pharmaceutically acceptable salts;
(iv) benzodiazepines having a sedative action, e.g. chlordiazepoxide, clorazepate, diazepam, flurazepar, lorazepam, oxazepam, temazepam, triazolam and their pharmaceutically acceptable salts,
(v) H₁ antagonists having a sedative action, e.g. diphenhydramine, pyrilamine, promethazine, chlorpheniramine, chlorcyclizine and their pharmaceutically acceptable salts;
(vi) miscellaneous sedatives such as glutethimide, meprobamate, methaqualone, dichloralphenazone and their pharmaceutically acceptable salts;
(vii) skeletal muscle relaxants, e.g. baclofen, carisoprodol, chlorzoxazone, cyclobenzaprine, methocarbamol, orphrenadine and their pharmaceutically acceptable salts,
(viii) alpha-2-delta ligands, e.g. gabapentin and pregabalin;
(ix) alpha-adrenergic active compounds, e.g. doxazosin, tamsulosin, clonidine and 4-amino-6,7-dimethoxy-2-(5-methanesulfonamido-1,2,3,4-tetrahydroisoquinol-2-yl)-5-(2-pyridyl)quinazoline;
(x) tricyclic antidepressants, e.g. desipramine, imipramine, amytriptiline and nortriptiline;
(xi) anticonvulsants, e.g. carbamazepine and valproate;
(xii) serotonin reuptake inhibitors, e.g. fluoxetine, paroxetine, citalopram and sertraline;
(xiii) mixed serotonin-noradrenaline reuptake inhibitors, e.g. milnacipran, venlafaxine and duloxetine;
(xiv) noradrenaline reuptake inhibitors , e.g. reboxetine;
(xv) Tachykinin (NK) antagonists, particularly Nk-3, NK-2 and NK-1 antagonists, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]methyl]-1,2-dihydro-3H-1,2,4-triazol-3-one (MK-869), lanepitant, dapitant and 3-[[2-methoxy-5-(trifluoromethoxy)phenyl]methylamino]-2-phenyl-piperidine (2S,3S)
(xvi) Muscarinic antagonists, e.g. oxybutin, tolterodine, propiverine, tropsium chloride and darifenacin;
(xvii) COX-2 inhibitors, e.g. celecoxib, rofecoxib and valdecoxib;
(xviii) Non-selective COX inhibitors (preferably with GI protection), e.g. nitroflurbiprofen (HCT-1026);
(xix) coal-tar analgesics, in particular, paracetamol;
(xx) neuroleptics, such as droperidol;
(xxi) Vanilloid receptor agonists, e.g. resinferatoxin;
(xxii) Beta-adrenergic compounds such as propranolol;
(xxiii) Local anaesthetics, such as mexiletine;
(xxiv) Corticosteriods, such as dexamethasone;
(xxv) serotonin receptor agonists and antagonists;
(xxvi) cholinergic (nicotinic) analgesics; and
(xxvii) miscellaneous analgesic agents, such as Tramadol®.

Thus, the invention further provides a combination comprising a compound of the invention or a pharmaceutically acceptable salt, solvate or pro-drug thereof, and a compound or class of compounds selected from the group (i)-(xxvii), above. There is also provided a pharmaceutical composition comprising such a combination, together with a pharmaceutically acceptable excipient, diluent or carrier, particularly for the treatment of a disease for which an alpha-2-delta ligand is implicated.

Combinations of the compounds of the present invention and other therapeutic agents may be administered separately, sequentially or simultaneously. Thus, the present invention extends to a kit comprising a compound of the invention, one or more other therapeutic agents, such as those listed above, and a suitable container.

The compounds of the present invention may be formulated by any convenient means using well-known carriers and excipients. Thus, the present invention also provides a pharmaceutical composition comprising a compound of the invention or a pharmaceutically acceptable ester or a pharmaceutically acceptable salt thereof with one or more pharmaceutically acceptable carriers.

Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents.

Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

The composition of a typical tablet in accordance with the invention may comprise:

| Ingredient | % w/w |
|---|---|
| Compound of formula (I) | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verna et al, Pharmaceutical Technology On-line, 25(2), 1-14 (2001). Other modified release formulations are described in U.S. Pat. No. 6,106,864.

Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

Topical Administration

The compounds of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus compounds of the invention may be formulated in a more solid form for administration as an implanted depot providing long-term release of the active compound.

Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate communicating method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 10 mg of the compound of the invention per actuation and the actuation volume may vary from 10 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as l-leucine, mannitol, or magnesium stearate.

In the case of dry powder inhalers and aerosols, the dosage unit is determined by means of a valve which delivers a metered amount. Units in accordance with the invention are typically arranged to administer a metered dose or “puff”.

Formulations for inhaled/intranasal administration may be formulated to be immediate and/or modified release. Modified release formulations include delayed-, sustained-, pulsed-, controlled dual-, targeted and programmed release.

Rectal/Vaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

Ocular/Andial Administration

The compounds of the invention may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

Enabling Technologies

The compounds of the invention may be combined with soluble macromolecular entities such as cyclodextrin or polyethylene glycol-containing polymers to improve their solubility, dissolution rate, taste-masking, bioavailability and/or stability. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Dosage

The compounds of the invention can be administered via either the oral, parenteral or topical routes to mammals. In general, these compounds are most desirably administered to humans in doses ranging from 0.1 mg to 3000 mg, preferably from 1 mg to 500 mg, which may be administered in a single dose or in divided doses throughout the day, although variations will necessarily occur depending upon the weight and condition of the subject being treated, the disease state being treated and the particular route of administration chosen.

These dosages are based on an average human subject having a weight of about 65 to 70 kg. The physician will readily be able to determine doses for subjects whose weight falls outside this range, such as infants and the elderly. For example, a dosage level that is in the range of from 0.01 mg to 10 mg per kg of body weight per day is most desirably employed for treatment of pain associated with inflammation.

### EXAMPLES

The invention is illustrated in the following non-limiting examples in which, unless stated otherwise: all operations were carried out at room or ambient temperature, that is, in the range of 18-25° C.; evaporation of solvent was carried out using a rotary evaporator under reduced pressure with a bath temperature of up to 60° C.; reactions were monitored by thin layer chromatography (tlc) and reaction times are given for illustration only; melting points (m.p.) given are uncorrected (polymorphism may result in different melting points); the structure and purity of all isolated compounds were assured by at least one of the following techniques: tlc (Merck silica gel 60 F₂₅₄s precoated TLC plates or Merck NH2 F₂₅₄, precoated HPTLC plates), mass spectrometry, nuclear magnetic resonance (NMR), infrared red absorption spectra (IR) or microanalysis. Yields are given for illustrative purposes only. Flash column chromatography was carried out using Merck silica gel 60 (230-400 mesh ASTM) or Fuji Silysia Chromatorexe® DU3050 (Amino Type, 30˜50 μm). Low-resolution mass spectral data (EI) were obtained on a Automass 120 (JEOL) mass spectrometer. Low-resolution mass spectral data (ESI) were obtained on a Quattro II (Micromass) mass spectrometer. Melting point was obtained using Seiko Instruments Inc. Exstar 6000. NMR data was determined at 270 MHz (JEOL JNM-LA 270 spectrometer) or 300 MHz (JEOL JNM-LA300) using deuterated chloroform (99.8% D) or dimethylsulfoxide (99.9% D) as solvent unless indicated otherwise, relative to tetramethylsilane (TMS) as internal standard in parts per million (ppm); conventional abbreviations used are: s=singlet, d=doublet, t=triplet, q=quartet, m=multiplet, br.=broad, etc. IR spectra were measured by a Shimazu infrared spectrometer (IR-470). Optical rotations were measured using a JASCO DIP-370 Digital Polarimeter (Japan Spectroscopic CO, Ltd.).

Chemical symbols have their usual meanings; b.p. (boiling point), m.p. (melting point), l (liter(s)), ml (milliliter(s)), g (gram(s)), mg (milligram(s)), mol (moles), mmol (millimoles), eq. (equivalent(s)).

### Example 1

### 1-[2-(3-Fluoro-4-hydroxyphenyl)-2-hydroxyethyl]-4-(6-methoxypyridin-3-yl)piperidin-4-ol methanesulfonate

1-A: 1-[4-(Benzyloxy)-3-fluorophenyl]-2-chloroethanone

To a stirred solution of 2-chloro-1-(3-fluoro-4-hydroxyphenyl)ethanone (J. Am. Chem. Soc., 1937, 59, 280) (3.00 g, 15.9 mmol) and potassium carbonate (4.40 g, 31.8 mmol) in acetone (100 mL) was added benzyl bromide (1.91 mL, 16.1 mmol) at room temperature, and the mixture was stirred at room temperature overnight. After all solvents were removed, the residue was diluted with ethyl acetate. The mixture was washed with H₂O, dried and evaporated. The residue was purified by chromatography on silica gel, eluting with methyl ethyl acetate/hexane (1:5 v/v), to afford the titled compound as a yellow solid (750 mg, 17%).

¹H NMR (270 MHz, CDCl₃) δ=7.77-7.64 (m, 2H), 7.46-7.34 (m, 5H), 7.10-7.03 (m, 1H), 5.23 (s, 2H), 4.61 (s, 2H) ppm.

MS (EI); M⁺=278

1-B: tert-Butyl 4-hydroxy-4-(6-methoxypyridin-3-yl)piperidine-1-carboxylate

A solution of 5-bromo-2-methoxypyridine (36 g, 193 mmol) in diethyl ether (200 mL) was added dropwise to a solution of n-butyl lithium in hexane (1.59M, 121 mL) and diethyl ether (500 mL) at −78° C. After addition was completed, the mixture was stirred at −78° C. for 30 minutes and to the mixture was added a solution of tert-butyl 4-oxopiperidine-1-carboxylate in diethyl ether (300 mL) at −78° C. The mixture was allowed to warm to room temperature and stirred overnight. To the mixture was added water (400 mL) and the organic layer was extracted with diethyl ether (500 mL). The combined organic layer was washed with brine and dried over NaSO4 and concentrated. The residue was purified by column chromatography on silica gel, eluting with methyl ethyl acetate/hexane (1:2 v/v), to afford the titled compound (16.5 g, 42%) as oil.

1-C: 4-(6-Methoxypyridin-3-yl)piperidin-4-ol dihydrochloride

To a stirred solution of tert-butyl 4-hydroxy-4-(6-methoxypyridin-3-yl)piperidine-1-carboxylate (15 g, 49 mmol) in ethyl acetate (300 mL) was added 4N hydrochloride in ethyl acetate (45 mL, 150 mmol) and the resulting suspension was stirred at 50° C. for 2 hours. To the suspension was added additional 4N hydrochloride in ethyl acetate (27.5 mL, 75 mmol) and stirred at 50° C. for 3 h. After cooling, the precipitate was collected and dried in vacuo for 1 hour to afford the titled compound as a white solid (12.7 g, 93%).

¹H NMR (300 MHz, DMSO-d₆) δ=9.25-8.90 (br, 2H), 8.24 (dd, J=0.5, 2.6 Hz, 1H), 7.79 (dd, J=2.6, 8.6 Hz, 1H), 6.87 (d, J=8.6 Hz, 1H), 6.00-5.40 (br, 1H), 3.86 (s, 3H), 3.30-3.00 (m, 4H), 2.30-2.10 (m, 2H), 1.86-1.76 (m, 2H) ppm.

1-D: 1-[4-(Benzyloxy)-3-fluorophenyl]-2-[4-hydroxy-4-(6-methoxypyridin-3-yl)-piperidin-1-yl]ethanone

To a stirred solution of 1-[4-(benzyloxy)-3-fluorophenyl]-2-chloroethanone (750 mg, 2.69 mmol) in ethanol (20 mL) were added 4-(6-methoxypyridin-3-yl)piperidin-4-ol dihydrochloride (908 mg, 3.23 mmol) at room temperature under nitrogen and the mixture was stirred under reflux for 5 hours. After all solvents were removed, the residue was diluted with ethyl acetate. The mixture was washed with H₂O and the organic layer was dried and evaporated to afford the titled compound as a yellow solid (1.16 g, 96%).

¹H NMR (270 MHz, DMSO-d₆) δ=8.24 (d, J=2.5 Hz, 1H), 7.92-7.82 (m, 2H), 7.77 (dd, J=8.6, 2.5 Hz, 1H), 7.52-7.32 (m, 6H), 6.75 (d, J=8.6 Hz, 1H), 5.30 (s, 2H), 4.90 (s, 1H), 3.82 (s, 3H), 3.78 (s, 2H), 2.71-2.48 (m, 4H), 1.98-1.84 (m, 2H), 1.68-1.56 (m, 2H) ppm.

MS (ESI); (M+H)⁺=451.17, (M−H)⁻=449.24

1-E: 1-{2-[4-(Benzyloxy)-3-fluorophenyl]-2-hydroxyethyl}-4-(6-methoxypyridin-3-yl)piperidin-4-ol

To a stirred solution of sodium borohydride (146 mg, 3.86 mmol) in ethanol (45 mL) was added suspension of 1-[4-(benzyloxy)-3-fluorophenyl]-2-[4-hydroxy-4-(6-methoxypyridin-3-yl)piperidin-1-yl]ethanone (1.16 g, 2.57 mmol) in ethanol (5 mL) at 0° C. and the mixture was stirred at room temperature for 2.5 hours. After all solvents were removed, the residue was diluted with dichloromethane. The mixture was washed with H₂O, dried and evaporated. The residue was purified by chromatography on silica gel, eluting with methyl alcohol/dichloromethane (1:20 v/v), to afford the titled compound as a yellow solid (648 mg, 53%).

¹H NMR (270 MHz, DMSO-d₆) δ=8.23 (d, J=2.5 Hz, 1H), 7.77 (dd, J=8.7, 2.5 Hz, 1H), 7.50-7.06 (m, 8H), 6.76 (d, J=8.7 Hz, 1H), 5.16 (s, 2H), 4.87 (s, 1H), 4.70-4.63 (m, 1H), 3.83 (s, 3H), 2.80-2.38 (m, 6H), 2.00-1.84 (m, 2H), 1.66-1.54 (m, 2H) ppm.

MS (ESI); (M+H)⁺=453.19

1-F: 1-[2-(3-Fluoro-4-hydroxyphenyl)-2-hydroxyethyl]-4-(6-methoxypyridin-3-yl)-piperidin-4-ol

A mixture of 1-{2-[4-(benzyloxy)-3-fluorophenyl]-2-hydroxyethyl}-4-(6-methoxypyridin-3-yl)piperidin-4-ol (640 mg, 1.41 mmol) and palladium, 10 wt % on activated carbon (300 mg) in methanol (20 mL) and acetic acid (5 mL) was stirred under H₂ atmosphere for 26 hours. The mixture was filtered through Celite, and the filtrate was concentrated. The residue was purified by chromatography on silica gel, eluting with methyl alcohol/dichloromethane (1:15 v/v), to afford the titled compound as a white solid (350 mg, 68%).

¹H NMR (270 MHz, DMSO-d₆) δ=9.63 (s, 1H), 8.23 (d, J=2.5 Hz, 1H), 7.77 (dd, J=8.7, 2.5 Hz, 1H), 7.09 (d, J=12.2 Hz, 1H), 7.14-6.82 (m, 2H), 6.75 (d, J=8.7 Hz, 1H), 4.85 (br.s, 2H), 4.65-4.54 (m, 1H), 3.82 (s, 3H), 2.71-1.55 (m, 10H) ppm.

1-G: 1-[2-(3-Fluoro-4-hydroxyphenyl)-2-hydroxyethyl]-4-(6-methoxypyridin-3-yl)-piperidin-4-ol methanesulfonate

Methanesulfonic acid (26.1 μL, 0.389 mmol) was added to a solution of 1-[2-(3-fluoro-4-hydroxyphenyl)-2-hydroxyethyl]-4-(6-methoxypyridin-3-yl)piperidin-4-ol (141 mg, 0.389 mmol) in methyl alcohol (3 mL). The mixture was stirred for 30 minutes at room temperature and filtered. The filtrate was evaporated and the residue was crystallized from ethanol-diisopropylether to afford the titled compound as a white amorphous (81 mg, 45%).

¹H NMR (270 MHz, DMSO-d₆) δ=9.90 (s, 1H), 9.26 (s, 1H), 8.25 (d, J=2.3 Hz, 1H), 7.77 (dd, J=8.7, 2.3 Hz, 1H), 7.26-6.80 (m, 4H), 5.10-5.00 (m, 1H), 3.85 (s, 3H), 3.82-3.16 (m, 8H), 2.34 (s, 3H), 2.42-2.09 (m, 21), 1.97-1.74 (m, 2H) ppm

MS (ESI); (N+H)⁺=363.11, (M−H)⁻=361.16

IR (KBr); 3359, 1670 cm⁻¹

### Example 2

### 4-(3,4-Dihydro-1H-isochromen-7-yl)-1-[2-(3-fluoro-4-hydroxyphenyl)-2-hydroxyethyl]piperidin-4-ol methanesulfonate

2-A: Ethyl 4-(3,4-dihydro-1H-isochromen-7-yl)-4-hydroxypiperidine-1-carboxylate

To a solution of 7-bromoisochroman (WO 9305772 A1) (5.3 g, 25 mmol) in tetrahydrofuran (35 mL) was added 1.5 M solution of n-butyllithium in hexane (17 mL, 26 mmol) dropwise at −78° C. The mixture was stirred at −78° C. for 1 hour. To this mixture was added a solution of N-carbethoxy-4-piperidone (4.3 g, 25 mmol) in tetrahydrofuran (35 mL) at −78° C. The mixture was stirred at −78° C. for an additional 1 hour and warmed to room temperature. Water (30 mL) was carefully added to the mixture and the organic layer was separated. The aqueous layer was extracted with ethyl acetate (30 mL×2). The combined organic layers were washed with saturated aqueous sodium chloride solution, dried over potassium carbonate, filtered and concentrated under reduced pressure to give 7.8 g of a white powder. The powder was washed with 2-propanol to give the titled compound as a white powder (5.7 g, 75%).

¹H NMR (270 MHz, CDCl₃) δ=7.36-7.08 (m, 3H), 4.77 (s, 2H), 4.31-3.93 (m, 4H), 4.16 (q, J=7.1 Hz, 2H), 3.44-3.20 (m, 2H), 2.93-2.80 (m, 2H), 2.09-1.91 (m, 2H), 1.81-1.67 (m, 2H), 1.28 (t, J=7.1 Hz, 3H) ppm.

2-B: 4-(3,4-Dihydro-1H-isochromen-7-yl)piperidin-4-ol

To a suspension of ethyl 4-(3,4-dihydro-1H-isochromen-7-yl)-4-hydroxypiperidine-1-carboxylate (5.7 g, 19 mmol) in ethanol (6.3 mL) was added potassium hydroxide (5.3 g, 94 mmol). The mixture was stirred under reflux for 2 hours. The mixture was concentrated under reduced pressure. The residue was diluted with dichloromethane (20 mL) and the resulting suspension was washed with water (20 mL). The organic layer was separated and the aqueous layer was extracted with dichloromethane (20 mL×2). The combined organic layers were washed with brine, dried over potassium carbonate, filtered and concentrated under reduced pressure to give the titled compound as a pale yellow color solid (3.9 g, 89%).

¹H NMR (270 MHz, CDCl₃) δ=7.38-7.23 (m, 1H), 7.20-7.02 (m, 2H), 4.77 (s, 2H), 4.03-3.90 (m, 2H), 3.20-3.02 (m, 2H), 3.00-2.77 (m, 4H), 2.12-1.90 (m, 2H), 1.87-1.58 (m, 2H) ppm.

2-C: 2-[4-(3,4-Dihydro-1H-isochromen-7-yl)-4-hydroxypiperidin-1-yl]-1-(3-fluoro-4-hydroxyphenyl)ethanone

The title compound was prepared according to the procedure described in Example 1 from 2-chloro-1-(3-fluoro-4-hydroxyphenyl)ethanone and 4-(3,4-dihydro-1H-isochromen-7-yl)piperidin-4-ol: 1.26 g (100%) as yellow solid.

¹H NMR (300 MHz, DMSO-d₆) δ=7.80-7.70 (m, 2H), 7.28-6.96 (m, 4H), 4.70-4.60 (m, 3H), 3.86 (t, J=5.7 Hz, 2H), 2.80-2.46 (m, 8H), 1.98-1.82 (m, 2H), 1.60-1.50 (m, 2H) ppm.

MS (ESI); (M+H)⁺=386.10, (M−H)⁻=384.18

2-D: 2-[4-(3,4-Dihydro-1H-isochromen-7-yl)-4-hydroxypiperidin-1-yl]-1-[3-fluoro-4-[(triisopropylsilyl)oxy]phenyl}ethanone

To a stirred solution of 2-[4-(3,4-dihydro-1H-isochromen-7-yl)-4-hydroxypiperidin-1-yl]-1-(3-fluoro-4-hydroxyphenyl)ethanone (1.26 g, 2.65 mmol) and triethylamine (1.11 mL, 7.96 mmol) in tetrahydrofuran (100 mL) was added triisopropylsilyl chloride (0.624 mL, 2.92 mmol) at room temperature, and the mixture was stirred at room temperature for 2.5 hours. The mixture was treated with H₂O and extracted with ethyl acetate. The combined organic layer was dried and evaporated to afford the titled compound as a yellow solid (1.51 g quant.).

¹H NMR (300 MHz, CDCl₃) δ=7.78 (dd, J=11.3, 2.2 Hz, 1H), 7.75-7.70 (m, 1H), 7.34-7.28 (m, 1H), 7.16-7.10 (m, 2H), 6.98 (t, J=8.4 Hz, 1H), 4.77 (s, 2H), 3.97 (t, J=5.7 Hz, 2H), 3.81 (br.s, 3H), 2.94-2.58 (m, 6H), 2.32-2.18 (m, 2H), 1.80-1.68 (m, 2H), 1.40-1.20 (m, 3H), 1.11 (d, J=7.1 Hz, 18H) ppm.

MS (ESI); (M+H)⁺=542.25

2-E: 4-(3,4-Dihydro-1H-isochromen-7-yl)-1-(2-{3-fluoro-4-[(triisopropylsilyl)-oxy]phenyl}-2-hydroxyethyl)piperidin-4-ol

The title compound was prepared according to the procedure described in Example 1 from 2-[4-(3,4-dihydro-1H-isochromen-7-yl)-4-hydroxypiperidin-1-yl)-1-{3-fluoro-4-[(triisopropylsilyl)oxy]phenyl}ethanone: 907 mg (63%) as yellow solid.

¹H NMR (300 MHz, DMSO-d₄) δ=7.24 (dd, J=7.9, 1.7 Hz, 1H), 7.17 (dd, J=12.1, 1.7 Hz, 1H), 7.12 (br.s, 1H), 7.08-7.01 (m, 2H), 6.94 (t, J=8.6 Hz, 1H), 4.99 (s, 1H), 4.72-4.60 (m, 4H), 3.86 (t, J=5.5 Hz, 2H), 2.78-2.36 (m, 8H), 1.96-1.80 (m, 2H), 1.54-1.49 (m, 2H), 1.34-1.14 (m, 3H), 1.06 (d, J=7.0 Hz, 18H) ppm.

MS (ESI); (M+H)⁺=544.25

2-F: 4-(3,4-Dihydro-1H-isochromen-7-yl)-1-[2-(3-fluoro-4-hydroxyphenyl)-2-hydroxyethyl]piperidin-4-ol

A mixture of 4-(3,4-dihydro-1H-isochromen-7-yl)-1-(2-{3-fluoro-4-[(triisopropylsilyl)oxy]phenyl}-2-hydroxyethyl)piperidin-4-ol (907 mg, 1.67 mmol) and tetrabutylammonium fluoride (435 mg, 1.67 mmol) in tetrahydrofuran (15 mL) was stirred at room temperature for 1.5 hours. After all solvents were removed, the residue was purified by chromatography on silica gel, eluting with triethylamine/ethyl acetate/hexane (0.05:1:2 v/v/v), to afford the titled compound as a white solid (484 mg, 75%).

¹H NMR (270 MHz, DMSO-d₆) δ=9.61 (br.s, 1H), 7.25 (d, J=8.1 Hz, 1H), 7.14-7.01 (m, 3H), 6.99-6.84 (m, 2H), 4.70-4.58 (m, 4H), 3.89-3.82 (m, 2H), 2.76-2.35 (m, 9H), 2.00-1.80 (m, 2H), 1.60-1.48 (m, 2H) ppm.

2-G: 4-(3,4-Dihydro-1H-isochromen-7-yl)-1-[2-(3-fluoro-4-hydroxyphenyl)-2-hydroxyethyl]piperidin-4-ol methanesulfonate

By the procedures of example 1, 4-(3,4-dihydro-1H-isochromen-7-yl)-1-[2-(3-fluoro-4-hydroxyphenyl)-2-hydroxyethyl]piperidin-4-ol was converted to the title compound obtained as a white amorphous in 89% (536 mg) after crystallization from ethanol-diisopropylether.

¹H NMR (270 MHz, DMSO-d₆) δ=9.91 (s, 1H), 9.21 (s, 1H), 7.39-6.94 (m, 6H), 5.05-5.01 (m, 1H), 4.70 (s, 2H), 3.88 (t, J=5.8 Hz, 2H), 3.62-3.23 (m, 6H), 2.76 (t, J=5.8 Hz, 2H), 2.43-2.22 (m, 2H), 2.33 (s, 3H), 1.86-1.76 (m, 2H) ppm.

MS (ESI); (M+H)⁺=388.14, (M−H)⁻=386.20

IR (KBr); 3265 cm⁻¹

### Example 3

### 1-[2-(3-Fluoro-4-hydroxyphenyl)-2-hydroxyethyl]-4-(3-fluorophenyl)piperidin-4-ol methanesulfonate

3-A: 1-(3-Fluoro-4-hydroxyphenyl)-2-[4-(3-fluorophenyl)-4-hydroxypiperidin-1-yl]ethanone

The title compound was prepared according to the procedure described in Example from 2-chloro-1-(3-fluoro-4-hydroxyphenyl)ethanone and 4-(3-fluorophenyl)piperidin-4-ol (U.S. Pat. No. 4,292,321): 644 mg (70%) as yellow solid.

¹H NMR (270 MHz, DMSO-d₆) δ=7.67-6.70 (m, 7H), 3.60-2.60 (m, 6H), 2.04-1.88 (m, 2H), 1.66-1.50 (m, 2H) ppm.

MS (ESI); (M+H)⁺=348.03

3-B: 2-[4-(3-Fluorophenyl)-4-hydroxypiperidin-1-yl]-1-{3-fluoro-4-[(triisopropylsilyl)oxy]phenyl}ethanone

The title compound was prepared according to the procedure described in Example 2 from 1-(3-fluoro-4-hydroxyphenyl)-2-[4-(3-fluorophenyl)-4-hydroxypiperidin-1-yl]ethanone: 1.38 g (95%) as yellow solid.

¹H NMR (300 MHz, DMSO-d₆) δ=7.90-7.78 (m, 2H), 7.40-7.22 (m, 3H), 7.13 (t, J=8.6 Hz, 1H), 7.06-6.98 (m, 1H), 4.97 (s, 1H), 3.80 (s, 2H), 2.73-2.50 (m, 4H), 2.00-1.88 (m, 2H), 1.62-1.53 (m, 2H), 1.40-1.23 (m, 3H), 1.07 (d, J=7.1 Hz, 18H) ppm.

MS (ESI); (M+H)⁺=504.22

C. 4-(3-Fluorophenyl)-1-(2-{3-fluoro-4-[(triisopropylsilyl)oxy]phenyl}-2-hydroxyethyl)piperidin-4-ol

The title compound was prepared according to the procedure described in Example 1 from 2-[4-(3-fluorophenyl)-4-hydroxypiperidin-1-yl]-1-{3-fluoro-4-[(triisopropylsilyl)oxy]phenyl}ethanone: 1.05 g (64%) as yellow solid.

¹H NMR (300 MHz, CDCl₃) δ=7.38-7.20 (m, 3H), 7.10 (t, J=11.7, 2.0 Hz, 1H), 7.00-6.88 (m, 3H), 4.68 (dd, J=10.4, 3.5 Hz, 1H), 3.08-1.74 (m, 10H), 1.35-1.17 (m, 3H), 1.10 (d, J=6.8 Hz, 18H) ppm.

MS (ESI); (M+H)⁺=506.22

3-D: 1-[2-(3-Fluoro-4-hydroxyphenyl)-2-hydroxyethyl]-4-(3-fluorophenyl)piperidin-4-ol

The title compound was prepared according to the procedure described in Example 2 from 4-(3-fluorophenyl)-1-(2-{3-fluoro-4-[(triisopropylsilyl)oxy]phenyl}-2-hydroxyethyl)piperidin-4-ol: 420 mg (58%) as a white solid.

¹H NMR (270 MHz, DMSO-d₆) δ=9.63 (s, 1H), 7.40-7.23 (m, 3H), 7.12-6.86 (m, 4H), 4.91 (br.s, 2H), 4.65-4.54 (m, 1H), 2.76-2.46 (m, 6H), 1.97-1.88 (m, 2H), 1.60-1.50 (m, 2H) ppm.

3-E: 1-[2-(3-Fluoro-4-hydroxyphenyl)-2-hydroxyethyl]-1-(3-fluorophenyl)piperidin-4-ol methanesulfonate

By the procedures of example 1, 1-[2-(3-fluoro-4-hydroxyphenyl)-2-hydroxyethyl]-4-(3-fluorophenyl)piperidin-4-ol was converted to the title compound obtained as a white solid in 60% (320 mg) after recrystallization from ethanol-diisopropylether.

¹H NMR (270 MHz, DMSO-d₆) δ=9.23 (s, 1H), 7.50-6.92 (m, 7H), 5.10-5.00 (m, 1H), 3.80-3.19 (m, 9H), 2.41-2.20 (m, 2H), 2.35 (s, 3H), 1.92-1.66 (m, 2H) ppm.

MS (ESI); (M+H)⁺=350.08, (M−H)⁻=348.13

IR (KBr); 3429, 1622 cm⁻¹

### Example 4

### 4-(3,4-Dihydro-1H-isochromen-7-yl)-1-[2-hydroxy-2-(4-hydroxy-3-methylphenyl)ethyl]piperidin-4-ol

4-A: 1-[4-(Benzyloxy)-3-methylphenyl]-2-bromoethanone

To a stirred solution of 1-[4-(benzyloxy)-3-methylphenyl]ethanone (WO 9723216) (2.75 g, 11.4 mmol) in 1,4-dioxane (50 mL) and ethyl acetate (10 mL) was added bromine (0.587 mL, 11.4 mmol) at room temperature, and the mixture was stirred at room temperature for 15 minutes. The mixture was treated with aq. sodium thiosulfate and extracted with ethyl acetate. The combined organic layer was dried and evaporated to afford the titled compound as a yellow oil (3.86 g, quant.).

¹H NMR (270 MHz, CDCl₃) δ=7.96-7.80 (m, 2H), 7.48-7.30 (m, 5H), 6.96-6.90 (m, 1H), 5.18 (s, 2H), 4.40 (s, 2H), 2.32 (s, 3H) ppm.

MS (EI); M⁺=318, 320

4-B: 1-[4-(Benzyloxy)-3-methylphenyl]-2-[4-(3,4-dihydro-1H-isochromen-7-yl)-4-hydroxypiperidin-1-yl]ethanone

The title compound was prepared according to the procedure described in Example 1 from 1-[(1-benzyloxy)-3-methylphenyl]-2-bromoethanone and 4-(3,4-dihydro-1H-isochromen-7-yl)piperidin-4-ol: 391 mg (42%) as a yellow solid.

¹H NMR (270 MHz, DMSO-d₆) δ=7.91 (d, J=8.7 Hz, 1H), 7.84 (s, 1H), 7.52-7.30 (m, 5H), 7.25 (d, J=8.1 Hz, 1H), 7.17-7.12 (m, 2H), 7.07 (d, J=8.1 Hz, 1H), 5.25 (s, 2H), 4.80 (br.s, 1H), 4.67 (s, 2H), 3.89-3.84 (m, 6H), 2.80-2.54 (m, 4H), 2.26 (s, 3H), 2.03-1.52 (m, 4H) ppm.

MS (ESI); (M+H)⁺=472.18

4-C: 1-{2-[4-(Benzyloxy)-3-methylphenyl]-2-hydroxyethyl}-4-(3,4-dihydro-1H-isochromen-7-yl)piperidin-4-ol

The title compound was prepared according to the procedure described in Example 1 from 1-[4-(benzyloxy)-3-methylphenyl]-2-[4-(3,4-dihydro-1H-isochromen-7-yl)-4-hydroxypiperidin-1-yl]ethanone: 266 mg (68%) as a white solid.

¹H NMR (300 MHz, CDCl₃) δ=7.48-7.22 (m, 6H), 7.16-7.04 (m, 4H), 6.94 (d, J=8.4 Hz, 1H), 5.10 (s, 2H), 4.76 (br.s, 1H), 4.70 (s, 1H), 4.67 (s, 2H), 4.67-4.59 (m, 1H), 3.86 (t, J=5.9 Hz, 2H), 2.78-2.68 (m, 4H), 2.59-2.35 (m, 4H), 2.20 (s, 3H), 2.00-1.50 (m, 4H) ppm.

4-D: 4-(3,4-Dihydro-1H-isochromen-7-yl)-1-[2-hydroxy-2-(4-hydroxy-3-methylphenyl)ethyl]piperidin-4-ol

By the procedures of example 1, 1-{2-[4-(benzyloxy)-3-methylphenyl]-2-hydroxyethyl}-4-(3,4-dihydro-1H-isochromen-7-yl)piperidin-4-ol was converted to the title compound obtained as a white solid in 60% (130 mg) after recrystallization from ethanol-diisopropylether.

¹H NMR (300 MHz, DMSO-d₆) δ=9.10 (s, 1H), 7.25 (d, J=8.2 Hz, 1H), 7.13 (s, 1H), 7.06 (d, J=8.2 Hz, 1H), 7.03 (s, 1H), 6.95 (d, J=8.0 Hz, 1H), 6.70 (d, J=8.0,Hz, 1H), 4.72-4.54 (m, 5H), 3.86 (t, J=5.3 Hz, 2H), 2.80-2.34 (m, 8H), 2.10 (s, 3H), 1.98-1.50 (m, 4H) ppm.

MS (ESI); (M+H)⁺=384.14, (M−H)⁻=382.23

m.p. 178.3° C.

IR (KBr); 3298, 1612 cm⁻¹

### Example 5

### 4-(3-Fluorophenyl)-1-[2-hydroxy-2-(4-hydroxy-3-methylphenyl)ethyl]piperidin-4-ol

5-A: 1-[4-(Benzyloxy)-3-methylphenyl]-2-[4-(3-fluorophenyl)-4-hydroxypiperidin-1-yl]ethanone

The title compound was prepared according to the procedure described in Example 1 from 1-[4-(benzyloxy)-3-methylphenyl]-2-bromoethanone and 4-(3-fluorophenyl)piperidin-4-ol: 1.4 g (quant.) as an orange solid.

MS (ESI); (M+H)⁺=434.14

5-B: 1-{2-[4-(Benzyloxy)-3-methylphenyl]-2-hydroxyethyl}-4-(3-fluorophenyl)-piperidin-4-ol

The title compound was prepared according to the procedure described in Example 1 from 1-[4-(benzyloxy)-3-methylphenyl]-2-[4-(3-fluorophenyl)-4-hydroxypiperidin-1-yl]ethanone: 627 mg (45%) as a yellow solid.

¹H NMR (300 MHz, DMSO-d₆) δ=7.47-6.93 (m, 12H), 5.11 (s, 2H), 5.20-4.60 (m, 3H), 2.79-2.19 (m, 6H), 2.21 (s, 3H), 2.05-1.95 (m, 2H), 1.62-1.52 (m, 2H) ppm.

MS (ESI); (M+H)⁺=436.17

5-C: 4-(3-Fluorophenyl)-1-[2-hydroxy-2-(4-hydroxy-3-methylphenyl)ethyl]-piperidin-4-ol

By the procedures of example 1, 1-{2-[4-(benzyloxy)-3-methylphenyl]-2-hydroxyethyl}-4-(3-fluorophenyl)piperidin-4-ol was converted to the title compound obtained as a white solid in 72% (351 mg) after recrystallization from ethanol-diisopropylether.

¹H NMR (300 MHz, DMSO-d₆) δ=9.09 (s, 1H), 7.41-7.25 (m, 3H), 7.08-6.92 (m, 3H), 6.70 (d, J=8.0 Hz, 1H), 4.92 (s, 1H), 4.66 (d, J=2.7 Hz, 1H), 4.62-4.56 (m, 1H), 2.80-2.30 (m, 6H), 2.10 (s, 3H), 2.02-1.86 (m, 2H), 1.62-1.50 (m, 2H) ppm.

MS (ESI); (M+H)⁺=346.08, (M−H)⁻=344.17

### Example 6

### 1-[2-Hydroxy-2-(4-hydroxy-3-methylphenyl)ethyl]-4-(6-methoxypyridin-3-yl)-piperidin-4-ol

6-A: 1-[4-(Benzyloxy)-3-methylphenyl]-2-[4-hydroxy-4-(6-methoxypyridin-3-yl)-piperidin-1-yl]ethanone

The title compound was prepared according to the procedure described in Example 1 from 1-[4-(benzyloxy)-3-methylphenyl]-2-bromoethanone and 4-(6-methoxypyridin-3-yl)piperidin-4-ol dihydrochloride: 683 mg (45%) as a white solid.

¹H NMR (300 MHz, DMSO-d₆) δ=8.25 (s, 1H), 7.92-7.30 (m, 8H), 7.14 (d, J=8.5 Hz, 1H), 6.77 (d, J=8.5 Hz, 1H), 5.25 (s, 2H), 5.02 (s, 1H), 3.99 (s, 2H), 3.83 (s, 3H), 2.90-2.52 (m, 4H), 2.26 (s, 3H), 2.02-1.95 (m, 2H), 1.70-1.60 (m, 2H) ppm.

MS (ESI); (M+H)⁺=447.16

6-B: 1-{2-[4-(Benzyloxy)-3-methylphenyl]-2-hydroxyethyl}-4-(6-methoxypyridin-3-yl)piperidin-4-ol

The title compound was prepared according to the procedure described in Example 1 from 1-[4-(benzyloxy)-3-methylphenyl]-2-[4-hydroxy-4-(6-methoxypyridin-3-yl)piperidin-1-yl]ethanone: 368 mg (54%) as a white solid.

MS (ESI); (M+H)⁺=449.17

6-C: 1-[2-Hydroxy-2-(4-hydroxy-3-methylphenyl)ethyl]-4-(6-methoxypyridin-3-yl)-piperidin-4-ol

By the procedures of example 1, 1-{2-[4-(benzyloxy)-3-methylphenyl]-2-hydroxyethyl}-4-(3-fluorophenyl)piperidin-4-ol was converted to the title compound obtained as a white solid in 75% (221 mg) after recrystallization from 2-propanol-diisopropylether.

¹H NMR (300 MHz, DMSO-d₆) δ=9.09 (s, 1H), 8.24 (d, J=2.6 Hz, 1H), 7.77 (dd, J=8.7, 2.6 Hz, 1H), 7.02 (d, J=2.0 Hz, 1H), 6.95 (dd, J=8.2, 2.0 Hz, 1H), 6.76 (d, J=8.7 Hz, 1H), 6.69 (d, J=8.2 Hz, 1H), 4.86 (br.s, 1H), 4.66 (br.s, 1H), 4.60-4.54 (m, 1H), 3.82 (s, 3H), 2.80-2.30 (m, 6H), 2.10 (s, 3H), 2.00-1.57 (m, 4H) ppm.

MS (ESI); (M+H)⁺=359.12, (M−H)⁻=357.20

m.p. 176.0° C.

IR (KBr); 3197, 1608 cm⁻¹

### Example 7

### 1-[2-(2-Fluoro-4-hydroxyphenyl)-2-hydroxyethyl]-4-(3-fluorophenyl)piperidin-4-ol

7-A: 1-[4-(Benzyloxy)-2-fluorophenyl]-2-bromoethanone

The title compound was prepared according to the procedure described in Example 4 from 1-[4-(benzyloxy)-2-fluorophenyl]ethanone (WO 0170702): 3.70 g (quant.) as a yellow solid.

¹H NMR (300 MHz, CDCl₃) δ=7.95 (t, J=8.8 Hz, 1H), 7.45-7.35 (m, 5H), 6.92-6.70 (m, 2H), 5.13 (s, 2H), 4.47 (d, J=2.6 Hz, 2H) ppm.

MS (EI); M⁺=322, 324

7-B: 1-{2-[4-(Benzyloxy)-2-fluorophenyl]-2-hydroxyethyl}-4-(3-fluorophenyl)-piperidin-4-ol

The title compound was prepared according to the procedure described in Example 1 from 1-[4-(benzyloxy)-2-fluorophenyl]-2-bromoethanone and 4-(3-fluorophenyl)piperidin-4-ol: 599 mg (68%) as a white solid

¹H NMR (270 MHz, DMSO-d₆) δ=7.48-7.22 (m, 9H), 7.06-6.96 (m, 1H), 6.88-6.80 (m, 2H), 5.11 (s, 2H), 5.02 (br.s, 1H), 4.98-4.88 (m, 1H), 4.91 (s, 1H), 2.76-2.40 (m, 6H), 2.00-1.83 (m, 2H), 1.60-1.50 (m, 2H) ppm.

MS (ESI); (M+H)⁺=440.14

7-C: 1-[2-(2-Fluoro-4-hydroxyphenyl)-2-hydroxyethyl]-4-(3-fluorophenyl)-piperidin-4-ol

By the procedures of example 1, 1-{2-[4-(benzyloxy)-2-fluorophenyl]-2-hydroxyethyl}-4-(3-fluorophenyl)piperidin-4-ol was converted to the title compound obtained as a white solid in 53% (254 mg) after recrystallization from 2-propanol.

¹H NMR (300 MHz, DMSO-d₆) δ=9.75 (br.s, 1H), 7.40-7.24 (m, 4H), 7.06-6.98 (m, 1H), 6.60 (dd, J=8.4, 2.2 Hz, 1H), 6.49 (dd, J=12.3, 2.2 Hz, 1H), 4.95-4.88 (m, 3H), 2.78-2.65 (m, 2H), 2.60-2.30 (m, 4H), 2.00-1.85 (m, 2H), 1.62-1.50 (m, 2H) ppm.

MS (ES); (M+H)⁺=350.08, (M−H)⁻=348.14

m.p. 186.1° C.

### Example 8

### 4-(3,4-Dihydro-1H-isochromen-7-yl)-1-[2-(2-fluoro-4-hydroxyphenyl)-2-hydroxyethyl]piperidin-4-ol

8-A: 1-[4-(Benzyloxy)-3-fluorophenyl]-2-[4-(3,4-dihydro-1H-isochromen-7-yl)-4-hydroxypiperidin-1-yl]ethanone

The title compound was prepared according to the procedure described in Example 1 from 1-[4-(benzyloxy)-2-fluorophenyl]-2-bromoethanone and 4-(3,4-dihydro-1H-isochromen-7-yl)piperidin-4-ol: 792 mg (quant.) as a yellow solid

¹H NMR (270 MHz, DMSO-d₆) δ=7.54-6.94 (m, 1H), 5.22 (s, 2H), 4.72 (br.s, 1H), 4.66 (s, 2H), 3.85 (t, J=5.6 Hz, 2H), 3.68 (s, 2H), 2.73 (t, J=5.6 Hz, 2H), 2.70-2.40 (m, 4H), 1.94-1.49 (m, 4H) ppm.

MS (ESI); (M+H)⁺=476.18

8-B: 1-{2-[4-(Benzyloxy)-3-fluorophenyl]-2-hydroxyethyl}-4-(3,4-dihydro-1H-isochromen-7-yl)piperidin-ol

The title compound was prepared according to the procedure described in Example 1 from 1-[4-(benzyloxy)-3-fluorophenyl]-2-[4-(3,4-dihydro-1H-isochromen-7-yl)-4-hydroxypiperidin-1-yl]ethanone: 514 mg (66%) as a yellow solid

¹H NMR (270 MHz, DMSO-d₆) δ=7.48-7.30 (m, 6H), 7.24 (dd, J=7.9, 1.2 Hz, 1H), 7.12 (br.s, 1H), 7.06 (d, J=7.9 Hz, 1H), 6.88-6.80 (m, 2H), 5.11 (s, 2H), 5.01 (br.s, 1H), 4.93 (br.s, 1H), 4.68 (s, 1H), 4.66 (s, 2H), 3.86 (t, J=5.6 Hz, 2H), 2.73 (t, J=5.6 Hz, 2H), 2.70-2.39 (m, 6H), 1.96-1.81 (m, 2H), 1.60-1.48 (m, 2H) ppm.

MS (ESI); (M+H)⁺=478.20

8-C: 4-(3,4-Dihydro-1H-isochromen-7-yl)-1-[2-(3-fluoro-4-hydroxyphenyl)-2-hydroxyethyl]piperidin-4-ol

By the procedures of example 1, 1-{2-[4-(benzyloxy)-3-fluorophenyl]-2-hydroxyethyl}-4-(3,4-dihydro-1H-isochromen-7-yl)piperidin-4-ol was converted to the title compound obtained as a white solid in 45% (190 mg) after recrystallization from 2-propanol.

¹H NMR (270 MHz, DMSO-d₆) δ=9.71 (br.s, 1H), 7.31-7.20 (m, 2H), 7.12 (s, 1H), 7.06 (d, J=8.0 Hz, 1H), 6.59 (dd, J=8.4, 2.3 Hz, 1H), 6.48 (dd, J=12.3, 2.3 Hz, 1H), 4.95-4.85 (m, 1H), 4.70 (s, 1H), 4.66 (s, 2H), 3.86 (t, J=5.8 Hz, 2H), 2.76-2.68 (m, 4H), 2.59-2.43 (m, 4H), 1.92-1.49 (m, 4H) ppm.

MS (ESI); (M+H)⁺=388.11, (M−H)⁻=386.13

m.p. 186.1° C.

### Example 9

### 1-[2-(2-Fluoro-4-hydroxyphenyl)-2-hydroxyethyl]-4-(6-methoxypyridin-3-yl)piperidin-4-ol

9-A: 1-{2-[4-(Benzyloxy)-2-fluorophenyl]-2-hydroxyethyl}-4-(6-methoxypyridin-3-yl)piperidin-4-ol

The title compound was prepared according to the procedure described in Example 1 from 1-[4-(benzyloxy)-2-fluorophenyl]-2-bromoethanone and 4-(6-methoxypyridin-3-yl)piperidin-4-ol dihydrochloride: 386 mg (43%) as a yellow solid

¹H NMR (270 MHz, DMSO-d₆) δ=8.23 (d, J=2.3 Hz, 1H), 7.80-7.73 (m, 1H), 7.50-7.34 (m, 6H), 6.90-6.80 (m, 2H), 6.75 (d, J=8.6 Hz, 1H), 5.11 (s, 2H), 4.98-4.92 (m, 1H), 4.86 (s, 1H), 3.82 (s, 3H), 2.77-2.40 (m, 6H), 1.97-1.55 (m, 4H) ppm.

MS (ESI); (M+H)⁺=453.19

9-B: 1-[2-(2-Fluoro-4-hydroxyphenyl)-2-hydroxyethyl]-4-(6-methoxypyridin-3-yl)piperidin-4-ol

By the procedures of example 1, 1-{2-[4-(benzyloxy)-2-fluorophenyl]-2-hydroxyethyl}-4-(6-methoxypyridin-3-yl)piperidin-4-ol was converted to the title compound obtained as a white amorphous in 39% (120 mg) after crystallization from 2-propanol-diisopropylether.

¹H NMR (270 MHz, DMSO-d₆) δ=9.74 (s, 1H), 8.22 (br.s, 1H), 7.76 (dd, J=8.7, 1.8 Hz, 1H), 7.28 (t, J=8.6 Hz, 1H), 6.76 (d, J=8.7 Hz, 1H), 6.60 (d, J=8.6 Hz, 1H), 6.49 (d, J=12.0 Hz, 1H), 4.92 (br.s, 3H), 3.82 (s, 3H), 2.77-2.24 (m, 6H), 2.02-1.85 (m, 2H), 1.70-1.56 (m, 2H) ppm.

MS (ESI); (M+H)⁺=363.08, (M−H)⁻=361.13

### Example 10

### 4-(3-Fluorophenyl)-1-[2-hydroxy-2-(4-hydroxyphenyl)ethyl]piperidin-4-ol

10-A: 1-[4-(Benzyloxy)phenyl]-2-[4-(3-fluorophenyl)-4-hydroxypiperidin-1-yl]ethanone

The title compound was prepared according to the procedure described in Example 1 from 1-[4-(benzyloxy)phenyl]-2-bromoethanone (Indian J. Chem. Sect. B, 1979, 17, 305) and 4-(3-fluorophenyl)piperidin-4-ol: 982 mg (quant.) as a brown solid

¹H NMR (300 MHz, DMSO-d₆) δ=8.02 (d, J=8.8 Hz, 2H), 7.48-7.17 (m, 8H), 7.12 (d, J=8.8 Hz, 2H), 7.06-6.98 (m, 1H), 5.22 (s, 2H), 4.98 (s, 1H), 3.79 (s, 2H), 2.76-2.57 (m, 4H), 2.00-1.86 (m, 2H), 1.60-1.54 (m, 2H) ppm.

MS (ESI); (M+H)⁺=420.11

10-B: 1-{2-[4-(Benzyloxy)phenyl]-2-hydroxyethyl}-4-(3-fluorophenyl)piperidin-4-ol

The title compound was prepared according to the procedure described in Example 1 from 1-[4-(benzyloxy)phenyl]-2-[4-(3-fluorophenyl)-4-hydroxypiperidin-1-yl]ethanone: 653 mg (77%) as a yellow solid.

¹H NMR (270 MHz, DMSO-d₆) δ=7.48-7.20 (m, 101), 7.08-6.92 (m, 3H), 5.09 (s, 2H), 4.91 (s, 1H), 4.82 (s, 1H), 4.70-4.62 (m, 1H), 2.75-2.36 (m, 6H), 1.96-1.87 (m, 2H), 1.58-1.53 (m, 2H) ppm.

MS (ESI); (M+H)⁺=422.15

10-C: 4-(3-Fluorophenyl)-1-[2-hydroxy-2-(4-hydroxyphenyl)ethyl]piperidin-4-ol

By the procedures of example 1, 1-{2-[4-(benzyloxy)phenyl]-2-hydroxyethyl}-4-(3-fluorophenyl)piperidin-4-ol was converted to the title compound obtained as a white solid in 49% (251 mg) after recrystallization from 2-propanol.

¹H NMR (300 MHz, DMSO-d₆) δ=9.22 (s, 1H), 7.40-7.24 (m, 3H), 7.14 (d, J=8.4 Hz, 2H), 7.06-6.88 (m, 1H), 6.70 (d, J=8.4 Hz, 2H), 4.92 (s, 1H), 4.72 (s, 1H), 4.61 (br.s, 1H), 2.80-2.34 (m, 6H), 2.02-1.85 (m, 2H), 1.60-1.50 (m, 2H) ppm.

MS (ESI); (M+H)⁺=332.07, (M−H)⁻=330.17

m.p. 154.2° C.

IR (KBr); 3325, 1616 cm⁻¹

### Example 11

### 1-[2-hydroxy-2-(4-hydroxyphenyl)ethyl]-4-(6-methoxypyridin-3-yl)piperidin-4-ol

11-A: 1-[4-(Benzyloxy)phenyl]-2-[4-hydroxy-4-(6-methoxypyridin-3-yl)piperidin-1-yl]ethanone

The title compound was prepared according to the procedure described in Example 1 from 1-[4-(benzyloxy)phenyl]-2-bromoethanone and 4-(6-methoxypyridin-3-yl)piperidin-4-ol dihydrochloride: 789 mg (91%) as a brown solid

¹H NMR (300 MHz, DMSO-d₆) δ=8.24 (d, J=2.4 Hz, 1H), 8.01 (d, J=8.4 Hz, 2H), 7.77 (dd, J=8.6, 2.4 Hz, 1H), 7.49-7.32 (m, 5H), 7.12 (d, J=8.4 Hz, 2H), 6.75 (d, J=8.6 Hz, 1H), 5.21 (br.s, 3H), 3.82 (s, 3H), 3.77 (s, 2H), 2.72-2.50 (m, 4H), 1.98-1.86 (m, 2H), 1.63-1.59 (m, 2H) ppm.

MS (ESI); (M+H)⁺=433.16

11-B: 1-{2-[4-(Benzyloxy)phenyl]-2-hydroxyethyl}-4-(6-methoxypyridin-3-yl)-piperidin-4-ol

The title compound was prepared according to the procedure described in Example 1 from 1-[4-(benzyloxy)phenyl]-2-[4-hydroxy-4-(6-methoxypyridin-3-yl)piperidin-1-yl]ethanone: 515 mg (59%) as a yellow solid

¹H NMR (270 MHz, DMSO-d₆) δ=8.23 (d, J=2.5 Hz, 1 H), 7.77 (dd, J=8.7, 2.5 Hz, 1H), 7.48-7.30 (m, 5H), 7.27 (d, J=8.6 Hz, 2H), 6.95 (d, J=8.6 Hz, 2H), 6.75 (d, J=8.7 Hz, 1H), 5.09 (br.s, 2H), 4.86 (s, 2H), 4.70-4.63 (m, 1H), 3.83 (s, 3m), 2.74-2.36 (m, 6H), 2.02-1.82 (m, 2H), 1.66-1.56 (m, 2H) ppm.

MS (ESI); (M+H)⁺=435.16

11-C: 1-{2-[4-(Benzyloxy)phenyl]-2-hydroxyethyl}-4-(6-methoxypyridin-3-yl)-piperidin-4-ol

By the procedures of example 1, 1-{2-[4-(benzyloxy)phenyl]-2-hydroxyethyl}-4-(6-methoxypyridin-3-yl)piperidin-4-ol was converted to the title compound obtained as a white solid in 61% (246 mg) after recrystallization from 2-propanol.

¹H NMR (300 MHz, DMSO-d₆) δ=9.22 (s, 1H), 8,23 (d, J=2.2 Hz, 1H), 7.77 (dd, J=8.6, 2.2 Hz, 1H), 7.13 (d, J=8.0 Hz, 2H), 6.75 (d, J=8.6 Hz, 1H), 6.69 (d, J=8.0 Hz, 2H), 4.86 (s, 1H), 4.73 (s, 1H), 4.61 (br.s, 1H), 3.82 (s, 3H), 2.68-2.34 (m, 6H), 1.96-1.86 (m, 2H), 1.65-1.55 (m, 2H) ppm.

MS (ESI); (M+H)⁺=345.08, (M−H)⁻=343.15

m.p. 198.8° C.

IR (KBr); 3471, 3385, 1609 cm⁻¹

### Example 12

### 1-[2-Hydroxy-2-(4-hydroxyphenyl)ethyl]-4-[4-(methoxymethyl)phenyl]piperidin-4-ol

12-A: 4-[4-(Methoxymethyl)phenyl]piperidin-4-ol

To a stirred solution of 1-bromo-4-(methoxymethyl)benzene (J. Med. Chem. 1998, 41, 940) (3.4 g, 20 mmol) in tetrahydrofuran (60 mL), n-butyllithium (1.56 M in hexane, 13.5 mL, 21 mmol) was added at −78° C. and the mixture was stirred for 1 hour. Then to the mixture, a solution of ethyl 4-oxopiperidine-1-carboxylate in tetrahydrofuran was added at −78° C. and the mixture was stirred at room temperature for 16 hours. The mixture was treated with sat. aq. ammonium chloride and extracted with dichloromethane. The extract was dried and evaporated. The residue was dissolved with ethanol (10 mL). To the solution, potassium hydroxide (5.6 g, 100 mmol) was added and the mixture was refluxed for 2 hours. The mixture was diluted with water and extracted with dichloromethane. The combined organic layer was dried and evaporated. The residue was purified by chromatography on amine-silica gel, eluting with methyl alcohol/dichloromethane (1:8 v/v), to afford the titled compound as a yellow oil (450 mg).

¹H NMR (270 MHz, CDCl₃) δ=7.50 (d, J=8.1 Hz, 2H), 7.34 (d, J=8.1 Hz, 2H), 4.45 (s, 2H), 3.40 (s, 3H), 3.19-2.97 (m, 4H), 2.10-1.96 (m, 2H), 1.78-1.70 (m, 2H) ppm.

12-B: 1-[4-(Benzyloxy)phenyl]-2-{4-hydroxy-4-[4-(methoxymethyl)phenyl]piperidin-1-yl}ethanone

The title compound was prepared according to the procedure described in Example 1 from 1-[4-(benzyloxy)phenyl]-2-bromoethanone and 4-[4-(methoxymethyl)phenyl]piperidin-4-ol: 954 mg (quant.) as a yellow solid.

¹H NMR (300 MHz, DMSO-d₆) δ=8.02 (d, J=8.8 Hz, 2H), 7.50-7.30 (m, 7H), 7.25 (d, J=8.1 Hz, 2H), 7.13 (d, J=8.8 Hz, 2H), 5.20 (s, 2H), 4.84 (s, 1H), 4.37 (s, 2H), 3.81 (s, 2H), 3.26 (s, 3H), 3.03-2.59 (m, 4H), 2.02-1.88 (m, 2H), 1.61-1.56 (m, 2H) ppm.

MS (ESI); (M+H)⁺=446.18

12-C: 1-{2-[4-(Benzyloxy)phenyl]-2-hydroxyethyl}-4-[4-(methoxymethyl)phenyl]piperidin-4-ol

The title compound was prepared according to the procedure described in Example 1 from 1-[4-(benzyloxy)phenyl]-2-{4-hydroxy-4-[4-(methoxymethyl)phenyl]piperidin-1-yl}ethanone: 597 mg (67%) as a yellow solid.

¹H NMR (300 MHz, DMSO-d₆) δ=7.48-7.22 (m, 1H), 6.96 (d, J=8.6 Hz, 2H), 5.09 (s, 2H), 4.84 (s, 1H), 4.77 (s, 1H), 4.68-4.66 (m, 1H), 4.37 (s, 2H), 3.27 (s, 3H), 2.78-2.36 (m, 6H), 2.02-1.85 (m, 2H), 1.62-1.50 (m, 2H) ppm.

MS (ESI); (M+H)⁺=448.18

12-D: 1-[2-Hydroxy-2-(4-hydroxyphenyl)ethyl]-4-[4-(methoxymethyl)phenyl]-piperidin-4-ol

By the procedures of example 1, 1-{2-[4-(benzyloxy)phenyl]-2-hydroxyethyl}-4-(methoxymethyl)phenyl]piperidin-4-ol was converted to the title compound obtained as a white solid in 65% (307 mg) after recrystallization from 2-propanol.

¹H NMR (270 MHz, DMSO-d₆) δ=9.22 (s, 1H), 7.45 (d, J=8.2 Hz, 2H), 7.25 (d, J=8.2 Hz, 2H), 7.14 (d, J=8.3 Hz, 2H), 6.70 (d, J=8.3 Hz, 2H), 4.77 (br.s, 2H), 4.65-4.58 (m, 1H), 4.37 (s, 2H), 3.27 (s, 3H), 2.72-2.37 (m, 6H), 2.00-1.86 (m, 2H), 1.62-1.52 (m, 2H) ppm.

MS (ESI); (M+H)⁺=358.10, (M−H)⁻=356.20

m.p. 169.6° C.

IR (KBr); 3441, 3251, 1612 cm−1

### Example 13

### 1-[2-Hydroxy-2-(4-hydroxy-3-methylphenyl)ethyl]-4-[4-(methoxymethyl)phenyl]piperidin-4-ol

13-A: 1-{3-Methyl-4-[(triisopropylsilyl)oxy]phenyl}ethanone

The title compound was prepared according to the procedure described in Example 2 from 1-(4-hydroxy-3-methylphenyl)ethanone: 9.64 g (94%) as a colorless oil.

¹H NMR (270 MHz, CDCl₃) δ=7.78 (d, J=2.3 Hz, 1H), 7.70 (d, J=8.4, 2.3 Hz, 1H), 6.80 (d, J=8.4 Hz, 1H), 2.54 (s, 3H), 2.27 (s, 3H), 1.39-1.25 (m, 3H), 1.12 (d, J=7.3 Hz, 18H) ppm.

MS (EI); M⁺=306

13-B: 2-Bromo-1-{3-methyl-4-[(triisopropylsilyl)oxy]phenyl}ethanone

The title compound was prepared according to the procedure described in Example 4 from 1-{3-methyl-4-[(triisopropylsilyl)oxy]phenyl}ethanone: 4.43 g (71%) as a colorless oil.

¹H NMR (300 MHz, CDCl₃) δ=7.81 (d, J=2.4 Hz, 1H), 7.74 (dd, J=8.6, 2.4 Hz, 1H), 6.82 (d, J=8.6 Hz, 1H), 4.01 (s, 21H), 2.28 (s, 3H), 1.38-1.26 (m, 3H), 1.12 (d, J=7.1 Hz, 18H) ppm.

MS (EI); M⁺=384, 386

13-C: 2-{4-Hydroxy-4-[4-(methoxymethyl)phenyl]piperidin-1-yl}-1-{3-methyl-4-[(triisopropylsilyl)oxy]phenyl}ethanone

The title compound was prepared according to the procedure described in Example 1 from 2-bromo-1-{3-methyl-4-[(triisopropylsilyl)oxy]phenyl}ethanone and 4-[4-(methoxymethyl)phenyl]piperidin-4-ol: 1.13 g (quant.) as a yellow solid.

¹H NMR (270 MHz, DMSO-d₆) δ=7.87-7.80 (m, 2H), 7.45 (d, J=8.0 Hz, 2H), 7.25 (d, J=8.0 Hz, 2H), 6.88 (d, J=8.4 Hz, 1H), 4.83 (s, 1H), 4.38 (s, 2H), 3.81 (s, 2H), 3.27 (s, 3H), 2.71-2.55 (m, 4H), 2.24 (s, 3H), 2.02-1.90 (m, 2H), 1.65-1.55 (m, 2H), 1.42-1.12 (m, 3H), 1.09 (d, J=7.3 Hz, 18H) ppm.

MS (ESI); (M+H)⁺=526.28

13-D: 1-(2-Hydroxy-2-{3-methyl-4-[(triisopropylsilyl)oxy]phenyl}ethyl)-4-[4-(methoxymethyl)phenyl]piperidin-4-ol

The title compound was prepared according to the procedure described in Example 1 from 2-{4-hydroxy-4-[4-(methoxymethyl)phenyl]piperidin-1-yl}-1-{3-methyl-4-[(triisopropylsilyl)oxy]phenyl}ethanone: 1.1 g (quant.) as a yellow oil.

¹H NMR (270 MHz, DMSO-d₆) δ=7.45 (d, J=8.3 Hz, 2H), 7.25 (d, J=8.3 Hz, 2H), 7.12 (s, 1H), 7.05 (d, J=8.4 Hz, 1H), 6.72 (d, J=8.4 Hz, 1H), 4.76 (s, 1H), 4.62 (br.s, 1H), 4.37 (br.s, 3H), 3.27 (s, 3H), 2.69-2.41 (m, 6H), 2.18 (s, 3H), 1.97-1.50 (m, 4H), 1.34-1.23 (m, 3H), 1.07 (d, J=7.1 Hz, 18H) ppm.

MS (ESI); (M+H)⁺=528.29

13-E: 1-[2-Hydroxy-2-(4-hydroxy-3-methylphenyl)ethyl]-4-[4-(methoxymethyl)-phenyl]piperidin-4-ol

By the procedures of example 2, 1-(2-hydroxy-2-{3-methyl-4-[(triisopropylsilyl)oxy]phenyl}ethyl)-4-[4-(methoxymethyl)phenyl]piperidin-4-ol was converted to the title compound obtained as a white solid in 69% (510 mg) after recrystallization from 2-propanol.

¹H NMR (270 MHz, DMSO-d₆) δ=9.09 (s, 1H), 7.46 (d, J=8.1 Hz, 2H), 7.25 (d, J=8.1 Hz, 2H), 7.03 (s, 1H), 6.95 (d, J=8.1 Hz, 1H), 6.70 (d, J=8.1 Hz, 1H), 4.76 (s, 1H), 4.66 (s, 1H), 4.58 (br.s, 1H), 4.37 (s, 2H), 3.27 (s, 3H), 2.72-2.32 (m, 6H), 2.10 (s, 3H), 2.04-1.85 (m, 2H), 1.62-1.52 (m, 2H) ppm.

MS (ESI); (M+H)⁺=372.12, (M−H)⁻=370.19

m.p. 164.4° C.

IR (KBr); 3472, 3163, 1611 cm⁻¹

### Example 14

### 1-[2-Hydroxy-2-(4-hydroxy-3-methylphenyl)ethyl]-4-(5-methyl-1,3-thiazol-2-yl)piperidin-4-ol

14-A: Ethyl 4-(5-Methyl-1,3-thiazol-2-yl)-4-hydroxypiperidine-1-carboxylate

The title compound is prepared from 5-methylthiazole (14.2 g) inseared of 7-bromoisochroman according to the method described in Example 2 part A as oil (13.8 g).

¹H NMR (270 MHz, CDCl3) δ=7.33 (d, J=1.2 Hz, 1H), 4.15 (d, J=7.1 Hz, 2H), 4.10-3.98 (m, 2H), 3.33 (t, J=11.3 Hz, 2H), 2.46 (d, J=1.2, 3H), 2.05 (dt, J=4.8, 13.7 Hz, 2H), 1.90-1.80 (m, 2H), 1.27 (t, J=7.1, 3H) ppm.

14-B: 4-(5-Methyl-1,3-thiazol-2-yl)piperidine-4-ol

The title compound is prepared from Ethyl 4-(5-Methyl-1,3-thiazol-2-yl)-4-hydroxypiperidine-1-carboxylate (1.0 g) inseared of ethyl 4-(3,4-dihydro-1H-isochromen-7-yl)-4-hydroxypiperidine-1-carboxylate according to the method described in Example 2 part B as oil (0.67 g).

¹H NMR (300 MHz, CDCl3) δ=7.34 (q, J=1 Hz, 1H), 3.15-2.94 (m, 4H), 2.45 (d, J=1 Hz, 3H), 2.12-2.01 (m, 2H), 1.95 (br, 1H), 1.89-1.80 (m, 2H) ppm.

14-C: 2-[4-Hydroxy-4-(5-methyl-1,3-thiazol-2-yl)piperidin-1-yl]-1-{3-methyl-4-[(triisopropylsilyl)oxy]phenyl}ethanone

The title compound was prepared according to the procedure described in Example 1 from 2-bromo-1-{3-methyl-4-[(triisopropylsilyl)oxy]phenyl}ethanone and 4-(5-methyl-1,3-thiazol-2-yl)piperidin-4-ol: 1.12 g (quant.) as a yellow oil.

¹H NMR (270 MHz, DMSO-d₆) δ=7.84-7.81 (m, 2H), 7.35 (s, 1H), 6.87 (d, J=7.1 Hz, 1H), 5.77 (s, 1H), 3.75 (s, 2H), 2.76-2.40 (m, 4H), 2.39 (s, 3H), 2.23 (s, 3H), 2.16-1.99 (m, 2H), 1.72-1.62 (m, 2H), 1.40-1.14 (m, 3H), 1.08 (d, J=7.4 Hz, 18H) ppm.

MS (ESI); (M+H)⁺=503.22

14-D: 1-(2-Hydroxy-2-{3-methyl-4-[(triisopropylsilyl)oxy]phenyl}ethyl)-4-(5-methyl-1,3-thiazol-2-yl)piperidin-4-ol

The title compound was prepared according to the procedure described in Example 1 from 2-2-[4-hydroxy-4-(5-methyl-1,3-thiazol-2-yl)piperidin-1-yl]-1-{3-methyl-4-[(triisopropylsilyl)oxy]phenyl}ethanone: 1.01 g (quant.) as a yellow oil.

¹H NMR (300 MHz, DMSO-d₆) δ=7.35 (d, J=1.3 Hz, 1H), 7.11 (d, J=2.4 Hz, 1H), 7.03 (dd, J=8.2, 2.4 Hz, 1H), 6.71 (d, J=8.2 Hz, 1H), 5.73 (s, 1H), 4.78 (s, 1H), 4.59 (br.s, 1H), 2.80-2.02 (m, 8H), 2.39 (d, J=1.3 Hz, 3H), 2.17 (s, 3H), 1.67-1.62 (m, 2H), 1.34-1.21 (m, 3H), 1.07 (d, J=7.3 Hz, 18H) ppm.

MS (ESI); (M+H)⁺=505.22

14-E: 1-[2-Hydroxy-2-(4-hydroxy-3-methylphenyl)ethyl]-4-(5-methyl-1,3-thiazol-2-yl)piperidin-4-ol

By the procedures of example 2, 1-(2-hydroxy-2-{3-methyl-4-[(triisopropylsilyl)oxy]phenyl}ethyl)-4-(5-methyl-1,3-thiazol-2-yl)piperidin-4-ol was converted to the title compound obtained as a white solid in 57% (394 mg) after recrystallization from 2-propanol.

¹H NMR (270 MHz, DMSO-d₆) δ=8.97 (s, 1H), 7.23 (s, 1H), 6.90 (s, 1H), 6.82 (d, J=8.1 Hz, 1H), 6.57 (d, J=8.1 Hz, 1H), 5.61 (s, 1H), 4.54 (s, 1H), 4.48-4.40 (m, 1H), 2.64-2.16 (m, 6H), 2.27 (s, 3H), 2.00-1.84 (m, 2H), 1.98 (s, 3H), 1.58-1.49 (m, 2H) ppm.

MS (ESI); (M+H)⁺=349.05

m.p. 163.7° C.

IR (KBr); 3254, 1612 cm⁻¹

### Example 15

### 1-[2-Hydroxy-2-(4-hydroxy-3-methylphenyl)ethyl]-4-(3-methylphenyl)piperidin-4-ol hydrochloride

15-A: 2-[4-Hydroxy-4-(3-methoxyphenyl)piperidin-1-yl]-1-{3-methyl-4-[(triisopropylsilyl)oxy]phenyl}ethanone

The title compound was prepared according to the procedure described in Example 1 from 2-bromo-1-{3-methyl-[(triisopropylsilyl)oxy]phenyl}ethanone and 4 4-(3-methoxyphenyl)piperidin-4-ol (U.S. Pat. No. 5,668,151): 1.06 g (quant.) as a yellow oil.

¹H NMR (300 MHz, DMSO-d₆) δ=7.85-7.75 (m, 2H), 7.25-7.17 (m, 1H), 7.05-7.01 (m, 2H), 6.88 (d, J=8.3 Hz, 1H), 6.79-6.75 (m, 1H), 4.81 (s, 1H), 3.77-3.72 (m, 5H), 2.73-2.55 (m, 4H), 2.24 (s, 3H), 1.98-1.89 (m, 2H), 1.59-1.54 (m, 2H), 1.40-1.21 (m, 3H), 1.08 (d, J=7.3 Hz, 18H) ppm.

MS (ESI); (M+H)⁺=512.28

15-B: 1-(2-Hydroxy-2-{3-methyl-4-[(triisopropylsilyl)oxy]phenyl}ethyl)-4-(3-methoxyphenyl)piperidin-4-ol

The title compound was prepared according to the procedure described in Example 1 from 2-[4-hydroxy-4-(3-methoxyphenyl)piperidin-1-yl]-1-{3-methyl-4-[(triisopropylsilyl)oxy]phenyl}ethanone): 1.01 g (quant.) as a yellow solid.

¹H NMR (270 MHz, DMSO-d₆) δ=7.26-6.70 (m, 7H), 4.76 (s, 1H), 4.64-4.58 (m, 1H), 3.75 (s, 3H), 2.73-2.34 (m, 6H), 2.18 (s, 3H), 1.99-1.87 (m, 2H), 1.57-1.51 (m, 2H), 1.35-1.23 (m, 3H), 1.07 (d, J=7.1 Hz, 18H) ppm.

MS (ESI); (M+H)⁺=514.30

15-C: 1-[2-Hydroxy-2-(4-hydroxy-3-methylphenyl)ethyl]-4-(3-methoxyphenyl)-piperidin-4-ol

The title compound was prepared according to the procedure described in Example 2 from 1-(2-hydroxy-2-{3-methyl-4-[(triisopropylsilyl)oxy]phenyl}ethyl)-4-(3-methoxyphenyl)piperidin-4-ol: 433 mg (61%) as a white solid.

¹H NMR (270 MHz, DMSO-d₆) δ=9.08 (s, 1H), 7.22 (t, J=8.1 Hz, 1H), 7.05-6.93 (m, 4H), 6.78-6.74 (m, 1H), 6.70 (t, J=8.1 Hz, 1H), 4.76 (s, 1H), 4.66 (s, 1H), 4.60-4.55 (m, 1H), 3.75 (s, 3H), 2.73-2.34 (m, 6H), 2.10 (s, 3H), 2.00-1.86 (m, 2H), 1.60-1.50 (m, 2H) ppm.

MS (ESI); (M+H)⁺=358.13, (M−H)⁻=356.17

15-D: 1-[2-Hydroxy-2-(4-hydroxy-3-methylphenyl)ethyl]-4-(3-methoxyphenyl)piperidin-4-ol hydrochloride

By the procedures of example 1, 1-[2-hydroxy-2-(4-hydroxy-3-methylphenyl)ethyl]-4-(3-methoxyphenyl)piperidin-4-ol was converted to the title compound obtained as a white amorphous in 97% (456 mg) after crystallization from 2-propanol-diisopropylether.

¹H NMR (300 MHz, DMSO-d₆) δ=9.69 (s, 1H), 9.37 (s, 1H), 7.29 (t, J=8.1 Hz, 1H), 7.12 (s, 1H), 7.08-7.02 (m, 3H), 6.84 (dd, J=8.1, 2.4 Hz, 1H), 6.78 (t, J=8.1 Hz, 1H), 6.01 (s, 1H), 5.45 (s, 1H), 4.98 (br.s, 1H), 3.76 (s, 3H), 3.64-3.20 (m, 6H), 2.49-2.33 (m, 2H), 2.13 (s, 3H), 1.84-1.70 (m, 2H) ppm.

MS (ESI); (M+H)⁺=358.16, (M−H)⁻=356.23

IR (KBr); 3226, 1610 cm⁻¹

### Example 16

### 4-(3-Fluorophenyl)-1-[2-hydroxy-2-(4-hydroxy-2,5-diethylphenyl)ethyl]-piperidin-4-ol

16-A: 2-Chloro-1-(4-hydroxy-2,5-dimethylphenyl)ethanone

To a stirred suspension of aluminium trichloride (32.5 g, 244 mmol) in carbon disulfide (200 mL) was added chloroacetyl chloride (7.26 mL, 90.0 mmol) at room temperature, and the mixture was stirred for 15 minutes. To the mixture, a solution of 2,5-dimethylphenol (10 g, 81.8 mmol) in carbon disulfide (50 mL) was added and the mixture was stirred under reflux for 10 hours. The mixture was poured onto ice-H₂O and extracted with ethyl acetate. The combined organic layer was washed with aq. sodium hydrogen carbonate, dried and evaporated. The residue was crystallized from hexane to afford the titled compound as an orange solid (14 g).

¹H NMR (270 MHz, DMSO-d₆) δ=10.20 (s, 1H), 7.70 (s, 1H), 6.69 (s, 1H), 4.98 (s, 2H), 2.37 (s, 3H), 2.13 (s, 3H) ppm.

MS (EI); M⁺=198

16-B: 2-Chloro-1-{2,5-dimethyl-4-[(triisopropylsilyl)oxy]phenyl}ethanone

The title compound was prepared according to the procedure described in Example 2 from 2-chloro-1-(4-hydroxy-2,5-dimethylphenyl)ethanone: 7.9 g (quant.) as a yellow solid.

¹H NMR (270 MHz, CDCl₃) δ=7.50 (s, 1H), 6.65 (s, 1H), 4.63 (s, 2H), 2.50 (s, 3H), 2.24 (s, 3H), 1.40-1.24 (m, 3H), 1.12 (d, J=7.1 Hz, 18H) ppm.

MS (EI); M⁺=355

16-C: 1-{2,5-Dimethyl-4-[(triisopropylsilyl)oxy]phenyl}-2-[4-(3-fluorophenyl)-4-hydroxypiperidin-1-yl]ethanone

The title compound was prepared according to the procedure described in Example 1 from 2-chloro-1-{2,5-dimethyl-1-[(triisopropylsilyl)oxy]phenyl}ethanone and 4-(3-fluorophenyl)piperidin-4-ol: 1.08 g (quant.) as a yellow solid.

¹H NMR (270 MHz, DMSO-d₆) δ=7.73 (s, 1H), 7.42-7.22 (m, 3H), 7.07-6.98 (m, 1H), 6.63 (s, 1H), 4.94 (s, 1H), 3.70 (s, 2H), 3.01-2.55 (m, 4H), 2.37 (s, 3H), 2.20 (s, 3H), 1.97-1.83 (m, 2H), 1.60-1.53 (m, 2H), 1.39-1.19 (m, 3H), 1.08 (d, J=7.2 Hz, 18H) ppm.

MS (ESI); (M+H)⁺=514.33

16-D: 1-(2-{2,5-Dimethyl-4-[(triisopropylsilyl)oxy]phenyl}-2-hydroxyethyl)-4-(3-fluorophenyl)piperidin-4-ol

The title compound was prepared according to the procedure described in Example 1 from 1-{2,5-dimethyl-4-[(triisopropylsilyl)oxy]phenyl}-2-[4-(3-fluorophenyl)-4-hydroxypiperidin-1-yl]ethanone: 0.98 g (95%) as a yellow solid.

¹H NMR (300 MHz, DMSO-d₆) δ=7.40-7.25 (m, 3H), 7.20 (s, 1H), 7.06-6.99 (m, 1H), 6.52 (s, 1H), 4.92 (s, 1H), 4.85-4.82 (m, 1H), 4.72 (s, 1H), 2.90-2.30 (m, 6H), 2.21 (s, 3H), 2.14 (s, 3H), 2.04-1.85 (m, 2H), 1.60-1.50 (m, 2H), 1.34-1.20 (m, 3H), 1.07 (d, J=7.1 Hz, 18H) ppm.

MS (ESI); (M+H)⁺=516.31

16-E: 4-(3-Fluorophenyl)-1-[2-hydroxy-2-(4-hydroxy-2,5-dimethylphenyl)ethyl]-piperidin-4-ol

By the procedures of example 2, 1-(2-{2,5-dimethyl-4-[(triisopropylsilyl)oxy]phenyl}-2-hydroxyethyl)-4-(3-fluorophenyl)piperidin-4-ol was converted to the title compound obtained as a white solid in 55% (372 mg) after recrystallization from 2-propanol.

¹H NMR (270 MHz, DMSO-d₆) δ=8.95 (s, 1H), 7.41-7.25 (m, 3H), 7.10 (s, 1H), 7.06-6.99 (m, 1H), 6.51 (s, 1H), 4.92 (s, 1H), 4.85-4.77 (m, 1H), 4.60 (s, 1H), 2.81-2.29 (m, 6H), 2.16 (s, 3H), 2.07 (s, 3H), 2.00-1.90 (m, 2H), 1.59-1.53 (m, 2H) ppm.

MS (ESI); (M+H)⁺=360.16, (M−H)⁻=358.22

m.p. 192.7° C.

IR (KBr); 3197, 1616 cm⁻¹

### Example 17

### 1-[2-Hydroxy-2-(4-hydroxy-2,5-dimethylphenyl)ethyl]-4-(6-methoxypyridin-3-yl)piperidin-4-ol

17-A: 1-{2,5-Dimethyl-4-[(triisopropylsilyl)oxy]phenyl}-2-[4-hydroxy-4-(6-methoxypyridin-3-yl)piperidin-1-yl]ethanone

The title compound was prepared according to the procedure described in Example 1 from 2-chloro-1-{2,5-dimethyl-4-[(triisopropylsilyl)oxy]phenyl}ethanone and 4-(6-methoxypyridin-3-yl)piperidin-4-ol dihydrochloride: 1.56 g (99%) as a black solid.

¹H NMR (270 MHz, DMSO-d₆) δ=8.23 (d, J=2.3 Hz, 1H), 7.76 (dd, J=8.6, 2.3 Hz, 1H), 7.73 (s, 1H), 6.75 (d, J=8.6 Hz, 1H), 6.63 (s, 1H), 4.89 (s, 1H), 3.82 (s, 3H), 3.71 (s, 2H), 2.72-2.45 (m, 4H), 2.37 (s, 3H), 2.20 (s, 3H), 1.98-1.86 (m, 2H), 1.

MS (ESI); (M+H)⁺=527.3663-1.58 (m, 2H), 1.39-1.27 (m, 3H), 1.08 (d, J=7.4 Hz, 18H) ppm.

17-B: 1-(2-{2,5-Dimethyl-4-[(triisopropylsilyl)oxy]phenyl}-2-hydroxyethyl)-4-(6-methoxypyridin-3-yl)piperidin-4-ol

The title compound was prepared according to the procedure described in Example 1 from 1-{2,5-dimethyl-4-[(triisopropylsilyl)oxy]phenyl}-2-[4-hydroxy-4-(6-methoxypyridin-3-yl)piperidin-1-yl]ethanone: 1.38 g (87%) as a black solid.

¹H NMR (300 MHz, DMSO-d₆) δ=8.23 (d, J=2.6 Hz, 1H), 7.77 (dd, J=8.6, 2.6 Hz, 1H), 7.20 (s, 1H), 6.76 (d, J=8.6 Hz, 1H), 6.51 (s, 1H), 5.76 (s, 1H), 4.87-4.70 (m, 2H), 3.83 (s, 3H), 2.77-2.28 (m, 6H), 2.20 (s, 3H), 2.14 (s, 3H), 1.99-1.87 (m, 2H), 1.63-1.58 (m, 2H), 1.33-1.17 (m, 3H), 1.07 (d, J=6.2 Hz, 18H) ppm.

MS (ES); (M+H)⁺=529.34

17-C: 1-[2-Hydroxy-2-(4-hydroxy-2,5-dimethylphenyl)ethyl]-4-(6-methoxypyridin-3-yl)piperidin-4-ol

By the procedures of example 2, 1-(2-{2,5-dimethyl-4-[(triisopropylsilyl)oxy]phenyl}-2-hydroxyethyl)-4-(6-methoxypyridin-3-yl)piperidin-4-ol was converted to the title compound obtained as a white solid in 42% (407 mg) after recrystallization from 2-propanol.

¹H NMR (270 MHz, DMSO-d₆) δ=8.94 (s, 1H), 8.24 (d, J=2.1 Hz, 1H), 7.78 (dd, J=8.6, 2.1 Hz, 1H), 7.10 (s, 1H), 6.76 (d, J=8.6 Hz, 1H), 6.50 (s, 1H), 4.85 (s, 1H), 4.84-4.79 (m, 1H), 4.60 (s, 1H), 3.82 (s, 3H), 2.75-2.28 (m, 6H), 2.16 (s, 3H), 2.07 (s, 3H), 2.00-1.90 (m, 2H), 1.66-1.58 (m, 2H) ppm.

MS (ESI); (M+H)⁺=373.17, (M−H)⁻=371.23

m.p. 156.5° C.

IR (KBr); 3282, 3165, 1607 cm⁻¹

### Example 18

### 4-(6-Ethoxypyridin-3-yl)-1-[2-hydroxy-2-(4-hydroxy-3-methylphenyl)ethyl]piperidin-4-ol

18-A: tert-Butyl 4-(6-ethoxypyridin-3-yl)-4-hydroxypiperidine-1-carboxylate

To a stirred solution of 5-bromo-2-ethoxypyridine (Yakugaku Zasshi, 1952, 72, 381) (5.02 g, 24.8 mmol) in diethylether (90 mL) was added dropwise n-butyllithium (1.56 M, 15.9 mL, 24.8 mmol) at −78° C. under nitrogen and the mixture was stirred for 50 minutes at −78° C. To the mixture, a solution of tert-butyl 4-oxopiperidine-1-carboxylate (4.49 g, 22.5 mmol) in diethylether (10 mL) was added at −78° C. The mixture was stirred at −78° C. for 2 hours and at room temperature for 3 hours. The mixture was treated with H₂O and extracted with ethyl acetate. The combined organic layer was dried and evaporated. The residue was purified by chromatography on silica gel, eluting with triethylamine/ethyl acetate/hexane (0.05:1:2 v/v/v), to afford the titled compound as a yellow oil (3.50 g, 48%).

¹H NMR (300 MHz, CDCl₃) δ=8.23 (dd, J=2.6, 0.7 Hz, 1H), 7.68 (dd, J=8.8, 2.6 Hz, 1H), 6.71 (dd, J=8.8, 0.7 Hz, 1H), 4.34 (q, J=7.1 Hz, 2H), 4.00 (br.s, 2H), 3.16-3.30 (m, 2H), 2.02-1.86 (m, 2H), 1.80-1.70 (m, 2H), 1.48 (s, 9H), 1.39 (t, J=7.1 Hz, 3H) ppm.

MS (EI); M⁺=322

18-B: 4-(6-Ethoxypyridin-3-yl)piperidin-4-ol dihydrochloride

Hydrogen chloride (10 mL, 40 mmol), 4.0 M solution in ethyl acetate, was added to a solution of tert-butyl 4-(6-ethoxypyridin-3-yl)-4-hydroxypiperidine-1-carboxylate (3.50 g, 10.9 mmol) in ethyl acetate (40 mL). The mixture was stirred at 50° C. for 1 hour. The precipitate was collected by filtration to afford the title compound as a yellow solid (3.09 g, 96%).

¹H NMR (300 MHz, DMSO) δ=9.34 (br.s, 1H), 9.21 (br.s, 1H), 8.23 (d, J=2.6 Hz, 1H), 7.89 (dd, J=8.8, 2.6 Hz, 1H), 6.97 (d, J=8.8 Hz, 1H), 6.36 (br.s, 2H), 4.34 (q, J=7.0 Hz, 2H), 3.30-1.75 (m, 8H), 1.33 (t, J=7.0 Hz, 3H) ppm.

MS (EI); M⁺=295

18-C: 2-[4-(6-Ethoxypyridin-3-yl)-4-hydroxypiperidin-1-yl]-1-{3-methyl-4-[(triisopropylsilyl)oxy]phenyl}ethanone

The title compound was prepared according to the procedure described in Example 1 from 2-bromo-1-{3-methyl-4-[(triisopropylsilyl)oxy]phenyl}-ethanone and 4-(6-Ethoxypyridin-3-yl)piperidin-4-ol dihydrochloride: 1.01 g (96%) as a yellow solid.

¹H NMR (300 MHz, DMSO-d₆) δ=8.21 (d, J=2.6 Hz, 1H), 7.84-7.79 (m, 2H), 7.76 (dd, J=8.6, 2.6 Hz, 1H), 6.88 (d, J=8.4 Hz, 1H), 6.72 (d, J=8.6 Hz, 1 H), 4.90 (s, 1H), 4.27 (q, J=7.0 Hz, 2H), 3.78 (s, 2H), 3.17-2.56 (m, 4H), 2.24 (s, 3H), 2.05-1.58 (m, 4H), 1.40-1.27 (m, 6H), 1.08 (d, J=7.3 Hz, 18H) ppm.

MS (ESI); (M+H)⁺=527.32

18-D: 4-(6-Ethoxypyridin-3-yl)-1-(2-hydroxy-2-{3-methyl-4-[(triisopropylsilyl)oxy]-phenyl}ethyl)piperidin-4-ol

The title compound was prepared according to the procedure described in Example 1 from 2-[4-(6-ethoxypyridin-3-yl)-4-hydroxypiperidin-1-yl]-1-{3-methyl-4-[(triisopropylsilyl)oxy]phenyl}ethanone: 840 mg (79%) as a yellow solid.

¹H NMR (300 MHz, DMSO-d₆) δ=8.21 (d, J=2.6 Hz, 1H), 7.75 (d, J=8.6, 2.6 Hz, 1H), 7.13-7.02 (m, 2H), 6.80-6.68 (m, 2H), 4.84 (s, 1H), 4.78-4.76 (m, 1H), 4.62 (br.s, 1H), 4.27 (q, J=7.0 Hz, 2H), 2.75-2.28 (m, 6H), 2.18 (s, 3H), 2.00-1.89 (m, 2H), 1.62-1.57 (m, 2H), 1.31 (q, J=7.0 Hz, 2H), 1.33-1.16 (m, 3H), 1.07 (d, J=7.1 Hz, 18H) ppm.

MS (ESI); (M+H)⁺=529.32

18-E: 4-(6-Ethoxypyridin-3-yl)-1-[2-hydroxy-2-(4-hydroxy-3-methylphenyl)ethyl]-piperidin-4-ol

By the procedures of example 2, 4-(6-ethoxypyridin-3-yl)-1-(2-hydroxy-2-{(3-methyl-4-[(triisopropylsilyl)oxy]phenyl}ethyl)piperidin-4-ol was converted to the title compound obtained as a white solid in 51% (300 mg) after recrystallization from 2-propanol-diisopropylether.

¹H NMR (270 MHz, DMSO-d₆) δ=9.08 (s, 1H), 8.21 (d, J=2.5 Hz, 1H), 7.76 (d, J=8.5, 2.5 Hz, 1H), 7.02 (d, J=1.8 Hz, 1H), 6.95 (dd, J=8.1, 1.8 Hz, 1H), 6.72 (d, J=8.5 Hz, 1H), 6.69 (d, J=8.1 Hz, 1H), 4.83 (s, 1H), 4.65 (s, 1if), 4.55 (br.s, 1H), 4.27 (t, J=7.1 Hz, 3H), 2.78-2.32 (m, 6H), 2.10 (s, 3H), 2.00-1.82 (m, 2H), 1.62-1.57 (m, 2H), 1.30 (q, J=7.1 Hz, 2H) ppm.

MS (ESI); (M+H)⁺=373.17, (M−H)⁻=371.23

m.p. 189.3° C.

IR (KBr); 3300, 1611 cm⁻¹

### Example 19

### 1-[2-Hydroxy-2-(4-hydroxy-2,5-dimethylphenyl)ethyl]-4-{4-(methoxymethyl)phenyl]piperidin-4-ol

19-A: 1-{2,5-Dimethyl-4-[(triisopropylsilyl)oxy]phenyl}-2-{4-hydroxy-4-[4-(methoxymethyl)phenyl]piperidin-1-yl}ethanone

The title compound was prepared according to the procedure described in Example 1 from 1-{2,5-dimethyl-4-[(triisopropylsilyl)oxy]phenyl}-2-(4-hydroxy-4-[4-(methoxymethyl)phenyl]piperidin-1-yl}ethanone: 1.05 g (97%) as a yellow solid.

¹H NMR (270 MHz, DMSO-d₆) δ=7.72 (s, 1H), 7.43 (d, J=8.2 Hz, 2H), 7.23 (d, J=8.2 Hz, 2H), 6.62 (s, 1H), 4.77 (s, 1H), 4.35 (s, 2H), 3.67 (s, 2H), 3.25 (s, 3H), 2.70-2.31 (m, 4H), 2.35 (s, 3H), 2.18 (s, 3H), 1.97-1.91 (m, 2H), 1.59-1.47 (m, 2H), 5 1.40-1.20 (m, 3H), 1.06 (d, J=7.2 Hz, 18H) ppm.

MS (ESI); (M+H)⁺=540.33

19-B: 1-(2-{2,5-Dimethyl-4-[(triisopropylsilyl)oxy]phenyl}-2-hydroxyethyl)-4-[4-(methoxymethyl)phenyl]piperidin-4-ol

The title compound was prepared according to the procedure described in Example 1 from 1-{2,5-Dimethyl-4-[(triisopropylsilyl)oxy]phenyl}-2-{4-hydroxy-4-[4-(methoxymethyl)phenyl]piperidin-1-yl}ethanone: 994 mg (90%) as a yellow solid.

¹H NMR (300 MHz, DMSO-d₆) δ=7.45 (d, J=8.1 Hz, 2H), 7.24 (d, J=8.1 Hz, 2H), 7.19 (s, 1H), 6.50 (s, 1H), 4.86-4.66 (m, 3H), 4.36 (s, 2H), 3.26 (s, 3H), 2.79-2.27 (m, 6H), 2.19 (s, 3H), 2.13 (s, 3H), 2.03-1.84 (m, 2H), 1.60-1.49 (m, 2H), 1.36-1.18 (m, 3H), 1.06 (d, J=6.4 Hz, 18H) ppm.

MS (ESI); (M+H)⁺=542.35

19-C: 1-[2-Hydroxy-2-(4-hydroxy-2,5-dimethylphenyl)ethyl]-4-[4-(methoxymethyl)-phenyl]piperidin-4-ol

By the procedures of example 2, 1-(2-{2,5-dimethyl-4-[(triisopropylsilyl)oxy]phenyl}-2-hydroxyethyl)-4-[4-(methoxymethyl)phenyl]piperidin-4-ol was converted to the title compound obtained as a white solid in 60% (465 mg) after recrystallization from 2-propanol.

¹H NMR (270 MHz, DMSO-d₆) δ=8.94 (s, 1H), 7.46 (d, J=8.2 Hz, 2H), 7.25 (d, J=8.1 Hz, 2H), 7.10 (s, 1H), 6.50 (s, 1H), 4.81 (d, J=7.7 Hz, 1H), 4.75 (s, 1H), 4.59 (br.s, 1H), 4.37 (s, 2H), 3.27 (s, 3H), 2.80-2.27 (m, 6H), 2.16 (s, 3H), 2.07 (s, 3H), 2.01-1.88 (m, 2H), 1.59-1.54 (m, 2H) ppm.

MS (ESI); (M+H)⁺=386.18, (M−H)⁻=384.23

m.p. 166.3° C.

IR (KBr); 3343, 1620 cm⁻¹

### Example 20

### 1-[2-(3-Ethylhydroxyphenyl)-2-hydroxyethyl]-4-(6-methoxypyridin-3-yl)piperidin-4-ol hydrochloride

20-A: 2-Chloro-1-(3-ethyl-4-hydroxyphenyl)ethanone

The title compound was prepared according to the procedure described in Example 16 from 2-ethylphenol: 2.02 g (46%) as a white-red solid.

¹H NMR (300 MHz, CDCl₃) δ=7.81 (d, J=2.2 Hz, 1H), 7.74 (dd, J=8.4, 2.2 Hz, 1H), 6.85 (d, J=8.4 Hz, 1H), 5.87 (s, 1H), 4.67 (s, 2H), 2.69 (q, J=7.5 Hz, 2H), 1.26 (t, J=7.5 Hz, 3H) ppm.

MS (EI); M⁺=198

20-B: 2-Chloro-1-{3-ethyl-4-[(triisopropylsilyl)oxy]phenyl}ethanone

The title compound was prepared according to the procedure described in Example 2 from 2-chloro-1-(3-ethyl-4-hydroxyphenyl)ethanone: 3.09 g (86%) as a yellow oil.

¹H NMR (300 MHz, CDCl₃) δ=7.80 (d, J=2.4 Hz, 1H), 7.71 (dd, J=8.4, 2.4 Hz, 1H), 6.82 (d, J=8.4 Hz, 1H), 4.66 (s, 2H), 2.69 (q, J=7.5 Hz, 2H), 1.40-1.27 (m, 3H), 1.22 (t, J=7.5 Hz, 3H), 1.12 (d, J=7.1 Hz, 18H) ppm.

MS (EI); M⁺=354

20-C: 1-(2-{3-Ethyl-4-[(triisopropylsilyl)oxy]phenyl}-2-hydroxyethyl)-4-(6-methoxypyridin-3-yl)piperidin-4-ol

The title compound was prepared according to the procedure described in Example 1 from 2-chloro-1-{3-ethyl-4-[(triisopropylsilyl)oxy]phenyl}ethanone and 4-(6-methoxypyridin-3-yl)piperidin-4-ol dihydrochloride: 987 mg (93%) as a yellow oil.

¹H NMR (270 MHz, DMSO-d₆) δ=8.23 (d, J=2.5 Hz, 1H), 7.77 (dd, J=8.6, 2.5 Hz, 1H), 7.13 (d, J=2.0 Hz, 1H), 7.05 (dd, J=8.4, 2.0 Hz, 1H), 6.75 (d, J=8.6 Hz, 1H), 6.72 (d, J=8.4 Hz, 1H), 4.86 (s, 1H), 4.79 (s, 1H), 4.63 (br.s, 1H), 3.82 (s, 3H), 2.72-2.34 (m, 8H), 2.02-1.76 (m, 2H), 1.62-1.57 (m, 2H), 1.36-1.11 (m, 6H), 1.07 (d, J=7.3 Hz, 18H) ppm.

MS (ESI); (M+H)⁺=529.31

20-D: 1-[2-(3-Ethyl-4-hydroxyphenyl)-2-hydroxyethyl]-4-(6-methoxypyridin-3-yl-piperidin-4-ol

The title compound was prepared according to the procedure described in Example 2 from 1-(2-{3-ethyl-4-[(triisopropylsilyl)oxy]phenyl}-2-hydroxyethyl)-4-(6-methoxypyridin-3-yl)piperidin-4-ol: 376 mg (50%) as a yellow oil.

¹H NMR (270 MHz, DMSO-d₆) δ=9.08 (s, 1H), 8.23 (d, J=2.6 Hz, 1H), 7.77 (dd, J=8.7, 2.6 Hz, 1H), 7.03 (d, J=2.0 Hz, 1H), 6.96 (dd, J=8.3, 2.0 Hz, 1H), 6.76 (d, J=8.7 Hz, 1H), 6.70 (d, J=8.3 Hz, 1H), 4.87 (s, 1H), 4.69 (s, 1H), 4.61-4.56 (m, 1H), 3.83 (s, 3H), 2.78-2.33 (m, 8H), 2.02-1.86 (m, 2H), 1.63-1.57 (m, 2H), 1.12 (t, J=7.6 Hz, 3H) ppm.

20-E: 1-[2-(3-Ethyl-4-hydroxyphenyl)-2-hydroxyethyl]-4-(6-methoxypyridin-3-yl)-piperidin-4-ol hydrochloride

By the procedures of example 1, 1-[2-(3-ethyl-4-hydroxyphenyl)-2-hydroxyethyl]-4-(6-methoxypyridin-3-yl)piperidin-4-ol was converted to the title compound obtained as a white amorphous in 97% (400 mg) after crystallization from ethanol-hexane.

¹H NMR (270 MHz, DMSO-d₆) δ=9.65 (br.s, 1H), 9.32 (s, 1H), 8.26 (br.s, 1H), 7.78 (d, J=8.7 Hz, 1H), 7.12 (s, 1H), 7.05 (d, J=8.1 Hz, 1H), 6.84-6.76 (m, 2H), 5.92 (br.s, 1H), 5.49 (s, 1H), 4.96 (s, 1H), 3.84 (s, 3H), 3.65-1.77 (m, 12H), 1.14 (t, J=7.6 Hz, 3H) ppm.

MS (ESI); (M+H)⁺=373.12, (M−H)⁻=371.21

IR (KBr); 3263, 1609 cm⁻¹

### Example 21

### 1-[2-(2-Fluoro-4-hydroxy-5-methylphenyl)-2-hydroxyethyl]-4-(6-methoxypyridin-3-yl)piperidin-4-ol

21-A: 1-(2-Fluoro-4-hydroxy-5-methylphenyl)ethanone

The title compound was prepared according to the procedure described in Example 16 from 5-fluoro-2-methylphenol (Tetrahedron, 1959, 6, 315): 856 mg (43%) as a yellow oil.

¹H NMR (270 MHz, CDCl₃) δ=7.71 (d, J=8.6 Hz, 1H), 6.57 (d, J=12.0 Hz, 1H), 5.99 (s, 1H), 2.59 (d, J=5.3 Hz, 3H), 2.23 (s, 3H) ppm.

MS (EI); M⁺=168

21-B: 1-{2-Fluoro-5-methyl-4-[(triisopropylsilyl)oxy]phenyl}ethanone

The title compound was prepared according to the procedure described in Example 2 from 1-(2-fluoro-4-hydroxy-5-methylphenyl)ethanone: 1.39 g (85%) as a yellow oil.

¹H NMR (270 MHz, CDCl₃) δ=7.70 (d, J=9.0 Hz, 1H), 6.50 (d, J=12.7 Hz, 1H), 2.58 (d, J=5.3 Hz, 3H), 2.20 (s, 3H), 1.39-1.25 (m, 3H), 1.12 (d, J=7.1 Hz, 18H) ppm.

MS (EI); M⁺=324

21-C: 2-Bromo-1-{2-fluoro-5-methyl-4-[(triisopropylsilyl)oxy]phenyl}ethanone

The title compound was prepared according to the procedure described in Example 4 from 1-{2-fluoro-5-methyl-4-[(triisopropylsilyl)oxy]phenyl}ethanone: 1.9 g (quant.) as a yellow oil.

¹H NMR (270 MHz, CDCl₃) δ=7.76 (d, J=8.9 Hz, 1H), 6.52 (d, J=12.9 Hz, 1H), 4.47 (d, J=2.0 Hz, 2H), 2.21 (s, 3H), 1.39-1.23 (m, 3H), 1.12 (d, J=7.3 Hz, 18H) ppm.

MS (EI); M⁺=402, 404

21-D: 1-{2-Fluoro-5-methyl-4-[(triisopropylsilyl)oxy]phenyl}-2-[4-hydroxy-4-(6-methoxypyridin-3-yl)piperidin-1-yl]ethanone

The title compound was prepared according to the procedure described in Example 1 from 2-bromo-1-{2-fluoro-5-methyl-4-[(triisopropylsilyl)oxy]phenyl}ethanone and 4-(6-methoxypyridin-3-yl)piperidin-4-ol dihydrochloride: 1.05 g (99%) as a yellow oil.

¹H NMR (270 MHz, DMSO-d₆) δ=8.22 (d, J=2.6 Hz, 1H), 7.76 (dd, J=8.6, 2.6 Hz, 1H), 7.68 (d, J=8.6 Hz, 1H), 6.75 (d, J=8.6 Hz, 1H), 6.63 (d, J=12.5 Hz, 1H), 4.90 (s, 1H), 3.82 (s, 3H), 3.71 (s, 2H), 2.65-1.57 (m, 8H), 2.29 (s, 3H), 1.42-1.31 (m, 3H), 1.08 (d, J=7.4 Hz, 18H) ppm.

MS (ESI); (M+H)⁺=531.28

21-E: 1-(2-{2-Fluoro-5-methyl-4-[(triisopropylsilyl)oxy]phenyl}-2-hydroxyethyl)-4-(6-methoxypyridin-3-yl)piperidin-4-ol

The title compound was prepared according to the procedure described in Example 1 from 1-{2-fluoro-5-methyl-4-[(triisopropylsilyl)oxy]phenyl}-2-[4-hydroxy-4-(6-methoxypyridin-3-yl)piperidin-1-yl]ethanone: 354 mg (33%) as a yellow oil.

¹H NMR (270 MHz, DMSO-d₆) δ=8.21 (br.s, 1H), 7.75 (d, J=8.6 Hz, 1H), 7.26 (d, J=8.9 Hz, 1H), 6.75 (d, J=8.6 Hz, 1H), 6.48 (d, J=11.2 Hz, 1H), 4.92-4.83 (m, 3H), 3.82 (s, 3H), 2.67-1.56 (m, 10H), 2.15 (s, 3H), 1.34-1.25 (m, 3H), 1.07 (d, J=7.4 Hz, 18H) ppm.

MS (ESI); (M+H)⁺=533.29

21-F: 1-[2-(2-Fluoro-4-hydroxy-5-methylphenyl)-2-hydroxyethyl]-4-(6-methoxypyridin-3-yl)piperidin-4-ol

By the procedures of example 2, 1-(2-{2-fluoro-5-methyl-4-[(triisopropylsilyl)oxy]phenyl}-2-hydroxyethyl)-4-(6-methoxypyridin-3-yl)piperidin-4-ol was converted to the title compound obtained as a white amorphous in 100% (260 mg) after crystallization from 2-propanol.

¹H NMR (270 MHz, DMSO-d₆) δ=9.59 (s, 1H), 8.22 (d, J=2.6 Hz, 1H), 7.76 (dd, J=8.7, 2.6 Hz, 1H), 7.15 (d, J=8.7 Hz, 1H), 6.75 (d, J=8.7 Hz, 1H), 6.48 (d, J=11.9 Hz, 1H), 4.92-4.83 (m, 3H), 3.82 (s, 3H), 2.72-2.36 (m, 6H), 2.07 (s, 3H), 1.95-1.56 (m, 4H) ppm.

MS (ESI); (M+H)⁺=377.13, (M−H)⁻=375.20

m.p. 183.6° C.

IR (KBr); 3260, 1614 cm⁻¹

### Example 22

### 1-[2-(2-Fluoro-4-hydroxy-5-methylphenyl)-2-hydroxyethyl]-4-(3-fluorophenyl)piperidin-4-ol

22-A: 2-Chloro-1-{2-fluoro-5-methyl-4-[(triisopropylsilyl)oxy]phenyl}ethanone

The title compound was prepared according to the procedure described in Example 16 from 5-fluoro-2-methylphenol: 2.7 g (27%) as a brown oil.

¹H NMR (270 MHz, CDCl₃) δ=7.78 (d, J=8.7 Hz, 1H), 6.52 (d, J=13.0 Hz, 1H), 4.68 (d, J=2.9 Hz, 2H), 2.22 (s, 3H), 1.39-1.17 (m, 3H), 1.12 (d, J=7.3 Hz, 18H) ppm.

MS (EI); M⁺=358

22-B: 1-{2-Fluoro-5-methyl-4-[(triisopropylsilyl)oxy]phenyl}-2-[4-(3-fluorophenyl)-4-hydroxypiperidin-1-yl]ethanone

The title compound was prepared according to the procedure described in Example CJ-26562-27 from 2-chloro-1-{2-fluoro-5-methyl-4-[(triisopropylsilyl)oxy]phenyl}ethanone: 144 mg (14%) as a brown solid.

¹H NMR (270 MHz, DMSO-d₆) δ=7.76-7.54 (m, 1H), 7.40-7.17 (m, 3H), 7.07-6.94 (m, 1H), 6.68-6.54 (m, 1H), 4.93 (s, 1H), 3.72-3.60 (m, 2H), 2.71-1.81 (m, 6H), 2.17 (s, 3H), 1.59-1.48 (m, 2H), 1.45-1.24 (m, 3H), 1.07 (d, J=7.4 Hz, 18H) ppm.

MS (ESI); (M+H)⁺=518.24

22-C: 1-[2-(2-Fluoro-4-hydroxy-5-methylphenyl)-2-hydroxyethyl]-4-(3-fluorophenyl)piperidin-4-ol

By the procedures of example 2, 1-{2-fluoro-5-methyl-4-[(triisopropylsilyl)oxy]phenyl}-2-[4-(3-fluorophenyl)-4-hydroxypiperidin-1-yl]ethanone was converted to the title compound obtained as a white solid in 76% (77 mg) after crystallization from 2-propanol-diisopropylether.

¹H NMR (300 MHz, DMSO-d₆) δ=9.63 (br.s, 1H), 7.37-7.25 (m, 3H), 7.16 (d, J=8.6 Hz, 1H), 7.06-7.01 (m, 1H), 6.56-6.48 (m, 1H), 4.93-4.89 (m, 3H), 2.75-2.30 (m, 6H), 2.08 (s, 3H), 1.97-1.77 (m, 2H), 1.58-1.53 (m, 2H) ppm.

MS (ESI); (M+H)⁺=364.11, (M−H)⁻=362.17

m.p. 161.7° C.

IR (KBr); 3377, 3202, 1622 cm⁻¹

### Example 23

### 4-(6-Fluoro-5-methoxypyridin-2-yl)-1-[2-hydroxy-2-(4-hydroxy-3-methylphenyl)ethyl]piperidin-4-ol

23-A: 6-Bromo-2-fluoropyridin-3-ol

To a stirred solution of 2-fluoropyridin-3-ol (J. Labelled Compound. Radiopharm., 1998, 41, 451) (3.81 g, 33.7 mmol) and sodium acetate (2.76 g, 33.7 mmol) in acetic acid (30 mL) was added bromine (1.74 mL, 33.7 mmol) at 0° C., and the mixture was stirred at room temperature for 3.5 hours. The mixture was poured onto ice-aq.sodium hydroxide and extracted with ethyl acetate. The combined organic layer was dried and evaporated to afford the titled compound as a yellow solid (4.67 g, 72%).

¹H NMR (270 MHz, DMSO-d₆) δ=7.29 (d, J=8.2 Hz, 1H), 7.28 (s, 1H), 7.23 (d, J=8.2 Hz, 1H) ppm.

MS (EI); M⁺=191, 193

23-B: 6-Bromo-2-fluoro-3-methoxypyridine

To a stirred solution of 6-bromo-2-fluoropyridin-3-ol (4.67 g, 24.3 mmol) and sodium methoxide (1.38 g, 25.5 mmol) in N,N-dimethylformamide (50 mL) was added methyl iodide (1.59 mL, 25.5 mmol) at 0° C., and the mixture was stirred at room temperature for 12 hours. The mixture was treated with H₂O and extracted with ethyl acetate. The combined organic layer was dried and evaporated. The residue was purified by chromatography on silica gel, eluting with ethyl acetate/hexane (1:5 v/v), to afford the titled compound as a yellow oil (2.43 g, 49%).

¹H NMR (270 MHz, CDCl₃) δ=7.32-7.26 (m, 1H), 7.22-7.15 (m, 1H), 3.90 (s, 3H) ppm.

MS (EI); M⁺=205, 207

23-C: tert-Butyl 4-(6-fluoro-5-methoxypyridin-2-yl)-4-hydroxypiperidine-1-carboxylate

The title compound was prepared according to the procedure described in Example 18 from 6-bromo-2-fluoro-3-methoxypyridine: 948 mg (49%) as a colorless oil.

¹H NMR (300 MHz, CDCl₃) δ=7.31 (dd, J=9.9, 8.3 Hz, 1H), 7.18 (dd, J=8.3, 0.9 Hz, 1H), 4.15-3.95 (m, 2H), 3.91 (s, 3H), 3.32-3.15 (m, 2H), 2.02-1.80 (m, 2H), 1.70-1.53 (m, 2H), 1.48 (s, 9H) ppm.

23-C: 4-(6-Fluoro-5-methoxypyridin-2-yl)piperidin-4-ol dihydrochloride

The title compound was prepared according to the procedure described in Example 18 from tert-butyl 4-(6-fluoro-5-methoxypyridin-2-yl)-4-hydroxypiperidine-1-carboxylate: 623 mg (72%) as a white solid.

¹H NMR (300 MHz, DMSO-d₆) δ=9.24-9.12 (m, 1H), 8.78 (br.s, 1H), 7.69 (dd, J=10.6, 8.2 Hz, 1H), 7.53 (d, J=8.2 Hz, 1H), 5.74 (br.s, 1H), 3.88 (s, 3H), 3.20-3.10 (m, 4H), 2.30-2.16 (m, 2H), 1.80-1.66 (m, 2H) ppm.

MS (ESI); (M+H)⁺=227.01

23-D: 2-[4-(6-Fluoro-5-methoxypyridin-2-yl)-4-hydroxypiperidin-1-yl]-1-{3-methyl-4-[(triisopropylsilyl)oxy]phenyl}ethanone

The title compound was prepared according to the procedure described in Example CJ-26562-27 from 2-bromo-1-{3-methyl[(triisopropylsilyl)oxy]phenyl}ethanone and 4-(6-fluoro-5-methoxypyridin-2-yl)piperidin-4-ol dihydrochloride: 699 mg (quant.) as a yellow solid.

¹H NMR (270 MHz, DMSO-d₆) δ=7.85-7.80 (m, 2H), 7.66-7.47 (m, 2H), 6.88 (d, J=8.9 Hz, 1H), 5.03 (s, 1H), 3.85 (s, 3H), 3.73 (s, 2H), 2.67-2.50 (m, 4H), 2.24 (s, 3H), 2.20-1.98 (m, 2H), 1.51-1.25 (m, 5H), 1.08 (d, J=7.2 Hz, 18H) ppm.

MS (ESI); (M+H)⁺=531.28

23-E: 4-(6-Fluoro-5-methoxypyridin-2-yl)-1-(2-hydroxy-2-{3-methyl-4-[(triisopropylsilyl)oxy]phenyl}ethyl)piperidin-4-ol

The title compound was prepared according to the procedure described in Example CJ-26562-27 from 2-[4-(6-fluoro-5-methoxypyridin-2-yl)-4-hydroxypiperidin-1-yl]-1-{3-methyl-4-[(triisopropylsilyl)oxy]phenyl}ethanone: 649 mg (94%) as a yellow solid.

¹H NMR (270 MHz, DMSO-d₆) δ=7.66-7.58 (m, 1H), 7.49 (d, J=7.9 Hz, 1H), 7.12 (s, 1H), 7.04 (d, J=8.2 Hz, 1H), 6.72 (d, J=8.2 Hz, 1H), 4.98 (s, 1H), 4.75 (s, 1H), 4.60 (br.s, 1H), 3.85 (s, 3H), 2.71-2.01 (m, 8H), 2.18 (s, 3H), 1.50-1.44 (m, 2H), 1.35-1.20 (m, 3H), 1.07 (d, J=7.2 Hz, 18H) ppm.

MS (ESI); (M+H)⁺=533.29

23-F: 4-(6-Fluoro-5-methoxypyridin-2-yl)-1-[2-hydroxy-2-(4-hydroxy-3-methylphenyl)ethyl]piperidin-4-ol

By the procedures of example 2, 4-(6-fluoro-5-methoxypyridin-2-yl)-1-(2-hydroxy-2-{3-methyl-4-[(triisopropylsilyl)oxy]phenyl}ethyl)piperidin-4-ol was converted to the title compound obtained as a white solid in 60% (291 mg) after rerystallization from 2-propanol.

¹H NMR (270 MHz, DMSO-d₆) δ=9.08 (s, 1H), 7.66-7.58 (m, 1H), 7.49 (d, J=8.1 Hz, 1H), 7.02 (s, 1H), 6.94 (d, J=8.1 Hz, 1H), 6.69 (d, J=8.1 Hz, 1H), 4.99 (s, 1H), 4.63 (s, 1H), 4.55 (s, 1H), 3.86 (s, 3H), 2.76-2.66 (m, 2H), 2.54-2.30 (m, 4H), 2.10 (s, 3H), 2.12-1.96 (m, 2H), 1.50-1.44 (m, 2H) ppm.

MS (ESI); (M+H)⁺=377.13, (M−H)⁻=375.20

m.p. 172.3° C.

IR (KBr); 3382, 3317 cm⁻¹

### Example 24

### 1-[2-Hydroxy-2-(4-hydroxy-3-methylphenyl)ethyl]-4-(6-propoxypyridin-3-yl)piperidin-4-ol

24-A: 5-Bromo-2-propoxypyridine

To a stirred solution of sodium (591 mg, 24.6 mmol) in 2-propanol (20 mL) was added a solution of 5-bromo-2-nitropyridine (5 g, 24.6 mmol) in 2-propanol (10 mL) at room temperature and the mixture was stirred under reflux for 2.5 hours. After all solvents were removed, the residue was diluted with dichloromethane and H₂O, and extracted with dichloromethane. The combined organic layer was dried and evaporated. The residue was purified by chromatography on silica gel, eluting with ethyl acetate/hexane (1:10, v/v), to afford the titled compound as a colorless oil (3.84 g, 72%).

¹H NMR (270 MHz, CDCl₃) δ=8.17 (d, J=2.6 Hz, 1H), 7.63 (dd, J=8.7, 2.6 Hz, 1H), 6.64 (d, J=8.7 Hz, 1H), 4.21 (t, J=6.6 Hz, 2H), 1.84-1.70 (m, 2H), 1.01 (t, J=7.4 Hz, 3H) ppm.

MS (EI); M⁺=215, 217

24-B: tert-Butyl 4-hydroxy-4-(6-propoxypyridin-3-yl)piperidine-1-carboxylate

The title compound was prepared according to the procedure described in Example 18 from 5-bromo-2-propoxypyridine: 2.57 g (75%) as a yellow oil.

¹H NMR (270 MHz, CDCl₃) δ=8.23 (d, J=2.6 Hz, 1H), 7.68 (dd, J=8.7, 2.6 Hz, 1H), 6.72 (d, J=8.7 Hz, 1H), 4.24 (t, J=6.8 Hz, 2H), 4.00 (br.s, 2H), 3.29-3.19 (m, 2H), 2.04-1.70 (m, 6H), 1.48 (s, 9H), 1.02 (t, J=7.4 Hz, 3H) ppm.

MS (E); M⁺=336

24-C: 4-(6-Propoxypyridin-3-yl)piperidin-4-ol dihydrochloride

The title compound was prepared according to the procedure described in Example 18 from tert-butyl 4-hydroxy-4-(6-propoxypyridin-3-yl)piperidine-1-carboxylate: 2.5 g (quant.) as a yellow solid.

¹H NMR (300 MHz, DMSO) δ=9.35-9.00 (m, 1H), 8.22 (d, J=2.4 Hz, 1H), 7.94-7.80 (m, 1H), 7.02-6.88 (m, 1H), 4.60-4.00 (m, 4H), 3.22-3.10 (m, 4H), 2.32-2.15 (m, 2H), 1.83-1.76 (m, 2H), 1.77-1.66 (m, 2H), 0.96 (t, J=6.8 Hz, 3H) ppm.

MS (EI); M⁺=236

24-D: 2-[4-Hydroxy-4-(6-propoxypyridin-3-yl)piperidin-1-yl]-1-{3-methyl-4-[(triisopropylsilyl)oxy]phenyl}ethanone

The title compound was prepared according to the procedure described in Example CJ-26562-27 from 2-bromo-1-{3-methyl-4-[(triisopropylsilyl)oxy]phenyl}ethanone and 4-(6-propoxypyridin-3-yl)piperidin-4-ol dihydrochloride: 716 mg (quant.) as a brown oil.

¹H NMR (270 MHz, DMSO-d₆) δ=8.23-8.18 (m, 1H), 7.85-7.74 (m, 3H), 6.88 (d, J=8.2 Hz, 1H), 6.73 (d, J=8.6 Hz, 1H), 4.88 (s, 1H), 4.18 (t, J=6.6 Hz, 2H), 3.77 (s, 2H), 3.10-2.50 (m, 4H), 2.24 (s, 3H), 1.99-1.58 (m, 5H), 1.38-1.17 (m, 3H), 1.08 (d, J=7.4 Hz, 18H), 0.95 (s, 3H) ppm.

MS (ESI); (M+H)⁺=541.31

24-E: 1-(2-Hydroxy-2-{3-methyl-4-[(triisopropylsilyl)oxy]phenyl}ethyl)-4-(6-propoxypyridin-3-yl)piperidin-4-ol

The title compound was prepared according to the procedure described in Example CJ-26562-27 from 2-[4-hydroxy-4-(6-propoxypyridin-3-yl)piperidin-1-yl]-1-{3-methyl-4-[(triisopropylsilyl)oxy]phenyl}ethanone: 666 mg (94%) as a yellow oil.

¹H NMR (270 MHz, DMSO-d₆) δ=8.21 (d, J=2.1 Hz, 1H), 7.76 (dd, J=8.7, 2.1 Hz, 1H), 7.07 (d, J=8.6 Hz, 1H), 7.12 (s, 1H), 6.75-6.70 (m, 2H), 4.84 (s, 1H), 4.65-4.58 (m, 2 H), 4.18 (t, J=6.8 Hz, 2H), 2.99-2.35 (m, 6H), 2.18 (s, 3H), 1.95-1.57 (m, 6H), 1.37-1.20 (m, 3H), 1.07 (d, J=7.2 Hz, 18H), 0.95 (t, J=7.4 Hz, 3H) ppm.

MS (ESI); (M+H)⁺=543.32

24-F: 1-[2-Hydroxy-2-(4-hydroxy-3-methylphenyl)ethyl]-4-(6-propoxypyridin-3-yl)-piperidin-4-ol

By the procedures of example 2, 1-(2-hydroxy-2-{3-methyl-4-[(triisopropylsilyl)oxy]phenyl}ethyl)-4-(6-propoxypyridin-3-yl)piperidin-4-ol was converted to the title compound obtained as a white solid in 40% (200 mg) after crystallization from 2-propanol.

¹H NMR (270 MHz, DMSO-d₆) δ=9.08 (s, 1H), 8.21 (br.s, 1H), 7.76 (d, J=9.4 Hz, 1H), 7.02 (s, 1H), 6.95 (d, J=9.4 Hz, 1H), 6.73 (d, J=9.4 Hz, 1H), 6.70 (d, J=9.4 Hz, 1H), 4.84 (s, 1H), 4.65 (s, 1H), 4.56 (br.s, 1H), 4.18 (t, J=6.4 Hz, 2H), 2.75-2.30 (m, 6H), 2.10 (s, 3H), 1.95-1.85 (m, 2H), 1.75-1.54 (m, 4H), 0.95 (t, J=7.2 Hz, 3H) ppm.

MS (ESI); (M+H)⁺=387.16, (M−H)⁻=385.25

m.p. 187.4° C.

IR (KBr); 3319, 1611 cm⁻¹

### Example 25

### 1-[2-(3-chloro-4-hydroxyphenyl)-2-hydroxyethyl]-4-(6-methoxypyridin-3-yl)piperidin-4-ol hydrochloride

25-A: 1-{2-[4-(benzyloxy)-3-chlorophenyl]-2-hydroxyethyl}-4-(6-methoxypyridin-3-yl)piperidin-4-ol

To a stirred suspension of 4-(6-methoxypyridin-3-yl)piperidin-4-ol dihydrochloride (0.86 g) in tetrahydrofuran (10 ml) was added triethylamine (1.5 mL) at room temperature under nitrogen, then tetrahydrofuran solution of 1-[4-(benzyloxy)-3-chlorophenyl]-2-bromoethanone (J. Med. Chem., 23 738 (1980)) (0.73 g) was added to the mixture. The whole was stirred at room temperature 3 hours. The reaction mixture was added ethyl alcohol (5 mL) and sodium borohydride (0.5 g).

The mixture was stirred 3 hours and poured into water (100 ml) whole was extracted with ethyl acetate (50 mL×2). The combined organic layers were washed with saturated aqueous sodium chloride solution, dried over sodium sulfate, filtered and concentrated under reduced pressure. The residue was purified by chromatography on amino type silica gel, eluting with ethyl acetate/methanol (20:1 v/v), to afford the titled compound as a solid (0.6 g).

¹H NMR (300 MHz, CDCl₃) δ 8.03 (d, J=2 Hz, 1H), 7.72 (dd, J=9, 2 Hz, 1H), 7.48-7.28 (m, 6H), 7.19 (dd, J=9, 2 Hz, 1H), 6.94 (d, J=8 Hz, 1H), 6.75 (d, J=9 Hz, 1H), 5.16 (s, 2H), 4.68 (dd, J=10,42 Hz, 1H), 3.02 (brd, J=12 Hz, 1H), 2.83 (dt, J=2, 12 Hz, 1H), 2.74-2.66 (1H), 2.60-2.43 (m, 3H), 2.21-2.04 (m, 2H), 1.88-1.76 (m, 2H) ppm.

25-B: 1-[2-(3-chloro-4-hydroxyphenyl)-2-hydroxyethyl]-4-(6-methoxypyridin-3-yl)piperidin-4-ol hydrochloride

A mixture of 1-{2-[4-(benzyloxy)-3-chlorophenyl]-2-hydroxyethyl}-4-(6-methoxypyridin-3-yl)piperidin-4-ol (0.6 g), palladium 5 wt % on activated carbon (0.04 g) and methanol (50 mL) was stirred under hydrogen atmosphere (4 kg/m²) at room temperature for 5 hours. The resulting mixture was filtered through Celite, and the filtrate was concentrated. The residue was purified by chromatography on amino type silica gel, eluting with methyl alcohol/ethyl acetate (1:20 v/v), to afford the titled compound as a white solid (0.45 g). Hydrogen chloride (0.27 ml, 1.0 eq), 4.0 M solution in ethyl acetate, was added to a solution of 1-[2-(3-chloro-4-hydroxyphenyl)-2-hydroxyethyl]-4-(6-methoxypyridin-3-yl)piperidin-4-ol (0.6 g) in methyl alcohol (5 mL). The mixture was stirred for 1 hours at room temperature and concentrated in vacuo. The residue was crystallized from 2-propanol to afford the titled compound as a white solid. (0.22 g).

¹H NMR (300 MHz, DMSO-d₆) δ=10.28 (s, 1H), 9.71 (br, 1H), 8.25 (d, J=2 Hz, 1H), 7.76 (dd, J=9, 2 Hz, 1H), 7.41 (d, J=2 Hz, 1H), 7.21 (dd, J=8, 2 Hz, 1H), 7.00 (d, J=8 Hz, 1H), 6.84 (d, J=9 Hz, 1H), 6.24-6.21 (br, 1H), 5.57 (br, 2H), 5.1-5.0 (br, 1H), 3.84 (s, 3H), 3.7-3.2 (4H), 2.5-2.3 (2H), 2.0-1.8 (m, 2H) ppm

MS (ESI); (M+H)⁺=379.05, (M−H)⁻=377.13

m.p. 214.3° C.

### Example 26

### 1-[2-(3-chloro-4-hydroxyphenyl)-2-hydroxyethyl]-4-[4-(methoxymethyl)phenyl]piperidin-4-ol hydrochloride

26-A: 1-[4-(benzyloxy)-3-chlorophenyl]-2-{4-hydroxy-4-[4-(methoxymethyl)phenyl]piperidin-1-yl}ethanone

To a stirred solution of 4-[4-(methoxymethyl)phenyl]piperidin-4-ol (0.65 g) in tetrahydrofuran (5 ml) was added triethylamine (2.6 g) at room temperature under nitrogen atmosphere and tetrahydrofuran solution of 1-[4-(benzyloxy)-3-chlorophenyl]-2-bromoethanone (0.7 g). Whole was stirred at room temperature 16 hours. The reaction mixture was added water and extracted with ethyl acetate. The organic layer was washed with brine and dried over NaSO4. The residue was concentrated in vacuo to afford the titled compound as a solid (0.7 g). The crude product was used in the next step without further purification.

26-B: 1-{2-[4-(benzyloxy)-3-chlorophenyl]-2-hydroxyethyl}-4-[4-(methoxymethyl)phenyl]piperidin-4-ol

The title compound is prepared from 1-[4-(benzyloxy)-3-chlorophenyl]-2-{4-hydroxy-4-[4-(methoxymethyl)phenyl]piperidin-1-yl}ethanone (0.7 g) inseared of 1-{2-[4-(benzyloxy)-3-chlorophenyl]-2-hydroxyethyl}-4-(6-methoxypyridin-3-yl)piperidin-4-ol according to the method described in Example 1 as a solid (0.65 g).

¹H NMR (300 MHz, CDCl₃) δ=7.52-7.3 (m, 10H), 7.20 (dd, J=9, 2 Hz, 1H), 6.94 (d, J=9 Hz, 1H), 5.16 (s, 2H), 4.73 (dd, J=10, 4 Hz, 1H), 4.46 (s, 2H), 3.40 (s, 3H), 3.12-3.05 (m, 1H), 2.94-2.75 (m, 2H), 2.70-2.45 (m, 2H), 2.31-1.76 (m, 5H) ppm.

26-C: 1-[2-(3-chloro-4-hydroxyphenyl)-2-hydroxyethyl]-4-[4-(methoxymethyl)phenyl]piperidin-4-ol hydrochloride

The title compound is prepared from 1-{2-[4-(benzyloxy)-3-chlorophenyl]-2-hydroxyethyl}-4-[4-(methoxymethyl)phenyl]piperidin-4-ol (0.5 g) inseared of 1-[4-(benzyloxy)-3-fluorophenyl]-2-[4-hydroxy-4-(6-methoxypyridin-3-yl)piperidin-4-yl]ethanone according to the method described in Example 25 as a solid (0.17 g).

¹H NMR (300 MHz, CDCl₃) δ=7.50 (d, J=8 Hz, 2H), 7.39 (d, J=8 Hz, 2H), 7.35 (d, J=8 Hz, 2H), 7.18 (dd, J=8, 2 Hz, 1H), 6.98 (d, J=8 Hz, 1H), 4.69 (dd, J=11, 3 Hz, 1H), 4.46 (s, 2H), 3.40 (s, 3H), 3.02 (d, J=11 Hz, 1H), 2.85 (dt, J=3, 12 Hz, 1H), 7.77 (d, J=11 Hz, 1H), 2.61-2.35 (m, 3H), 2.25-2.08 (m, 2H), 1.85-1.75 (m, 2H) ppm.

MΣ (EΣI); (M+H)⁺=358.10, (M−H)⁻=356.20

m.p. 182.3° C.

### Example 27

### 1-[2-(2,5-difluoro-4-hydroxyphenyl)-2-hydroxyethyl]-4-(3-fluorophenyl)piperidin-4-ol

27-A: 1-(2,5-difluoro-4-hydroxyphenyl)ethanone

To a stirred suspension of aluminium trichloride (43.7 g) in carbon disulfide (100 mL) was added chloroacetyl chloride (16.1 g) at room temperature, and the mixture was stirred for 1 hour. A solution of 2,5-difluorophenol (21.3 g) in carbon disulfide (50 mL) was added to the mixture. Whole was stirred under reflux for 16 hours and cooled to room temperature. The resulting mixture was poured onto ice-water and extracted with ethyl acetate. The combined organic layer was washed with water and brine, dried (sodium sulfate) and evaporated. The residue was purified by chromatography on silica gel, eluting with ethyl acetate/n-hexane (1:4 v/v), to afford the titled compound as a solid (21.5 g).

¹H NMR (300 MHz, CDCl₃) δ=8.67 (dd, J=11, 7 Hz, 1H), 6.78 (dd, J=11, 7 Hz, 1H), 6.21 (br, 1H), 2.60 (d, J=5 Hz, 3H) ppm.

27-B: 1-{2,5-difluoro-4-[(triisopropylsilyl)oxy]phenyl}ethanone

The title compound was prepared according to the procedure described in Example 2 from 1-(2,5-difluoro-4-hydroxyphenyl)ethanone (8.1 g) as a colorless oil.

¹H NMR (300 MHz, CDCl₃) δ=7.63 (dd, J=11, 7 Hz, 1H), 6.68 (dd, J=12,7 Hz, 1H), 2.59 (d, J=5 Hz, 3H), 1.85-1.25 (m, 3H), 1.11 (d, J=7 Hz, 18H) ppm.

27-C: 2-bromo-1-{2,5-difluoro-4-[(triisopropylsilyl)oxy]phenyl}ethanone

The title compound was prepared according to the procedure described in Example 4 from 1-{2,5-difluoro-4-[(triisopropylsilyl)oxy]phenyl}ethanone (12 g) as oil.

¹H NMR (300 MHz, CDCl₃) δ=7.69 (dd, J=11, 7 Hz, 1H), 6.70 (dd, J=12, 7 Hz, 1H), 4.47 (d, J=3 Hz, 2H), 1.36-1.20 (m, 3H), 1.11 (d, J=7 Hz, 18H) ppm.

27-D: 1-{2-(2.5-difluoro-4-[(triisopropylsilyl)oxy]phenyl}-2-hydroxyethyl)-4-(3-fluorophenyl)piperidin-4-ol

The title compound was prepared according to the procedure described in Example from 2-bromo-1-{2,5-difluoro-4-[(triisopropylsilyl)oxy]phenyl}ethanone (0.8 g) and 4-(3-fluorophenyl)piperidin-4-ol as a solid. The crude product was used in the next step without further purification.

27-E: 1-[2-(2,5-difluoro-4-hydroxyphenyl)-2-hydroxyethyl]-4-(3-fluorophenyl)piperidin-4-ol

The title compound was prepared according to the procedure described in Example from 1-(2-{2,5-difluoro-4-[(triisopropylsilyl)oxy]phenyl}-2-hydroxyethyl)-4-(3-fluorophenyl)piperidin-4-ol (0.8 g) as a solid (0.4 g).

¹H NMR (300 MHz, CDCl₃) δ=7.38-7.21 (m, 4H), 6.97 (t, J=7 Hz, 1H), 6.69 (dd, J=11, 7 Hz, 1H), 5.02 (d, J=10 Hz, 1H), 3.06 (d, J=12 Hz, 1H), 2.90-2.40 (m, 5H), 2.15 (dq, J=5, 13 Hz, 2H), 1.82-1.75 (m, 2H) ppm.

27-F: 1-[2-(2,5-difluoro-4-hydroxyphenyl)-2-hydroxyethyl]-4-(3-fluorophenyl)piperidin-4-ol hydrochloride

Hydrogen chloride (0.27 ml, 1.0 eq), 4.0 M solution in ethyl acetate, was added to a solution of 1-[2-(2,5-difluoro-4-hydroxyphenyl)-2-hydroxyethyl]-4-(3-fluorophenyl)piperidin-4-ol (0.4 g) in methyl alcohol (5 mL). The mixture was stirred for 1 hours at room temperature and concentrated in vacuo. The residue was crystallized from 2-propanol-diisopropylether to afford the titled compound as a white solid. (0.2 g).

¹H NMR (300 MHz, DMSO-d₆) δ=10.53 (br, 1H), 10.06 (br, 1H), 7.47-7.24 (m, 4H), 7.10 (t, J=9 Hz, 1H), 6.83 (dd, J=11, 7 Hz, 1H), 6.29 (br, 1), ppm *other proton signals were couldn't read for the broadening of peak.

MS (ESI); (M+H)⁺=368.01, (M−H)⁻=365.97

m.p. 207.0° C.

### Example 28

### 1-[2-(2,5-difluoro-4-hydroxyphenyl)-2-hydroxyethyl]-4-(6-methoxypyridin-3-yl)piperidin-4-ol

28-A: 1-(2-{2,5-difluoro-4-[(triisopropylsilyl)oxy]phenyl}-2-hydroxyethyl)-4-(6-methoxypyridin-3-yl)piperidin-4-ol

The title compound was prepared according to the procedure described in Example from 2-bromo-1-{2,5-difluoro-4-[(triisopropylsilyl)oxy]phenyl}ethanone (0.8 g) as a solid (0.38 g). The crude product was used in the next step without further purification.

28-B: 1-[2-(2,5-difluoro-4-hydroxyphenyl)-2-hydroxyethyl]-4-(6-methoxypyridin-3-yl)piperidin-4-ol hydrochloride

The title compound is prepared from 1-(2-{2,5-difluoro-4-[(triisopropylsilyl)oxy]phenyl}-2-hydroxyethyl)-4-(6-methoxypyridin-3-yl)piperidin-4-ol (0.38 g) inseared of -(2-{2,5-difluoro-4-[(triisopropylsilyl)oxy]phenyl}-2-hydroxyethyl)-4-(3-fluorophenyl)piperidin-4-ol according to the method described in Example 27 as a solid (0.16 g).

¹H NMR (300 MHz, DMSO-d₆) δ=10.58 (br, 1H), 10.01 (br, 1H), 8.25 (d, J=2 Hz, 1H), 7.74 (dd, J=9, 3 Hz, 1H), 7.26 (dd, J=7, 12 Hz, 1H), 6.9-6.7 (m, 2H), 5.29 (d, J=8 Hz, 1H), 3.85 (s, 3H), 3.6-3.5 (m, 2H), 3.43-3.20 (m, 4H), 2.5-2.3 (2H), 1.84 (t, J=14 Hz, 2H) ppm

MS (ESI); (M+)⁺=381.02, (M−H)⁻=378.96

m.p. 199.5° C.

### Example 29

### 1-[2-(2-chloro-4-hydroxyphenyl)-2-hydroxyethyl]-4-(6-methoxypyridin-3-yl)piperidin-4-ol

29-A: 1-{2-chloro-4-[(triisopropylsilyl)oxy]phenyl}ethanone

The title compound was prepared according to the procedure described in Example 2 from 1-(2,-chloro-4-hydroxyphenyl)ethanone (10.8 g) as a colorless oil.

¹H NMR (300 MHz, CDCl₃) δ=7.61 (d, J=9 Hz, 1H), 6.92 (d, J=2 Hz, 1H), 6.79 (dd, J=9, 2 Hz, 1H), 2.63 (s, 3H), 1.32-1.20 (m, 3H), 1.10 (d, J=7 Hz, 18H) ppm.

29-B: 2-bromo-1-{2-chloro-4-[(triisopropylsilyl)oxy]phenyl}ethanone

The title compound was prepared according to the procedure described in Example 4 from 1-{2-chloro-4-[(triisopropylsilyl)oxy]phenyl}ethanone (13 g) as oil.

¹H NMR (300 MHz, CDCl₃) δ=7.63 (d, J=9 Hz, 1H), 6.94 (d, J=2 Hz, 1H), 6.83 (dd, J=9, 2 Hz, 1H), 4.55 (s, 2H), 1.35-1.20 (m, 3H), 1.10 (d, J=7 Hz, 18H) ppm.

29-C: 1-(2-{2-chloro-4-[(triisopropylsilyl)oxy]phenyl}-2-hydroxyethyl)-4-(6-methoxypyridin-3-yl)piperidin-4-ol

The title compound was prepared according to the procedure described in Example from 2-bromo-1-{2-chloro-4-[(triisopropylsilyl)oxy]phenyl}ethanone (0.8 g) and 4-(6-methoxypyridin-3-yl)piperidin-4-ol (0.55 g) as a solid (0.33 g).

¹H NMR (300 MHz, CDCl₃) δ 8.30 (d, J=2 Hz, 1H), 7.72 (dd, J=9, 2 Hz, 1H), 7.47 (d, J=8 Hz, 1H), 6.86 (d, J=2 Hz, 1H), 6.82 (dd, J=8, 2 Hz, 1H), 6.75 (d, J=9 Hz, 1H), 5.12 (dd, J=10, 3 Hz, 1H), 3.93 (s, 3H), 3.76-3.72 (m, 2H), 3.09 (brd, J=11 Hz, 1H), 2.86-2.53 (m, 3H), 2.35 (dd, J=10, 13 Hz, 1H), 2.19-2.06 (m, 2H), 1.90-1.77 (m, 2H), 1.30-1.18 (m, 3H), 1.09 (d, J=7 Hz, 18H) ppm

29-D: 1-[2-(2-chloro-4-hydroxyphenyl)-2-hydroxyethyl]-4-(6-methoxypyridin-3-yl)piperidin-4-ol

The title compound was prepared according to the procedure described in Example from 1-(2-{2-chloro-4-[(triisopropylsilyl)oxy]phenyl}-2-hydroxyethyl)-4-(6-methoxypyridin-3-yl)piperidin-4-ol (0.32 g) as a solid (0.16 g).

¹H NMR (300 MHz, DMSO-d₆) δ 9.73 (br, 1H), 8.24 (d, J=3 Hz, 1H), 7.77 (dd, J=9, 3 Hz, 1H), 7.39 (d, J=9 Hz, 1H), 6.77-6.73 (m, 3H), 5.01-4.97 (br, 2H), 4.86 (s, 1H), 3.83 (s, 3H), 2.8-2.47 (4H), 2.40 (brd, J=6 Hz, 2H), 1.98-1.88 (m, 2H), 1.63-1.58 (m, 2H) ppm.

MS (ESI); (M+H)⁺=378.94, (M−H)⁻=376.89

m.p. 190.5° C.

1. A compound of the formula (I): 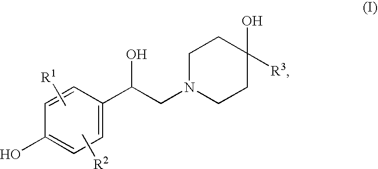
wherein R¹ and R² independently represents a hydrogen atom, a halogen atom or an alkyl group having from 1 to 6 carbon atoms;
R³ represents an aryl group having from 6 to 10 ring carbon atoms or a heteroaryl group having from 5 to 10 ring atoms which consists of from 1 to 4 heteroatoms independently selected from the group consisting of sulfur atoms, oxygen atoms and nitrogen atoms;
said aryl groups having from 6 to 10 ring carbon atoms and said heteroaryl groups having from 5 to 10 atoms are unsubstituted or are substituted by at least one substituent selected from the group consisting of substituents α;
said substituents α are selected from the group consisting of halogen atoms, alkyl groups having from 1 to 6 carbon atoms, alkoxy groups having from 1 to 6 carbon atoms or alkoxyalkyl groups having from 1 to 6 carbon atoms;
or a pharmaceutically acceptable ester of such compound,
or a pharmaceutically acceptable salt thereof.

2. A compound according to claim 1, wherein
R¹ and R² independently represents a hydrogen atom, a fluorine atom, a chlorine atom, or an alkyl group having from 1 to 4 carbon atoms.

3. A compound according to claim 1, wherein:
R³ represents an aryl group having from 6 to 7 ring carbon atoms or a heteroaryl group having from 5 to 10 ring atoms which consists of from 1 to 2 heteroatoms independently selected from the group consisting of sulfur atoms, oxygen atoms and nitrogen atoms.

4. A compound according to claim 1, wherein:
R³ represents a phenyl group, a thiazolyl group, an isothiazolyl group, an oxazolyl group, an isoxazolyl group, a pyrrolyl group, a pyridyl group, a pyrimidine group, a quinolyl group, an isoquinollyl group, a tetrahydroquinolyl group, a tetrahydroisoquinolyl group, a chromanyl group or an isochromanyl group.

5. A compound according to claim 1, wherein:
R³ represents a phenyl group, a thiazolyl group, a pyridyl group, or an isochromanyl group.

6. A compound according to claim 1 selected from
1-[2-(3-Fluoro-4-hydroxyphenyl)-2-hydroxyethyl]-4-(6-methoxypyridin-3-yl)-piperidin-4-ol methanesulfonate;
4-(3,4-Dihydro-1H-isochromen-7-yl)-1-[2-(3-fluoro-4-hydroxyphenyl)-2-hydroxyethyl]piperidin-4-ol methanesulfonate;
1-[2-(3-Fluoro-4-hydroxyphenyl)-2-hydroxyethyl]-4-(3-fluorophenyl)piperidin-4-ol methanesulfonate;
4-(3,4-Dihydro-1H-isochromen-7-yl)-1-[2-hydroxy-2-(4-hydroxy-3-methylphenyl)ethyl]piperidin-4-ol;
4-(3-Fluorophenyl)-1-[2-hydroxy-2-(4-hydroxy-3-methylphenyl)ethyl]piperidin-4-ol;
1-[2-Hydroxy-2-(4-hydroxy-3-methylphenyl)ethyl]-4-(6-methoxypyridin-3-yl)-piperidin-4-ol;
1-[2-(2-Fluoro-4-hydroxyphenyl)-2-hydroxyethyl]-4-(3-fluorophenyl)piperidin-4-ol;
4-(3,4-Dihydro-1H-isochromen-7-yl)-1-[2-(2-fluoro-4-hydroxyphenyl)-2-hydroxyethyl]piperidin-4-ol;
1-[2-(2-Fluoro-4-hydroxyphenyl)-2-hydroxyethyl]-4-(6-methoxypyridin-3-yl)piperidin-4-ol;
4-(3-Fluorophenyl)-1-[2-hydroxy-2-(4-hydroxyphenyl)ethyl]piperidin-4-ol;
1-[2-hydroxy-2-(4-hydroxyphenyl)ethyl]-4-(6-methoxypyridin-3-yl)piperidin-4-ol;
1-[2-Hydroxy-2-(4-hydroxyphenyl)ethyl]-4-[4-(methoxymethyl)phenyl]piperidin-4-ol;
1-[2-Hydroxy-2-(4-hydroxy-3-methylphenyl)ethyl]-4-[4-(methoxymethyl)phenyl]piperidin-4-ol;
1-[2-Hydroxy-2-(4-hydroxy-3-methylphenyl)ethyl]-4-(5-methyl-1,3-thiazol-2-yl)piperidin-4-ol;
1-[2-Hydroxy-2-(4-hydroxy-3-methylphenyl)ethyl]-4-(3-methoxyphenyl)-piperidin-4-ol hydrochloride;
4-(6-Ethoxypyridin-3-yl)-1-[2-hydroxy-2-(4-hydroxy-3-methylphenyl)ethyl]-piperidin-4-ol;
1-[2-(2-Fluoro-4-hydroxy-5-methylphenyl)-2-hydroxyethyl]-4-(6-methoxypyridin-3-yl)piperidin-4-ol;
4-(6-Fluoro-5-methoxypyridin-2-yl)-1-[2-hydroxy-2-(4-hydroxy-3-methylphenyl)ethyl]piperidin-4-ol;
1-[2-(3-chloro-4-hydroxyphenyl)-2-hydroxyethyl]-4-(6-methoxypyridin-3-yl)piperidin-4-ol hydrochloride;
1-2-(3-chloro-4-hydroxyphenyl)-2-hydroxyethyl]-4-[4-(methoxymethyl)phenyl]piperidin-4-ol;
1-[2-(2, 5-difluoro-4-hydroxyphenyl)-2-hydroxyethyl]-4-(3-fluorophenyl)piperidin-4-ol; and
1-[2-(2, 5-difluoro-4-hydroxyphenyl)-2-hydroxyethyl]-4-(6-methoxypyridin-3-yl)piperidin-4-ol; or a pharmaceutically acceptable salt thereof.

7. A compound according to claim 1 selected from
1-[2-(3-Fluoro-4-hydroxyphenyl)-2-hydroxyethyl]-4-(3-fluoropheny)piperidin-4-ol methanesulfonate;
4-(3,4-Dihydro-1H-isochromen-7-yl)-1-[2-hydroxy-2-(4-hydroxy-3-methylphenyl)ethyl]piperidin-4-ol;
4-(3-Fluorophenyl)-1-[2-hydroxy-2-(4-hydroxy-3-methylphenyl)ethyl]piperidin-4-ol;
1-[2-Hydroxy-2-(4-hydroxy-3-methylphenyl)ethyl]-4-(6-methoxypyridin-3-yl)-piperidin-4-ol;
1-[2-(2-Fluoro-4-hydroxyphenyl)-2-hydroxyethyl]-4-(3-fluorophenyl)piperidin-4-ol;
4-(3,4-Dihydro-1H-isochromen-7-yl)-1-[2-(2-fluoro-4-hydroxyphenyl)-2-hydroxyethyl]piperidin-4-ol;
4-(3-Fluorophenyl)-1-[2-hydroxy-2-(4-hydroxyphenyl)ethyl]piperidin-4-ol;
1-[2-hydroxy-2-(4-hydroxyphenyl)ethyl]-4-(6-methoxypyridin-3-yl)piperidin-4-ol;
1-[2-Hydroxy-2-(4-hydroxyphenyl)ethyl]-4-[4-(methoxymethyl)phenyl]piperidin-4-ol;
1-[2-Hydroxy-2-(4-hydroxy-3-methylphenyl)ethyl]-4-(3-methoxyphenyl)-piperidin-4-ol hydrochloride;
4-(6-Ethoxypyridin-3-yl)-1-[2-hydroxy-2-(4-hydroxy-3-methylphenyl)ethyl]-piperidin-4-ol;
1-[2-(2-Fluoro-4-hydroxy-5-methylphenyl)-2-hydroxyethyl]-4-(6-methoxypyridin-3-yl)piperidin-4-ol; and
4-(6-Fluoro-5-methoxypyridin-2-yl)-1-[2-hydroxy-2-(4-hydroxy-3-methylphenyl)ethyl]piperidin-4-ol;
or a pharmaceutically acceptable salt thereof.

8. A pharmaceutical composition, which comprises a compound according to claim 1, pharmaceutically acceptable ester of such compound, or a pharmaceutically acceptable salt thereof, and a pharmaceutically suitable acceptable carrier.

9. A pharmaceutical composition for the treatment of disease conditions caused by over activation of NMDA NR2B receptor, in a mammalian subject, which comprises a therapeutically effective amount of a compound according to claim 1, or a pharmaceutically acceptable ester of such compound, or a pharmaceutically acceptable salt thereof, and a suitable pharmaceutically acceptable carrier.

10. A pharmaceutical composition according to claim 9 where the disease condition is selected from stroke or brain injury, chronic neurodegenerative disease such as Parkinson's disease, Alzheimer's disease, Huntington's disease or amyotrophic lateral sclerosis (ALS), epilepsy, convulsive disorder, pain, anxiety, human immunodeficiency virus (HIV) related neuronal injury, migraine, depression, schizophrenia, tumor, post-anesthesia cognitive decline (PACD), glaucoma, tinnitus, tradive dyskinesia, allergic encephalomyelitis, opioid tolerance, drug abuse, alcohol abuse and Irritable bowel syndrome (IBS).

11. A method for the treatment of disease conditions caused by over activation of NMDA NR2B receptor, in a mammalian subject, which comprises administering to said subject a therapeutically effective amount of a compound according to claim 1, or a pharmaceutically acceptable ester of such compound, or a pharmaceutically acceptable salt thereof.

12. A method according to claim 11 where the disease condition is selected from stroke or brain injury, chronic neurodegenerative disease such as Parkinson's disease, Alzheimer's disease, Huntington's disease or amyotrophic lateral sclerosis (ALS), epilepsy, convulsive disorder, pain, anxiety, human immunodeficiency virus (HIV) related neuronal injury, migraine, depression, schizophrenia, tumor, post-anesthesia cognitive decline (PACD), glaucoma, tinnitus, tradive dyskinesia, allergic encephalomyelitis, opioid tolerance, drug abuse and alcohol abuse.

13. (canceled)

14. (canceled)
